(19) United States
(12) Patent Application Publication
(10) Pub. No.: US 2026/0233214 A1
(43) Pub. Date: Aug. 13, 2026

(54) Cationic Polymers And Methods Of Making Same, And Uses Thereof

(71) Applicants: Cornell University, Ithaca, NY (US); Carnegie Mellon University, Pittsburgh, PA (US)

(72) Inventors: Brett P. Fors, Ithaca, NY (US); Geoffrey W. Coates, Lansing, NY (US); Jesse H. Hsu, Ithaca, NY (US); Cheyenne Peltier, Ithaca, NY (US); Cassandra Ann Haynes, Sayre, PA (US); Kevin J.T. Noonan, Pittsburgh, PA (US)

(21) Appl. No.: 19/157,431

(22) PCT Filed: Feb. 20, 2024
(86) PCT No.: PCT/US2024/016479

§ 371 Date: Aug. 18, 2025

Related U.S. Application Data
(60) Provisional application No. 63/485,716, filed on Feb. 17, 2023.

(51) Int. Cl. B01J 41/14; B01J 41/05; C08F 232/08; C08F 297/06

(52) U.S. Cl. CPC B01J 41/14; B01J 41/05; C08F 232/08; C08F 297/06

(57) ABSTRACT

Cationic compounds and methods of making and using same. A cationic compound comprises one or more norbornenyl group(s) and one or more cationic group(s). The cationic groups(s) is/are chosen from phosphonium groups, imidazolium groups, cyclic ammonium groups, and the like, and any combination thereof. The cationic group(s) is/are independently covalently linked or covalently linked via a linking group to the norbornene ring of a norbornenyl group. In various examples, one or more cationic compound(s) is/are used as a monomer or monomers in a polymerization, such as, for example, a direct insertion polymerization or the like, to form a cationic polymer. In various examples, an anionic exchange membrane, which may be used in a device, such as, for example, in a sensor, an actuator, an energy-storage device, or an energy-generating device, or the like, comprising one or more cationic polymer(s).

### CROSS REFERENCE TO RELATED APPLICATIONS

This application claims the benefit of U.S. Provisional Application No. 63/485,716, filed Feb. 17, 2023; the contents of the above-identified application are hereby fully incorporated herein by reference in their entirety.

### STATEMENT REGARDING FEDERALLY SPONSORED RESEARCH

This invention was made with government support under contract no. DE-SC-0019445 awarded by the Department of Energy. The government has certain rights in the invention.

### BACKGROUND OF THE DISCLOSURE

Polymers with covalently incorporated ions have been known to give tough and stretchable materials with increased recyclability. Furthermore, their self-healing, energy dispersive, shape-memory, stimuli-responsive, biocompatible, and antimicrobial properties have made them particularly attractive for use as biomimetic actuators and sensors, and as gene and drug delivery systems. Importantly, ionic polymers and polyelectrolytes have especially proven useful as ion transport membranes for electrodialysis, redox flow batteries, water electrolysis, and fuel cell devices.

Anion Exchange Membranes (AEMs) are state-of-the-art solid electrolyte materials composed of cationic groups immobilized on a polymer backbone. These electronically insulating materials are known to transport hydroxide, making them key components in electrochemical devices such as anion exchange membrane water electrolyzers (AEMWEs) and fuel cells (AEMFCs), which are used to generate renewable, carbon-free hydrogen fuel and efficiently convert this fuel to electricity. In contrast to devices which operate under acidic conditions (e.g., proton exchange membrane fuel cells (PEMFCs)), AEMWEs and AEMFCs benefit from better catalyst stability and more facile oxygen electroreduction kinetics under alkaline conditions, enabling the use of non-platinum-group metal electrocatalysts to provide devices with significantly reduced costs. Importantly, AEMs are often the limiting component in these devices, and because the green hydrogen economy is anticipated to play an important role in reducing society's dependence on fossil fuel and combustion-based technologies, AEMs have received significant research interest in the last decade.

AEM design typically focuses on two criteria: hydroxide ion conductivity and polymer stability in alkaline media. Although several studies have reported AEMs with high hydroxide ion conductivity (above 80-100 mS/cm at 80° C.), the synthesis of AEMs with long-term alkaline stability remains a challenge. In this regard, aliphatic polymer backbones are uniquely suited for AEMs, as they lack the alkaline instability of heteroatoms, or the oxidative degradation and electrocatalyst adsorption associated with aromatic groups. In addition, aliphatic polymers avoid the use of per- and polyfluoroalkyl substances (PFAS), which are toxic and environmentally persistent byproducts of fluorinated polymers such as Nafion, an ion exchange membrane currently used in commercial PEMFCs.

A variety of aliphatic polymer-based AEMs through ring-opening metathesis polymerization (ROMP) have been accessed, generating materials with exceptional conductivity and stability; however, this route requires post-polymerization hydrogenation which can complicate rapid diversification and production of these materials. In 2019, Kohl and co-workers reported a vinyl addition polynorbornene (PNB) material which was modified to have pendent trimethylammonium cations. This robust PNB membrane exhibited remarkable hydroxide ion conductivity and was later crosslinked to achieve even higher performances; however, this vinyl addition material also required a post-polymerization modification route. Alternatively, Mecking and co-workers reported a direct coordination-insertion polymerization for imidazolium-functionalized polyethylene, but this strategy was limited to a cation incorporation of 0.67 mol %, despite a significantly increased loading of imidazolium monomer. Thus, a direct synthesis of aliphatic polymers with high ion incorporations remains a challenge.

In addition to the polymer backbone, the identity of the cation has proven important in AEM design. In a model compound study, only tetrakis(dialkylamino)phosphonium and aryl-substituted imidazolium cations exhibited zero degradation after 30 days in 2 M KOH/CD₃OH at 80° C. These ultra-stable cations have been incorporated into AEMs; however, examples remain limited as post-polymerization functionalization reactions of these more sterically demanding cations can require days to reach completion, and existing methods to directly polymerize these groups into AEMs either require post-polymerization hydrogenation or do not result in alkaline-stable membranes with robust physical properties. In 2017, Jannasch and co-workers systematically studied the effect of different tetraalkylammonium cations on the performance of poly(phenylene oxide) AEMs: however, due to the synthetic complications discussed above, only one such study has been conducted with alkaline-stable, aliphatic polymer-based AEMs, and none have been conducted with cation classes beyond tetraalkylammoniums. Thus, a one-step direct polymerization strategy to generate a diverse library of aliphatic polymer-based AEMs would provide a significant advancement.

The limited number of methods to directly polymerize ionic monomers currently hinders rapid diversification and production of ionic polymeric materials, namely anion exchange membranes (AEMs) which are important components in emerging alkaline fuel cell and electrolyzer technologies.

### SUMMARY OF THE DISCLOSURE

In an aspect, the present disclosure provides cationic compounds. In various examples, a cationic compound comprises one or more cationic group(s). In various examples, a cationic compound is suitable for use (or is used) as monomer (e.g., in a polymerization, such as, for example, a polymerization of the present disclosure or the like). In various examples, a cationic compound is suitable for use as a monomer in direct insertion polymerization or the like. In various examples, a cationic compound comprises one or more norbornenyl group(s) and at least one cationic group, where each cationic group is covalently linked (e.g., directly linked (such as, for example, fused to) or linked via a linking group) to the norbornene ring of a norbornenyl group. In various examples, the cationic group(s) is/are independently at each occurrence chosen from phosphonium groups (such as, for example, acyclic phosphonium groups and cyclic phosphonium groups), imidazolium groups, cyclic ammonium groups (e.g., where the cyclic ammonium groups are directly covalently linked or covalently linked via a linking group to the norbornene ring of a norbornenyl group), and the like. In various examples, the cationic compound (e.g., a monomer) is an endo isomer or an exo isomer (e.g., a cationic group is a substituent in an endo or exo position of a norbornenyl group. In various examples, the cationic compound is a salt, a partial salt, a solvate, a polymorph, or the like, or a stereoisomer or a mixture of stereoisomers, an isotopic variant, a tautomer, or the like.

In an aspect, the present disclosure provides cationic polymers. In various examples, a cationic polymer comprises at least a portion of the or one or more or all repeat units formed from or derived from a cationic compound (which is a cationic monomer) of the present disclosure. A cationic polymer may be referred to, in the alternative, as an ionomer or ionic polymer. In various examples, a cationic polymer is made by a method of the present disclosure.

In various examples, a cationic polymer comprises repeat units comprising one or more norbornyl group(s), where at least a portion of the or at least one or more or all norbornyl group(s) comprise(s) at least one cationic group. In various examples, the polymer is a homopolymer or a copolymer.

In an aspect, the present disclosure provides methods of making cationic polymers. In various examples, a method results in formation of a cationic polymer or cationic polymer(s) of the present disclosure. In various examples, a method comprises a direct insertion polymerization or the like.

In various examples, a method of making a cationic polymer comprises: polymerizing one or more cationic monomer(s) independently chosen from cationic monomer(s) of the present disclosure, and optionally, one or more crosslinker(s) (such as, for example, cationic crosslinker(s), one or more charge neutral crosslinking monomer(s), non-ionic crosslinking monomer(s), or the like, or any combination thereof); or copolymerizing one or more monomer(s) independently chosen from cationic monomer(s) of the present disclosure and one or more co-monomer(s) that do not comprise a cationic group (such as, for example, monomers other than cationic monomer(s) of the present disclosure), and optionally, one or more crosslinker(s) (such as, for example, cationic crosslinker(s), one or more charge neutral crosslinking monomer(s), non-ionic crosslinking monomer(s), or the like, or any combination thereof), where the polymerization is a direct insertion polymerization.

In an aspect, the present disclosure provides anion exchange membranes. In various examples, an anion exchange membrane comprises one or more cationic polymer(s) of the present disclosure. An anion exchange membrane can be used in various devices.

In an aspect, the present disclosure provides devices. In various examples, a device comprises one or more cationic polymer(s) of the present disclosure. In various examples, a device is a sensor, an actuator an energy-storage device, an energy-generating device, or the like, or any combination thereof. In various example, an actuator or sensor is a biomimetic actuator or sensor or the like. In various examples, an electrochemical device is a battery, a fuel cell, a water-electrolysis device, a water electrolyzer, an electrodialysis device, or the like.

In an aspect, the present disclosure provides uses of cationic compounds and polymers of the present disclosure. In various examples, one or more cationic compound(s) is/are used as monomer(s) in a polymerization reaction. In various examples, one or more cationic compound(s) is/are used as monomer(s) in a polymerization reaction of the present disclosure or the like. In various examples, one or more cationic compound(s) is/are used as materials for gene/drug delivery or the like. In various examples, one or more cationic compound(s) are used as (or is/are) anti-fouling agents and/or antimicrobial agents, or the like.

### BRIEF DESCRIPTION OF THE FIGURES

For a fuller understanding of the nature and objects of the disclosure, reference should be made to the following detailed description taken in conjunction with the accompanying figures.

FIG. 1 shows direct coordination-insertion polymerization of ionic monomers to rapidly produce a diverse library of materials.

FIG. 2 shows a reaction scheme surveying the effect of different anions on the activity of the polymerization catalyst (above), and monomer conversions after 30 minutes in the absence or presence of different tetrabutylammonium salts (below), with error bars representing 95% confidence intervals.

FIG. 3 shows a) overall scheme for the direct insertion polymerization of ionic monomers, and corresponding conversion vs. time plots for polymerizations of monomers with b) tetrabutylammonium, c) piperidinium, d) phosphonium, e) imidazolium, and f) trimethylammonium cations.

FIG. 4 shows sequential addition of monomers to access a block copolymer structure via direct insertion polymerization.

FIG. 5 shows a) images of clear polymer films placed in front of their respective chemical structures printed on white paper, b) plot of hydroxide conductivity at 80° C. vs. water uptake for all polymers, c) graph of hydroxide conductivity at 80° C. divided by IEC^{theo} as a function of the cation in each membrane, and d) plot of hydroxide conductivity at 22° C. vs. time in 1 M KOH at 80° C. for all membranes, with error bars representing 95% confidence intervals.

FIG. 6 shows a) Piperidinium membrane composition (left) and membrane electrode assembly (MEA) after operation (right), b) AEM fuel cell performance using the piperidinium membrane shown above.

FIG. 7 shows representative GPC traces for polymerizations of hexNB in the presence of different tetrabutylammonium salts.

FIG. 8 shows Conversion vs. time plot for the polymerization of hexNB.

FIG. 9 shows Semilogarithmic plot for the polymerization of hexNB.

FIG. 10 shows Stacked ¹H NMR spectra of aliquots from the copolymerization of tbaNB and hexNB over 25 h. Peak heights were normalized to the internal standard (anisole).

FIG. 11 shows Conversion vs. time plot for the copolymerization of tbaNB and hexNB.

FIG. 12 shows Semilogarithmic plot for the copolymerization of tbaNB and hexNB, where each trendline includes timepoints from before the exo monomers were completely consumed.

FIG. 13 shows Conversion vs. time plot for the copolymerization of pipNB and hexNB.

FIG. 14 shows Semilogarithmic plot for the copolymerization of pipNB and hexNB.

FIG. 15 shows Conversion vs. time plot for the copolymerization of phosNB and hexNB.

FIG. 16 shows Semilogarithmic plot for the copolymerization of phosNB and hexNB, where each trendline includes timepoints from before the exo monomers were completely consumed.

FIG. 17 shows Conversion vs. time plot for the copolymerization of imidNB and hexNB.

FIG. 18 shows Semilogarithmic plot for the copolymerization of imidNB and hexNB, where each trendline includes timepoints from before the exo monomers were completely consumed.

FIG. 19 shows Conversion vs. time plot for the copolymerization of tmaNB and hexNB.

FIG. 20 shows Semilogarithmic plot for the copolymerization of tmaNB and hexNB.

FIG. 21 shows Percent cation in polymer vs. total monomer conversion plot for copolymerizations of all five cation-containing monomers, where the percent cation in polymer is defined as the percent of consumed monomer that is the cation-containing monomer.

FIG. 22 shows Percent exo in polymer vs. total monomer conversion plot for copolymerizations of all five cation-containing monomers, where the percent exo in polymer is defined as the percent of consumed monomer that is the exo isomer.

FIG. 23 shows GPC traces for all membranes, with values reported in Table 1 and FIG. 3.

FIG. 24 shows GPC traces for the polymerization of hexNB and subsequent chain extension to form a hexNB-b-tbaNB diblock copolymer.

FIG. 25 shows Hydroxide conductivity at 22° C. (σ^{22° C.}) vs. temperature plot for membranes with piperidinium, tetrabutylammonium, phosphonium, and imidazolium on day 1 after equilibration and trimethylammonium on day 3 after equilibration.

FIG. 26 shows Hydroxide conductivity at 22° C. (σ^{22° C.}) divided by the maximum hydroxide conductivity at 22° C. vs. time for all membranes.

FIG. 27 shows Water uptake (WU) vs. theoretical ion exchange capacity (IEC^{theo}) for all membranes with error calculated to 95% confidence.

FIG. 28 shows Hydroxide conductivity at 80° C. (σ^{80° C.}) vs. theoretical ion exchange capacity (IEC^{theo}) for all membranes.

FIG. 29 shows GPC trace of the piperidinium membrane used in the membrane electrode assembly.

FIG. 30 shows Image of the membrane electrode assembly after operation with the piperidinium membrane that was 25±6 μm thick, with error calculated to 95% confidence.

FIG. 31 shows Plot of high-frequency resistance (HFR, obtained at 5000 Hz under the conditions of the instant fuel cell test) vs. the current density range of the fuel cell test. The reported HFR (125 mΩ cm²) is the average HFR over the above current density range (0.0 V to 2.0 V).

FIG. 32 shows a scheme of the procedure for solvent casting polymer films.

FIG. 33 shows examples of cationic polymers and cationic compounds of the present disclosure.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise stated, “about,” “approximately,” “substantially,” or the like, when used in connection with a measurable variable such as, for example, a parameter, an amount, a temporal duration, or the like, are meant to encompass variations of, for example, a specified value including, for example, those within experimental error (which can be determined by for example, a given data set, an art accepted standard, and/or with a given confidence interval (e.g. 90%, 95%, or more confidence interval from the mean), such as, for example, variations of +/−10% or less, +/−5% or less, +/−1% or less, and +/−0.1% or less of and from the specified value) or to encompass alternatives to the members of the list that would be recognized by one of ordinary skill in the art as alternatives, where the members and the alternatives may define a genus or sub-genus, insofar such variations are appropriate to perform in the context of the disclosure. As used herein, unless otherwise stated, the terms “about,” “approximate,” “at or about,” and “substantially” can mean that the amount or value in question can be the exact value or a value that provides equivalent results or effects as recited in the sample claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that, for example, equivalent results, effects, or the like are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also, unless otherwise stated, include individual values (e.g., about 1%, about 2%, about 3%, and about 4%) and the sub-ranges (e.g., about 0.5% to about 1.1%; about 0.5% to about 2.4%; about 0.5% to about 3.2%, and about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

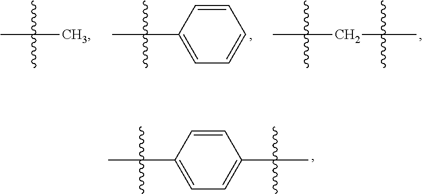

and the like.

As used herein, unless otherwise stated, “aliphatic group” refers to branched or unbranched hydrocarbons and includes alkanes, alkenes, alkynes, and the like. In various examples, an aliphatic group is a C₁ to C₂₀ aliphatic group. In various examples, an aliphatic group is unsubstituted or substituted with one or more substituent(s). Examples of substituents include, but are not limited to, various substituents such as, for example, halide groups (—F, —Cl, —Br, and —I), alkene groups, alkyne groups, aliphatic groups, aryl groups, alkoxide groups, carboxylate groups, carboxylic acid groups, ether groups, and the like.

As used herein, unless otherwise stated, the term “alkyl group” refers to branched or unbranched hydrocarbon groups that include only single bonds between carbon atoms (not including substituent(s), if any). In various examples, an alkyl group is a C₁ to C₁₀ alkyl group (e.g., C₁, C₂, C₃, C₄, C₅, C₆, C₇, C₈, C₉, or C₁₀), including all integer numbers of carbons and ranges of numbers of carbons therebetween. In various examples, an alkyl group is a saturated group. In various examples, an alkyl group is a cyclic alkyl group, e.g., a monocyclic alkyl group or a polycyclicalkyl group or the like. Examples of alkyl groups include, but are not limited to, methyl groups, ethyl groups, propyl groups, butyl groups, isopropyl groups, tert-butyl groups, cyclohexyl groups, adamantyl groups, and the like. In various examples, an alkyl group is unsubstituted or substituted with one or more substituent(s). Examples of substituents include, but are not limited to, various substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl group, amine groups, nitro group, cyano groups, isocyano groups, silane groups (e.g., alkyl silane groups, aryl silane groups, alkyl/aryl silane groups, or the like), alkoxide groups, 25 alcohol groups, ether groups, ketone groups, carboxylate groups, carboxylic acid groups, ester groups, amide groups, thioether groups, carbamate groups, carboxylic acid groups and the like, and any combination thereof.

As used herein, unless otherwise indicated, the term “aryl group” refers to C₅ to C₃₀ aromatic or partially aromatic carbocyclic groups. In various examples, an aryl group is a C₅ to C₃₀ aromatic or partially aromatic carbocyclic group (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀), including all integer numbers of carbons and ranges of numbers of carbons therebetween. In various examples, an aryl group comprises (or is) one or more polyaryl group(s) (such as, for example, fused ring group(s), biaryl group(s), or the like, or any combination thereof) or the like, or any combination thereof. In various examples, an aryl group is unsubstituted or substituted with one or more substituent(s). Examples of substituents include, but are not limited to, substituents such as, for example, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, hydroxyl groups, amine groups, nitro groups, cyano groups, isocyano groups, silyl groups, alkoxide groups, alcohol groups, ether groups, ketone groups, carboxylate groups, carboxylic acid groups, ester groups, amide groups, thioether groups, silane groups (e.g., alkyl silane groups, aryl silane groups, alkyl/aryl silane groups, or the like), and the like, and any combination thereof. In various examples, an aryl group comprises one or more heteroatom(s) (such as, for example, oxygen(s), nitrogen(s) (e.g., pyridinyl groups and the like), sulfur(s), and the like, and any combination thereof), which may be referred to, in the alternative, as a heteroaryl group. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, pyrrolyl groups, thiophenyl groups, benzofuranyl groups, indolyl groups, imidazolyl groups, benzimidazolyl groups, pyridinyl groups, and the like.

As used herein, unless otherwise stated, the term “structural analog” refers to any cationic compound (e.g., monomer or the like) or group that can be envisioned to arise from an original cationic compound (e.g., monomer or the like) or group if one atom or group of atoms, functional groups, or substructures is replaced with another atom or group of atoms, functional groups, substructures, or the like. In various examples, the term “structural analog” refers to any group that is derived from an original any cationic compound (e.g., monomer or the like) or group by a chemical reaction, where the cationic compound (e.g., monomer or the like) or group is modified or partially substituted such that at least one structural feature of the cationic compound (e.g., monomer or the like) or group is retained.

The present disclosure provides, inter alia, cationic compounds (which may be cationic monomers), cationic polymers, and methods of making cationic polymers. The present disclosure also provides uses of the cationic compounds and cationic polymers.

In an aspect, the present disclosure provides cationic compounds. In various examples, a cationic compound comprises one or more cationic group(s). In various examples, a cationic compound is suitable for use (or is used) as monomer (e.g., in a polymerization, such as, for example, a polymerization of the present disclosure or the like). In various examples, a cationic compound is suitable for use as a monomer in direct insertion polymerization or the like. Non-limiting examples of compositions are described herein.

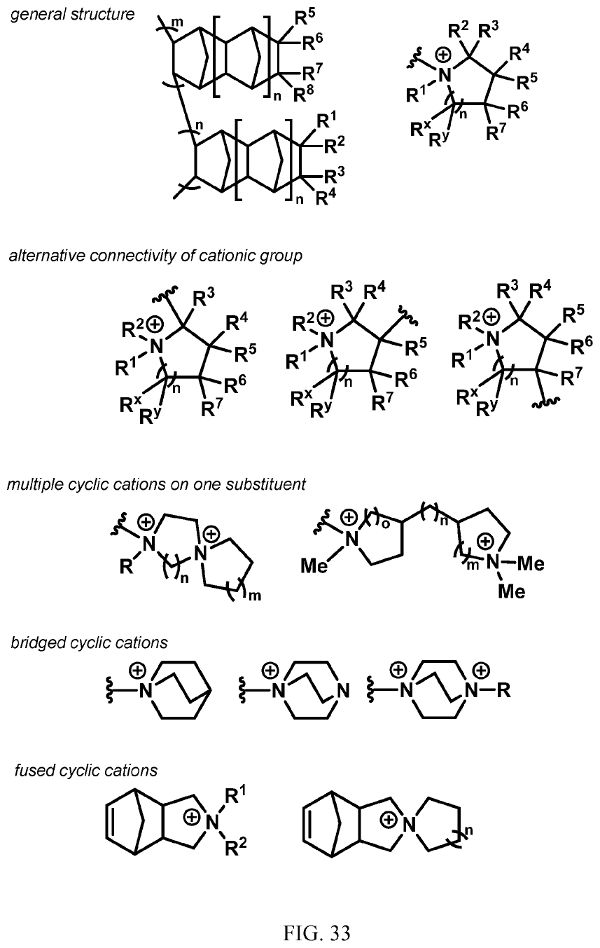

In various examples, a cationic compound comprises one or more norbornenyl group(s) and at least one cationic group, where each cationic group is covalently linked (e.g., directly linked (such as, for example, fused to) or linked via a linking group) to the norbornene ring of a norbornenyl group. In various examples, the cationic group(s) is/are independently at each occurrence chosen from phosphonium groups (such as, for example, acyclic phosphonium groups and cyclic phosphonium groups), imidazolium groups, cyclic ammonium groups (e.g., where the cyclic ammonium groups are directly covalently linked or covalently linked via a linking group to the norbornene ring of a norbornenyl group), and the like. In various examples, a cationic compound (e.g., a monomer) is an endo isomer or an exo isomer (e.g., a cationic group is a substituent in an endo or exo position of a norbornenyl group. In various examples, a cationic compound is a salt, a partial salt, a solvate, a polymorph, or the like, or a stereoisomer or a mixture of stereoisomers, an isotopic variant, a tautomer, or the like. In various examples, these cationic compounds are cationic monomers (which may be referred to, in the alternative, as ionic monomers. Non-limiting examples of cationic compounds are shown in FIG. 33.

In various examples, a cationic compound comprises the following structure:

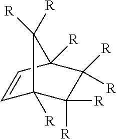

and structural analogs thereof, or a salt, a partial salt, a solvate, a polymorph, or the like thereof, or a stereoisomer or a mixture of stereoisomers, an isotopic variant, a tautomer, or the like thereof. In various examples, R is independently at each occurrence chosen from H group, alkyl group (e.g., C₁-C₂₀ alkyl group, including all integer number of carbons therebetween), cationic group (e.g., (e.g., cationic group covalently linked to the norbornene ring directly or via a linking group) chosen from phosphonium groups, imidazolium groups, cyclic ammonium groups and the like, and the like, with the proviso that at least one R group is a cationic group (e.g., cationic group covalently linked to the norbornene ring directly or via a linking group). In various examples, a cationic compound comprises the following structure:

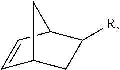

or a structural analog thereof, or the like. In various examples, a cationic compound (e.g., a cationic monomer) is an endo isomer or an exo isomer. In various examples, R is as defined above. In various examples, an R group is independently in the endo or exo position. As illustrative examples, a cationic compound comprises the following structure:

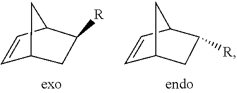

or the like.

In various examples, each cationic group is covalently linked directly or via a linking group to the norbornene ring of a norbornenyl group. In various examples, each cationic group is covalently linked to the norbornene ring (e.g., directly linked or linked via a linking group) via a carbon other than a carbon of the carbon-carbon double bond of the norbornenyl group. In various examples, each cationic group is covalently linked to the norbornene ring (e.g., directly linked or linked via a linking group) via a carbon at the endo or exo position other than a carbon of the carbon-carbon double bond of the norbornenyl group.

A cationic compound can comprise various cationic groups. In various examples, a cationic compound comprises phosphonium group(s) or imidazolium groups or cyclic ammonium groups, or the like, or any combination thereof. In various examples, a cationic compound comprises one or more phosphonium group(s). In various examples, a phosphonium group comprises the following structure:

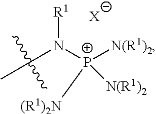

or a structural analog thereof. In various examples, R¹ is independently at each occurrence chosen from H group, alkyl groups (such as, for example, a C₁, C₂, C₃, C₄, C₅, and C₆ alkyl groups and the like), cyclic alkyl groups, aryl groups, and the like, and X⁻ is an anion (such as, for example, a halide anion (e.g., F⁻, Cl⁻, Br⁻— or I⁻) or a complex anion (such as, for example, BF₄⁻, SbF₆⁻, SbCl₆⁻, PF₆⁻, B(Ar)₄⁻, where Ar is an aryl group, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), and NTf₂⁻, where Tf is a triflate group, and the like), or the like). In various examples, an imidazolium group comprises the following structure:

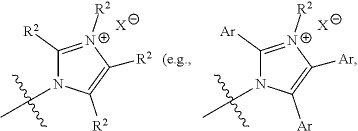

where Ar is independently at each occurrence an aryl group, and the like) or a structural analog thereof. In various examples, R² is independently at each occurrence chosen from H, alkyl groups (such as, for example, a C₁, C₂, C₃, C₄, C₅, and C₆ alkyl groups and the like), cyclic alkyl groups, aryl groups, and the like and X⁻ is an anion (such as, for example, a halide anion (e.g., F⁻, Cl⁻, Br⁻— or I⁻) or a complex anion (such as, for example, BF₄⁻, SbF₆⁻, SbCl₆⁻, PF₆⁻, B(Ar)₄⁻, where Ar is an aryl group, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), and NTf₂⁻, where Tf is a triflate group, and the like) or the like). In various examples, a cyclic ammonium group comprises the following structure:

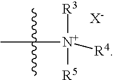

In various examples, (i) R³, R⁴, and the ammonium nitrogen (N⁻) taken together form a heterocyclic group where N⁺ is a member of the heterocyclic ring (e.g., a piperidinium group or the like) and R⁵ is an aliphatic or aryl group, or (ii) R³, R⁴, R⁵, and N taken together form an aliphatic group-bridged heterocyclic group where N⁺ is a member of the heterocyclic ring (e.g., a quinuclidinium group or the like), or the like. In various examples, the cyclic ammonium group(s) is/are covalently linked via a linking group to the norbornene ring of a norbornenyl group. In various examples, the cyclic ammonium group(s) is/are not directly covalently linked to the norbornene ring of a norbornenyl group. In various examples, a cyclic ammonium group is covalently linked directly or via a linking group to the norbornene ring of a norbornenyl group.

In various examples, a cationic compound comprises 2, 3, 4, or 5 cationic groups covalently linked (e.g., directly or via a linking group) to the norbornenyl group. These cationic compounds may be referred to, in the alternative, as multifunctional cationic compounds (or multifunctional cationic monomers).

In various examples, a cationic compound comprises at least two cationic groups covalently linked directly to each other via a linking group. In various examples, a cationic compound comprises at least two cationic groups, where one (e.g., primary or first) cationic group is covalently linked (e.g., directly or via a linking group, such as, for example, via a carbon (which may be an endo or exo position) other than a carbon of the carbon-carbon double bond of the norbornenyl group) to a norbornenyl group (e.g., a norbornenyl group suitable for direct insertion polymerization or the like) and one or more additional (e.g., secondary or second) cationic groups(s) covalently linked (e.g., directly or via a linking group) to the one (e.g., primary) cationic group. In various examples, at least one of the cationic groups is a phosphonium group, an imidazolium group, or the like. These cationic compounds may be referred to, in the alternative, as multifunctional cationic compounds. In various examples, these cationic compounds are multifunctional cationic monomers. Non-limiting examples of linking groups include aliphatic groups, aryl groups, heterocyclic groups, and the like. In various examples, a cationic compound comprises two cationic groups linked directly to each other via an aliphatic group, a heterocyclic group, where at least one of the N atoms is quaternary, or a bridged heterocyclic group where at least one of the N atoms is a quaternary atom of a first quaternary ammonium group, or the like and an aliphatic group (e.g., a second aliphatic group), a heterocyclic group (e.g., a second heterocyclic group) where at least one of the N atoms is quaternary, or a bridged heterocyclic group (e.g., a second bridged heterocyclic group) where at least one of the N atoms is a quaternary atom of a second quaternary group, or the like (any of which may be linking groups).

In various examples, a cationic compound comprises at least two norbornenyl groups covalently linked (e.g., directly or via a linking group) to a cationic group. In various examples, a cationic compound comprises 2, 3, 4, or 5 norbornenyl groups covalently linked (e.g., independently directly linked or via a linking group) to a cationic group.

In various examples, a cationic compound comprises a cationic group and at least two norbornenyl groups (e.g., a norbornenyl group suitable for direct insertion polymerization or the like), where each of the norbornenyl groups are covalently linked (e.g., directly or via a linking group, such as, for example, via a carbon (which may be an endo or exo position) other than a carbon of the carbon-carbon double bond of the norbornenyl group) to the cationic group. In various examples, these cationic compounds are cationic crosslinking monomers.

In various examples, a cationic compound comprises at least two norbornenyl groups or more covalently linked to a cationic group (e.g., independently directly linked or via a linking group). In various examples, a cationic compound comprises 2, 3, 4, or 5 norbornenyl groups covalently linked to a cationic group (e.g., independently directly linked or via a linking group).

In various examples, a cationic group independently at each occurrence is directly covalently linked to the norbornenyl group or is covalently linked to the norbornene ring via a linking group. In various examples, a cationic group is covalently linked to the norbornene ring via a linking group comprising the following structure: -L-CG, where L is a linking group (e.g., an alkyl group (such as, for example, a C₁-C₂₀ alkyl group, including all integer number of carbons therebetween), an aryl group, or the like) and CG is a cationic group).

In various examples, a cationic compound comprises a cyclic cationic group fused to a norbornenyl group (e.g., a cyclic cationic group is not covalently linked to a norbornenyl group via a linking group). As illustrative examples, a cationic compound comprises the following structure:

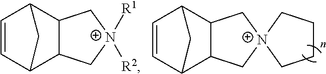

(e.g., where n is 1, 2, 3, or 4), or the like.

In various examples, a cationic compound further comprises one or more other cationic group(s). In various examples, the other cationic group(s) is/are independently chosen from quaternary ammonium groups (such as, for example, acyclic quaternary ammonium groups and the like) and the like. In various examples, a quaternary cationic group comprises the following structure:

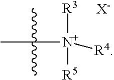

In various examples, R¹, R², and R³ are independently at each occurrence an aliphatic or aryl group (such as, for example, a C₁, C₂, C₃, C₄, C₅, and C₆ alkyl groups and the like). In various examples, an other cationic group independently at each occurrence is directly covalently linked to the norbornenyl group or is covalently linked to the norbornene ring via a linking group (e.g., comprising the following structure: -L-OCG, where L is a linking group (e.g., an alkyl group (such as, for example, a C₁-C₂₀ alkyl group, including all integer number of carbons therebetween), an aryl group, or the like) and OCG is an other cationic group).

In an aspect, the present disclosure provides cationic polymers. In various examples, a cationic polymer comprises at least a portion of the or one or more or all repeat units formed from or derived from a cationic compound (which is a cationic monomer) of the present disclosure. A cationic polymer may be referred to, in the alternative, as an ionomer or ionic polymer. In various examples, a cationic polymer is made by a method of the present disclosure. Non-limiting examples of cationic polymers are described herein.

In various examples, a cationic polymer comprises repeat units comprising one or more norbornyl group(s), where at least a portion of the or at least one or more or all norbornyl group(s) comprise(s) at least one cationic group. In various examples, a cationic polymer comprises repeat units independently formed from (e.g., via a) polymerization method of the present disclosure (such as, for example, a method of any one of Statements 10-15 or the like). In various examples, a polymer is a homopolymer or a copolymer. In various examples, a cationic polymer or one or more domain(s) of a cationic polymer is/are amorphous, or the like. In various examples, a cationic polymer does not comprise heteroatoms and/or aromatic groups and/or fluorine groups. In various examples, a cationic polymer does not comprise cationic group(s) formed by post-polymerization introduction of cationic group(s) (e.g., functionalization of a cationic polymer or the like). Non-limiting examples of cationic polymers are shown in FIG. 33.

In various examples, a cationic polymer, which may be a homopolymer or a copolymer, comprises the following structure:

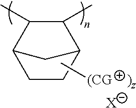

and the like and structural analogs thereof. In various examples, CG is cationic group (e.g., a cationic group directly covalently linked to the norbornyl group (such as, for example a non-backbone carbon) or linked via a linking group, a cationic group fused to the norbornyl group, or the like) and/or each cationic group of the cationic polymer is independently chosen from phosphonium groups, imidazolium groups, cyclic ammonium groups, and the like, and/or X⁻ is an anion (such as, for example, a halide anion (e.g., F⁻, Cl⁻, Br⁻— or I⁻) or a complex anion (such as, for example, BF₄⁻, SbF₆⁻, SbCl₆⁻, PF₆⁻, B(Ar)₄⁻, where Ar is an aryl group, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), and NTf₂⁻, where Tf is a triflate group, and the like) or the like), and/or z is independently at each occurrence 1, 2, 3, 4, or 5, or n is 1 to 10,000, including all integer n values and ranges therebetween (e.g., 2 to 10,000 or 200 to 1,000), or any combination thereof. In various examples, the mol fraction of the structure(s) (or this/these repeat unit(s)) in a cationic polymer is about 0.01 to about 1, including all 0.001 values and ranges therebetween.

In various examples, the cationic group(s) is/are independently at each occurrence directly covalently linked to the norbornyl group (e.g., a non-backbone carbon of the norbornyl group) or is covalently linked to the norbornyl group (e.g., a non-backbone carbon of the norbornyl group) via a linking group (e.g., comprising the following structure: -L-CG, where L is a linking group (e.g., an alkyl group (such as, for example, a C₁-C₂₀ alkyl group, including all integer number of carbons therebetween), an aryl group, or the like) and CG is a cationic group).

In various examples, a cationic polymer repeat unit comprises a cyclic cationic group fused to a norbornyl group (e.g., a cyclic cationic group is not covalently linked to a norbornyl group via a linking group). In various examples, at least a portion or all of the repeat units further comprise one or more other cationic group(s) (OCG(s)). In various examples, the other cationic group(s) is/are independently chosen from quaternary ammonium groups and the like. In various examples, a quaternary cationic group comprises the following structure:

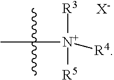

In various examples, R³, R⁴, and R⁵ are independently at each occurrence an aliphatic or aryl group (such as, for example, a C₁, C₂, C₃, C₄, C₅, and C₆ alkyl groups and the like).

In various examples, an other cationic group independently at each occurrence is directly covalently linked (e.g., a non-backbone carbon of the norbornyl group) to the norbornyl group or is covalently linked to the norbornyl group (e.g., a non-backbone carbon of the norbornyl group) via a linking group (e.g., comprising the following structure: -L-X where L is a linking group (e.g., an alkyl group (such as, for example, a C₁-C₂₀ alkyl group, including all integer number of carbons therebetween), an aryl group, or the like) and X is a cationic group (X⁺)).

In various examples, a cationic polymer is a copolymer. In various examples, a copolymer is a block copolymer, a random copolymer, a tapered copolymer, or the like. In various examples, a copolymer comprises two or more different repeat unit(s) (repeat units formed from a cationic compound (which is a cationic monomer) of the present disclosure). In various examples, a copolymer comprises one or more repeat unit(s) (repeat units formed from a cationic monomer of the present disclosure), which may be the same or different, and one or more additional repeat unit(s) (additional repeat units are repeat units other than those formed from a cationic monomer of the present disclosure), which may be the same or different.

A copolymer can comprise various additional repeat units. Additional repeat unit(s) may be present in a cationic polymer as one or more block(s), randomly distributed in the cationic polymer, or the like. In various examples, an additional repeat unit or additional repeat units is/are formed from monomer(s) that can be polymerized in direct insertion polymerizations and the like. In various examples, a cationic polymer is formed by copolymerization with norbornene, a structural analog thereof (which is not a cationic compound/cationic monomer of the present disclosure, such as, for example, a cationic compound of any one of claims 1-4 or the like), or the like, or any combination thereof. In various examples, an additional repeat unit or additional repeat units is/are chosen from hydrocarbon repeat units, and the like. In various examples, one or more or all of the repeat unit(s) comprises (or is) an aliphatic group, an aryl group (e.g., where at least a portion or all of which are heteroaryl groups), or the like.

In various examples, a cationic polymer is a copolymer and the copolymer further comprises one or more repeat unit(s) independently at each occurrence chosen from

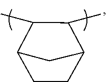

structural analogs thereof, and the like. In various examples, the repeat unit(s) is/are independently at each occurrence substituted with one or more substituent(s) independently chosen at each occurrence from alkyl groups (such as, for example, C₁ to C₁₀ alkyl groups or the like), aryl groups, or the like. In various examples, at least a portion of the or one or more or all cationic polymer repeat unit(s) further comprise(s) a cyclic group fused to a norbornyl group (e.g., a cyclic group is not covalently linked to a norbornyl group via a linking group).

In various examples, a cationic polymer is crosslinkable or crosslinked (e.g., intramolecularly crosslinked, intermolecularly crosslinked, or both). In various examples, a cationic polymer is a network polymer or the like. In various examples, a cationic polymer comprises a plurality of groups capable of forming (or is configured to form) crosslinking groups (e.g., intramolecular crosslinking groups, intermolecular crosslinking groups, or a combination thereof). In various examples, a cationic polymer comprises a plurality of crosslinking groups (e.g., a plurality of intramolecular crosslinking groups, a plurality of intermolecular crosslinking groups, or a combination thereof). In various examples, a cationic polymer is crosslinked as formed (e.g., as a result of a direct insertion polymerization or the like).

A cationic polymer can have various end groups. In various examples, the end group(s) is are independently chosen from end groups resulting from direct insertion polymerization reaction(s). In various examples, at least a portion or all of the end groups are chosen from H group, alkyl groups (e.g., a methyl group or the like), or the like, or any combination thereof.

An end group can result from post-direct insertion polymerization reaction(s) chosen to provide desired end groups. Suitable post polymerization reactions are known in the art. In various examples, at least a portion or all of the end groups are alkenyl groups or the like. In various examples, at least a portion of or all of the terminal carbons is/are, independently at each occurrence, substituted with an aryl group, hydrogen (H) group, an alkyl group, a halogen, a hydroxyl group, a palladium metal center (which may be a palladium metal center of a catalyst), and/or at least a portion of the terminal carbons is, independently at each occurrence, a carbonyl carbon of an aldehyde group, a ketone group, an acid group, an acetate group, or the like. Methods of chain end analysis are known in the art. Non-limiting examples of chain end analysis include ¹H and ¹³C NMR, mass spectrometry, IR spectroscopy, Raman spectrometry, MALDI-TOF, and the like). In various examples, the chain end(s) of a cationic polymer are determined by one or more of these chain end analys(es).

A cationic polymer can have various molecular weights and/or polydispersities. In various examples, a cationic polymer has a molecular weight (Mw and/or Mn) of about 500 g/mol to about 1,000,000 g/mol, including all 0.1 g/mol values and ranges therebetween (e.g., about 30,000 g/mol to about 200,000 g/mol) and/or a polydispersity index (PDI) of about 1.05 or less to about 10 more, including all 0.01 values and ranges therebetween (e.g., 1.05 to 1.5). Molecular weight (Mw and/or Mn) and/or PDI can be determined by methods known in the art. In various examples, cationic polymer molecular weight (Mw and/or Mn) and/or PDI is determined by size exclusion chromatography (SEC), gel permeation chromatography (GPC), or the like, or any combination thereof.

A cationic polymer can have various forms. In various examples, a cationic polymer is in the form of a coating, a monolith, a film, a thin film, a sheet, a membrane, a pellet or pellets, a fiber, a powder, or the like.

In an aspect, the present disclosure provides methods of making cationic polymers. In various examples, a method results in formation of a cationic polymer or cationic polymer(s) of the present disclosure. In various examples, a method comprises a direct insertion polymerization or the like. Non-limiting examples of methods are described herein.

In various examples, a method of making a cationic polymer (e.g., a cationic polymer of the present disclosure, such as, for example, a polymer of any one of Statements 5-9, or the like) comprises: polymerizing one or more cationic monomer(s) independently chosen from cationic monomer(s) of the present disclosure (such as, for examples, a cationic monomer of any one of claims 1-4), where the polymerization is a direct insertion polymerization, and optionally, one or more crosslinker(s) (such as, for example, cationic crosslinker(s), one or more charge neutral crosslinking monomer(s), non-ionic crosslinking monomer(s), or the like, or any combination thereof); or copolymerizing one or more monomer(s) independently chosen from cationic monomer(s) of the present disclosure (such as, for examples, a cationic monomer of any one of Statements 1-4) and one or more co-monomer(s) (such as, for example, monomers other than cationic monomer(s) of the present disclosure (such as, for examples, a cationic monomer of any one of claims 1-4), monomers that do not comprise a cationic group, and the like), and optionally, one or more crosslinker(s) one or more crosslinker(s) (such as, for example, cationic crosslinker(s), one or more charge neutral crosslinking monomer(s), non-ionic crosslinking monomer(s), or the like, or any combination thereof), where the polymerization is a direct insertion polymerization. In various examples, a method is carried out in air or an inert atmosphere. In various examples, a method does not comprise any post-polymerization introduction of cationic group(s) (e.g., functionalization of a cationic polymer or the like).

In various examples, a method comprises forming a polymerization mixture. In various examples, a polymerization mixture comprises or consists essentially of the polymerization components (such as, for example the cationic monomer(s), optionally, one or more charge neutral or non-ionic crosslinking monomer(s), one or more catalyst(s) and/or activator(s), optionally, one or more solvent(s), and optionally, one or more salt(s) independently comprising one or more weakly coordinating anion(s), and, optionally one or more solvent(s).

In various examples, the polymerizing or copolymerizing comprises: forming a polymerization mixture comprising: the cationic monomer(s), optionally, one or more charge neutral or non-ionic crosslinking monomer(s), one or more catalyst(s) and/or activator(s), optionally, one or more solvent(s), and optionally, one or more salt(s) independently comprising one or more weakly coordinating anion(s); and holding the reaction mixture (e.g., for a time and at a temperature), where the cationic polymer is formed.

Various cationic monomers can be used. Combinations of cationic monomers can be used. Non-limiting examples of cationic monomers include multifunctional cationic monomers, cationic crosslinking monomers, and the like, and any combination thereof. In various examples, a monomer is an endo isomer, an exo isomer, or any combination thereof. In various examples, a polymerization mixture comprises a combination of at least two or more structurally different cationic monomer(s). In various examples, a polymerization mixture comprises one or more multifunctional monomer(s) and/or one or more cationic crosslinking monomer(s). In various examples, the only cationic monomer(s) in a polymerization mixture are multifunctional cationic monomers and/or cationic crosslinking monomers.

In various examples, at least a portion or all of the cationic monomers comprise (or at least a portion or all of the anions present in the polymerization mixture are weakly coordinating anions (such as, for example, BF₄⁻, SbF₆⁻, SbCl₆⁻, PF₆⁻, B(Ar)₄⁻, where Ar is an aryl group, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), NTf₂⁻, where Tf is a triflate group, or the like or any combination thereof). In various examples, a weakly coordinating anion coordinates to a cationic group substantially as strong or less strongly than a BF₄⁻ anion.

In various examples, a polymerization mixture does not comprise substantially any (e.g., less than 1 wt % (based on the total weight of the polymerization mixture, a detectible amount, or the like) strongly coordinating ions. In various examples, a polymerization mixture does not comprise any strongly coordinating anions. Non-limiting examples of strongly coordinating anions include halides (such as, for example, chloride, bromide, iodide, and the like) and the like and any combination thereof. In various examples, a strongly coordinating anion coordinates to a cationic group more strongly than a BF₄⁻ anion (such as, for example, chloride anion, bromide anion, iodide anion, carboxylate anion, or alkoxide anion).

Various co-monomers can be used. Combinations of at least two or more different co-monomers (e.g., structurally different co-monomers or the like) can be used. In various examples, one or more or all the co-monomer(s) is/are chosen from unsaturated hydrocarbon monomers.

In various examples, one or more neutral crosslinking monomer(s) and/or one or more or non-ionic crosslinking monomer(s) and/or one or more cationic crosslinking monomer(s) are polymerized or copolymerized (e.g., a polymerization mixture comprises one or more neutral crosslinking monomer(s) and/or one or more or non-ionic crosslinking monomer(s) and/or one or more cationic crosslinking monomer(s)). In various examples, at least a portion of or all of the cationic monomer(s) (or monomer(s)) is/are cationic crosslinking monomer(s).

In various examples, neutral or non-ionic crosslinking monomer(s) is/are chosen from:

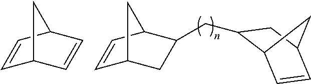

and structural analogs, thereof, and any combination thereof. In various examples, n is independently 1-20, including all integer n values and ranges therebetween. In various examples, cationic crosslinking monomer(s) is/are chosen from:

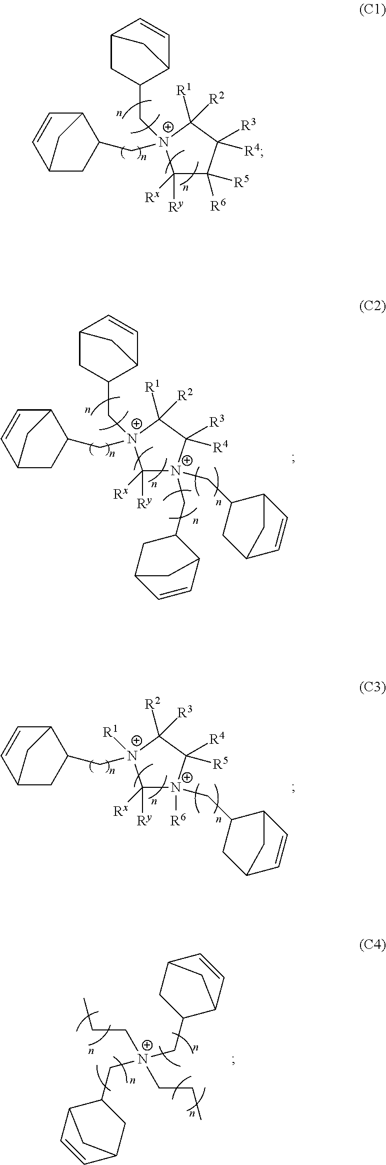

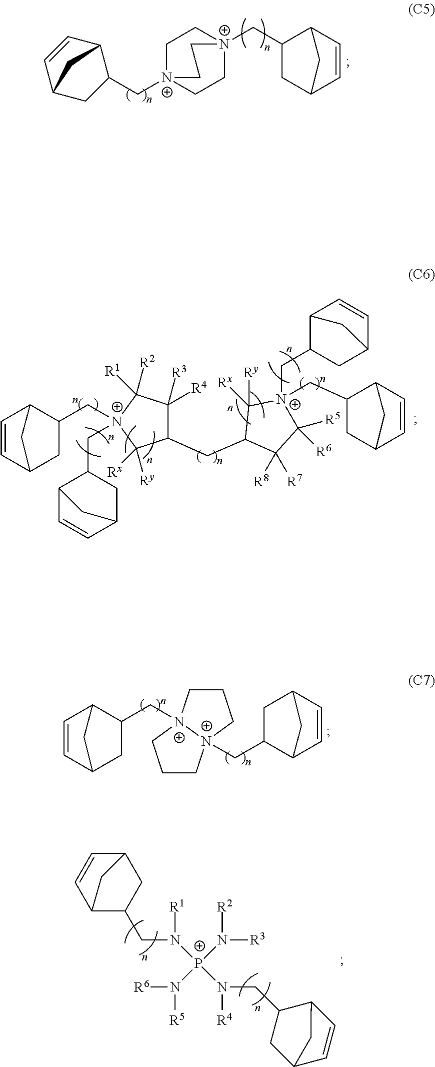

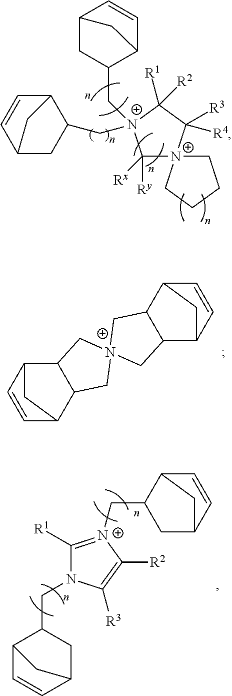

and structural analogs thereof, and any combination thereof. In various examples, the R groups (R^{x}, R^{y}, R¹, R¹, R¹, R¹, R¹, R¹, R¹, or R⁸, if present) are independently for each ionic crosslinking monomer chosen from hydrogen (H) group, alkyl groups, halide groups (chloride, bromide, iodide, or the like), ether groups, acyl groups, and the like. In various examples, n is independently 1-20, including all integer n values and ranges therebetween. An ionic comonomer may be a salt. In various examples, the anion(s) of the salt is/are chosen from weakly coordinating anions. In various example, the anion(s) of the salt is/are independently chosen from BF₄⁻, SbF₆⁻, SbCl₆⁻, PF₆⁻, B(Ar)₄⁻, where Ar is an aryl group, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), NTf₂⁻, where Tf is a triflate group, or the like or any combination thereof.

In various examples, the monomer(s) in the polymerization mixture comprise about 0.1 to about 100 mol % cationic crosslinking monomer(s) (based on the total moles of cationic monomers or all monomers present in the polymerization mixture), including all 0.1 mol % values and ranges therebetween. In various examples, the monomer(s) in the polymerization mixture comprise about 20 mol % or less, about 0.1 to about 20 mol %, less than about 50 mol %, or at least about 50 mol % cationic crosslinking monomer(s) (based on the total moles of cationic monomers or all monomers present in the polymerization mixture), including all 0.1 mol % values and ranges therebetween.

Suitable catalysts and activators are known in the art. Non-limiting examples of catalysts and activators are found in Blank and Janiak “Metal catalysts for the vinyl/addition polymerization of norbornene” Coordination Chemistry Reviews, Volume 253, Issues 7-8, 2009; and Janiak, and Lassahn “The Vinyl Homopolymerization of Norbornene. Macromol. Rapid Commun., (2001) 22: 479-493, the disclosure of which with regard to catalysts and activators is incorporated herein by reference. In various examples, a catalyst is an air stable catalyst.

Non-limiting examples of catalysts are provided herein. In various examples, a catalyst comprises titanium, zirconium, chromium, iron, cobalt, nickel, palladium, or copper, or the like, or any combination thereof. In various examples, a catalyst is a titanium catalyst, a chromium catalyst, an iron catalyst, a cobalt catalyst, a nickel catalyst, a palladium catalyst, or a copper catalyst, or the like, or any combination thereof. Other non-limiting examples catalysts and activators include chloro(tri-tert-butylphosphine)methylpalladium, chloro(triadamantylphosphine)methylpalladium, bis(1,5-cyclooctadiene) nickel, and the like, and any combination thereof.

In various examples, an activator is a salt. In various examples, an activator is a SbF₆ salt, a BF₄ salt, a PF₆ salt, a Ar^{F} salt (where Ar^{F} is an aryl group substituted with one, two, three, four, or five fluorine group(s) or one, two, three, four, or five fluoroalkyl group(s) or a Li salt, sodium salt, a potassium salt, a silver salt, or the like, thereof. Non-limiting examples of activators include LiSbF₆, KSbF₆, AgSbF₆, NaSbF₆, LiBF₄, KBF₄, AgBF₄, NaBF₄, LiPF₆, KPF₆, AgPF₆, NaPF₆, LiB(Ar^{F})₄, KB(Ar^{F})₄, AgB(Ar^{F})₄, NaB(Ar^{F})₄, where Ar^{F} is an aryl group substituted with one, two, three, four, or five fluorine group(s) or one, two, three, four, or five fluoroalkyl group(s), structural analogs thereof, and any combination thereof.

In various examples, a polymerization mixture further comprises one or more solvent(s). Non-limiting examples of solvents include hydrocarbon solvents (which may be chlorinated hydrocarbon solvents) such as, for example, dichloromethane, chloroform, dichloroethane, tetrachloroethane, chlorobenzene, toluene, trifluorotoluene, and the like and any combination thereof. Non-limiting examples of solvents include polar aprotic solvents, polar protic solvents (such as, for example, alcohols (e.g., methanol, ethanol, propanol, butanol, and the like), and the like).

A polymerization reaction can be performed under various reaction conditions. A polymerization reaction can comprise one or more step(s) and each step can be performed under the same or different reaction conditions as other steps.

A polymerization reaction can be carried out at various temperatures. In various examples, a polymerization reaction is carried out at about room temperature (e.g., from about 20° C. to about 30° C., including all 0.1° C. values and ranges therebetween), below room temperature (e.g., below about room temperature, such as for example, from about −78° C. to about room temperature, including all 0.1° C. values and ranges therebetween), or above room temperature (e.g., above room temperature up to or about a boiling point of the solvent(s), if present) (e.g., room temperature to about 100° C. or above, or any combination thereof (e.g., where each polymerization is performed at a different temperature as other steps). In various examples, a polymerization reaction is carried out at about −78° C., to about 100° C., including all 0.1° C. values and ranges therebetween.

A polymerization reaction can be carried out at various pressures. In various examples, a polymerization reaction is carried out at atmospheric pressure (e.g., 1 standard atmosphere (atm) at sea level), at greater than atmospheric pressure (e.g. heating in a sealed pressurized reaction vessel and the like), at below atmospheric pressure (e.g., under vacuum (e.g., from about 1 mTorr or less to about 100 mTorr or less, including all 0.1 mTorr values and ranges therebetween) (e.g., about 100 mTorr or less, about 50 mTorr or less, about 10 mTorr or less, or about 1 mTorr or less) and the like), or any combination thereof (e.g., where each step is performed at a different pressure as other steps).

A polymerization reaction can be carried out for various times. The reaction time can depend on factors such as, for example, temperature, pressure, presence and/or efficiency of the catalyst(s)/activator(s), presence and/or intensity of an applied energy source, mixing (e.g., stirring or the like), monomer(s) used, or the like, or a combination thereof. In various examples, reaction times range from about minutes (e.g., 1 minute) to greater than about 24 hours, including all integer second values and ranges therebetween, or any combination thereof (e.g., where each step is performed at a different time as other steps).

In various examples, a method further comprising isolating the cationic polymer or mixture of cationic polymers. Suitable polymer isolation methods are known in the art. Non-limiting examples, of isolation methods include filtration, centrifugation, precipitation, chromatography, or the like, or any combination thereof.

In various examples, a cationic polymer is in the form of a coating, a monolith, a film, a thin film, a sheet, a membrane, a pellet or pellets, a fiber, a powder, or the like. In various examples, a method further comprises processing a cationic polymer to form a coating, a monolith, a film, a thin film, a sheet, a membrane, a pellet or pellets, a fiber, a powder, or the like.

In an aspect, the present disclosure provides anion exchange membranes. In various examples, an anion exchange membrane comprises one or more cationic polymer(s) of the present disclosure (e.g., cationic polymer(s) according to any one of Statement 5-9, polymer(s) made by a method according to any one of Statements 10-15, or the like, or the like). Non-limiting examples of anion-exchange membranes are described herein.

An anion exchange membrane can be used in various devices. Non-limiting examples of devices are provided herein.

An anion exchange membrane can have various thicknesses. In various examples, an anion exchange membrane comprises a thickness suitable for use in a device described herein. In various examples, an anion exchange membrane independently comprises a thickness of about 1 micron to about 500 microns, including all 0.1 micron values and ranges therebetween.

An anion exchange membrane can exhibit various desirable properties. In various examples, an anion exchange membrane exhibits: a hydroxide conductivity (σ(OH−, at about 22° C.)) of from about 1 mS/cm to about 60 mS/cm, including all 0.1 mS/cm values and ranges therebetween; a water uptake (WU) of from about 5% to about 200%, including all 0.1% values and ranges therebetween; a dimensional change (ΔL) of from about 0% to about 30%, including all 0.1% values and ranges therebetween; an ion exchange capacities (IECs) of from about 0.005 mmol I⁻/g to about 4 mmol I⁻/g, including all 0.1 mmol I⁻/g values and ranges therebetween; or a retained conductivity of from about 0% to about 100%, including all 0.1% values and ranges therebetween; or any combination thereof.

An anionic exchange membrane (or a cationic polymer) can exhibit desirable stability. In various examples, an anionic exchange membrane (or a cationic polymer) exhibits no significant or no loss in hydroxide conductivity (σ(OH−, at about 22° C.)) and/or no significant mass loss or no mass loss after 30 days, or 45 days, or 60 days in about 1M KOH(aq.) at about 80° C. In various examples, an anionic exchange membrane (or a cationic polymer) exhibits a 10% or less, 5% or less, 1% or less, 0.1% or less decrease in hydroxide conductivity (σ(OH−, at about 22° C.)) and/or mass loss after 30 days, or 45 days, or 60 days in about 1M KOH(aq.) at about 80° C.

An anion exchange membrane can be formed by various methods. Suitable methods are known in the art. In various examples, an anion exchange membrane is formed by a method comprising crosslinking/reactive casting, solution casting, annealing, meltpressing, or the like.

In an aspect, the present disclosure provides devices. In various examples, a device comprises one or more cationic polymer(s) of the present disclosure (e.g., cationic polymer(s) according to any one of claims 5-9, polymer(s) made by a method according to any one of Statements 10-15, or the like, or the like). In various examples, a device comprises one or more anion exchange membrane(s) of the present disclosure. (e.g., anion exchange membrane(s) according to any one of Statements 16-19 or the like) Non-limiting examples of devices are described herein.

Suitable devices in which an anion exchange membrane can be used are known in the art. In various examples, a device is an electrochemical device or the like. In various examples, a device is a sensor, an actuator an energy-storage device, an energy-generating device, or the like, or any combination thereof. In various examples, an actuator or sensor is a biomimetic actuator or sensor or the like. In various examples, an electrochemical device is a battery, a fuel cell, a water-electrolysis device, a water electrolyzer, an electrodialysis device, or the like.

In an aspect, the present disclosure provides uses of cationic compounds and polymers of the present disclosure. Non-limiting examples of uses are described herein.

In various examples, one or more cationic compound(s) is/are used as monomer(s) in a polymerization reaction. In various examples, one or more cationic compound(s) is/are used as monomer(s) in a polymerization reaction of the present disclosure or the like.

In various examples, one or more cationic compound(s) is/are used as materials for gene/drug delivery or the like. In various examples, one or more cationic compound(s) are used as (or is/are) anti-fouling agents and/or antimicrobial agents, or the like.

The following Statements describe various examples of compositions and of the present disclosure that are not intended to be limiting in any manner:
Statement 1. A compound comprising (or having) one or more norbornenyl group(s) and at least one cationic group independently at each occurrence chosen from phosphonium groups (such as, for example, acyclic phosphonium groups and cyclic phosphonium groups), imidazolium groups, cyclic ammonium groups (e.g., where the cyclic ammonium groups are covalently linked via a linking group to the norbornene ring of a norbornenyl group), and the like, where each cationic group is covalently linked (e.g., directly linked (such as, for example, fused to) or linked via a linking group) to the norbornene ring of a norbornenyl group.
Statement 2. A compound according to Statement 1, the compound comprising (or having) the following structure:

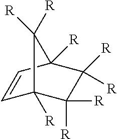

and structural analogs thereof, or a salt, a partial salt, a solvate, a polymorph, or the like thereof, or a stereoisomer or a mixture of stereoisomers, an isotopic variant, a tautomer, or the like thereof, where R is independently at each occurrence chosen from H group, alkyl group (e.g., C₁-C₂₀ alkyl group, including all integer number of carbons therebetween), cationic group (e.g., cationic group covalently linked to the norbornene ring) chosen from phosphonium groups, imidazolium groups, cyclic ammonium groups and the like, and the like, with the proviso that at least one R group is a cationic group (e.g., cationic group covalently linked to the norbornene ring via a linking group).
Statement 3. A compound according to Statement 1 or Statement 2, where the compound comprises the following structure:

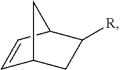

or a structural analog thereof, or the like.
Statement 4. A compound according to any one of the preceding Statements, where the compound further comprises one or more other cationic group(s) (e.g., a quaternary cationic group comprising (or having) the following structure:

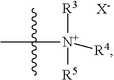

where R¹, R², and R³ are independently at each occurrence an aliphatic or aryl group (such as, for example, a C₁, C₂, C₃, C₄, C₅, and C₆ alkyl groups and the like)) independently chosen from quaternary ammonium groups (such as, for example, acyclic quaternary ammonium groups and the like) and the like.
Statement 5. A polymer (e.g., a homopolymer or a copolymer), which is also referred to herein as a cationic polymer, comprising the following structure:

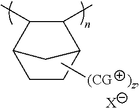

where
CG is cationic group (e.g., a cationic group covalently linked to the norbornyl group (such as, for example a non-backbone carbon) via a linking group, a cationic group fused to the norbornyl group, or the like) independently chosen from phosphonium groups, imidazolium groups, cyclic ammonium groups, and the like.
X⁻ is an anion (such as, for example, a halide anion (e.g., F⁻, Cl⁻, Br⁻— or I⁻) or a complex anion (such as, for example, BF₄⁻, SbF₆⁻, SbCl₆⁻, PF₆⁻, B(Ar^{F})₄⁻, where Ar^{F} is an aryl group substituted with one, two, three, four, or five fluorine group(s) or one, two, three, four, or five fluoroalkyl group(s) (such as, for example, trifluoromethyl groups or the like), B(Ar^{F})₄⁻, where Ar is an aryl group, and NTf₂⁻, here Tf is a triflate group, and the like) or the like),
z is independently at each occurrence 1, 2, 3, 4, or 5, and n is 1 to 10,000, including all integer n values and ranges therebetween (e.g., 2 to 10,000).
Statement 6. A polymer according to Statement 5, where at least a portion or all of the repeat units further comprise one or more other cationic group(s) (OCG(s)) independently chosen from quaternary ammonium groups and the like.
Statement 7. A polymer according to Statement 5 or 6, where the polymer has a molecular weight (Mw and/or Mn) of about 500 g/mol to about 1,000,000 g/mol, including all 0.1 g/mol values and ranges therebetween (e.g., about 30,000 g/mol to about 200,000 g/mol).
Statement 8. A polymer according to any one of Statements 5-7, where the polymer is a copolymer, and the polymer further comprises one or more repeat unit(s) independently at each occurrence chosen from

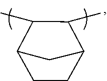

structural analogs thereof, and the like.
Statement 9. A polymer according to any one of Statements 5-8, where the polymer is crosslinkable or crosslinked (e.g., intramolecularly crosslinked, intermolecularly crosslinked, or both).
Statement 10. A method of making a polymer (e.g., a polymer of the present disclosure, such as, for example, a polymer of any one of Statements 5-9, or the like) comprising following: polymerizing one or more monomer(s) independently chosen from cationic monomer(s) of the present disclosure (such as, for examples, a cationic monomer of any one of Statements 1-4), where the polymerization is a direct insertion polymerization, or copolymerizing one or more monomer(s) independently chosen from cationic monomer(s) of the present disclosure (such as, for examples, a cationic monomer of any one of Statements 1-4) and one or more co-monomer(s) (such as, for example, monomers other than cationic monomer(s) of the present disclosure (such as, for examples, a cationic monomer of any one of Statements 1-4), monomers that do not comprise a cationic group, and the like), where the polymerization is a direct insertion polymerization.
Statement 11. A method according to Statement 11, where the polymerizing or copolymerizing comprises: forming a polymerization mixture comprising: the monomer(s), optionally, one or more charge neutral or non-ionic crosslinking monomer(s), one or more catalyst(s), optionally, one or more solvent(s), optionally, one or more activator(s), and optionally, one or more salt(s) independently comprising one or more weakly coordinating anion(s); and holding the reaction mixture (e.g., for a time and at a temperature), where the polymer is formed.
Statement 12. A method according to Statement 10 or 11, where at least a portion of or all of the cationic monomer(s) (or monomer(s)) is/are cationic crosslinking monomer(s).
Statement 13. A method according to any one of Statements 10-12, where the polymerization mixture further comprises one or more solvent(s).
Statement 14. A method according to any one of Statements 10-13, the method further comprising isolating the polymer.
Statement 15. A method according to Statement 14, where at least a portion or all of the polymer is isolated by filtration, centrifugation, precipitation, chromatography, or the like, or any combination thereof.
Statement 16. An anion exchange membrane comprising one or more polymer(s) of the present disclosure (e.g., polymer(s) according to any one of Statements 5-9, polymer(s) made by a method according to any one of Statements 10-15, or the like).
Statement 17. An anion exchange membrane according to Statement 16, where the membrane has a thickness of about 1 micron to about 500 microns, including all 0.1 micron values and ranges therebetween.
Statement 18. An anion exchange membrane according to Statement 16 or 17, where the membrane exhibits one or more or all of the following: a hydroxide conductivity (σ(OH−, at about 22° C.)) of from about 1 mS/cm to about 60 mS/cm; a water uptake (WU) of from about 5% to about 200%; a dimensional change (ΔL) of from about 0% to about 30%; an ion exchange capacities (IECs) of from about 0.005 mmol I⁻/g to about 4 mmol I⁻/g; a retained conductivity of from about 0% to about 100%.
Statement 19. An anion exchange membrane according to any one of Statements 16-18, where the membrane is formed by a method comprising crosslinking/reactive casting, solution casting, annealing, meltpressing, or the like.
Statement 20. A device comprising one or more anion exchange membrane(s) of the present disclosure (e.g., anion exchange membrane(s) according to any one of Statements 16-19 or the like).
Statement 21. A device according to Statement 20, where the device is a sensor, an actuator an energy-storage device, an energy-generating device, or the like.
Statement 22. A device according to Statement 20 or 21, where the electrochemical device is a battery, a fuel cell, a water-electrolysis device, an electrodialysis device, or the like.
Statement 23. A cationic compound comprising one or more norbornenyl group(s) and at least one cationic group, where each cationic group is independently at each occurrence chosen from phosphonium groups, imidazolium groups, cyclic ammonium groups, and the like, and where each cationic group is covalently linked or covalently linked via a linking group to the norbornene ring of a norbornenyl group.
Statement 24. A cationic compound according to Statement 23, where each cationic group is directly covalently linked to the norbornene ring or covalently linked via a linking group via a carbon other than a carbon of the carbon-carbon double bond of the norbornenyl group or each cationic group is covalently linked to the norbornene ring via a carbon at the endo or exo position other than a carbon of the carbon-carbon double bond of the norbornenyl group.
Statement 25. A cationic compound according to Statement 23 or 24, where each phosphonium group independently comprises the following structure:

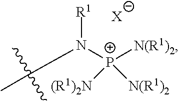

or a structural analog thereof, where R¹ is independently at each occurrence chosen from H group, alkyl groups, cyclic alkyl groups, aryl groups, and the like; and
X⁻ is an anion or a complex anion.
Statement 26. A cationic compound according to any one of Statements 23 to 25, where each imidazolium group independently comprises the following structure:

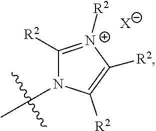

or a structural analog thereof, where R² is independently at each occurrence chosen from H group, alkyl groups, cyclic alkyl groups, and aryl groups, and the like; and X⁻ is an anion or a complex anion.
Statement 27. A cationic compound according to any one of Statements 23 to 26, where each cyclic ammonium group independently comprises the following structure:

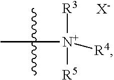

or a structural analog thereof, where (i) R³, R⁴, and the ammonium nitrogen (N⁺) taken together form a heterocyclic group where N⁺ is a member of the heterocyclic ring and R⁵ is an aliphatic or aryl group or the like, or (ii) R³, R⁴, R⁵, and N taken together form an aliphatic group-bridged heterocyclic group where N⁺ is a member of the heterocyclic ring; and X⁻ is an anion or a complex anion.
Statement 28. A cationic compound according to any one of Statements 23 to 27, where at least a portion or all of the cationic group(s) independently comprise a complex anion chosen from BF₄⁻, SbF₆⁻, SbCl₆⁻, PF₆⁻, B(Ar)₄⁻, where Ar is an aryl group, (Ar^{F})₄⁻, where Ar^{F} is an aryl group substituted with one, two, three, four, or five fluorine group(s) or one, two, three, four, or five fluoroalkyl group(s), and NTf₂⁻, where Tf is a triflate group, or the like.
Statement 29. A cationic compound according to any one of Statements 23 to 28, where the cationic compound comprises 2, 3, 4, or 5 cationic groups covalently linked to the norbornenyl group (e.g., directly or via a linking group).
Statement 30. A cationic compound according to any one of Statements 23 to 29, where the cationic compound comprises at least two cationic groups covalently linked directly to each other via a linking group.
Statement 31. A cationic compound according to any one of Statements 23 to 30, where the cationic compound comprises at least two cationic groups, where a first cationic group is covalently linked to the norbornenyl group and one or more second cationic groups(s) covalently linked to the first cationic group, where at least one of the cationic groups may be a phosphonium group or an imidazolium group or the like.
Statement 32. A cationic compound according to any one of Statements 23 to 31, where the cationic compound comprises two cationic groups linked directly to each other via an aliphatic group, a heterocyclic group, where at least one of the N atoms is quaternary, or a bridged heterocyclic group, where at least one of the N atoms is quaternary atom of a first quaternary ammonium group, or the like and an aliphatic group, a heterocyclic group, where at least one of the N atoms is quaternary, or a bridged heterocyclic group, where at least one of the N atoms is quaternary atom of a second quaternary, or the like.
Statement 33. A cationic compound according to any one of Statements 23 to 32, where the cationic compound comprises 2, 3, 4, or 5 norbornenyl groups covalently linked to a cationic group.
Statement 34. A cationic compound according to any one of Statements 23 to 33, where the cationic compound comprises a cationic group and at least two norbornenyl groups, where each of the norbornenyl groups are covalently linked to the cationic group (e.g., directly or via a linking group).
Statement 35. A cationic compound according to any one of Statements 23 to 34, where the cationic compound comprises a cyclic cationic group fused to a norbornenyl group or the like.
Statement 36. A cationic compound according to any one of Statements 23 to 35, where the cationic compound comprises the following structure:

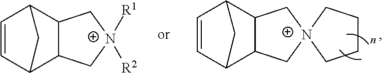

where n is 1, 2, 3, or 4, or the like.
Statement 37. A cationic compound according to any one of Statements 23 to 36, where the cationic compound comprises the following structure:

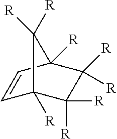

or a structural analog thereof, or a salt, a partial salt, a solvate, a polymorph, or the like thereof, or a stereoisomer or a mixture of stereoisomers, an isotopic variant, a tautomer, or the like thereof, where R is independently at each occurrence chosen from H group, alkyl group, cationic group chosen from phosphonium groups, imidazolium groups, and cyclic ammonium groups, and the like, with the proviso that at least one R group is a cationic group.
Statement 38. A cationic compound according to any one of Statements 23 to 37, where the cationic compound comprises the following structure:

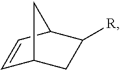

or a structural analog thereof, or a structural analog thereof, or a salt, a partial salt, a solvate, a polymorph, or the like thereof, or a stereoisomer or a mixture of stereoisomers, an isotopic variant, a tautomer, or the like thereof.
Statement 39. A cationic compound according to any one of Statements 23 to 38, where the R group is independently in the endo or exo position.
Statement 40. A cationic compound according to any one of Statements 23 to 39, where the cationic compound further comprises one or more other cationic group(s).
Statement 41. A cationic compound according to any one of Statements 23 to 40, where the cationic compound comprises the following structure:

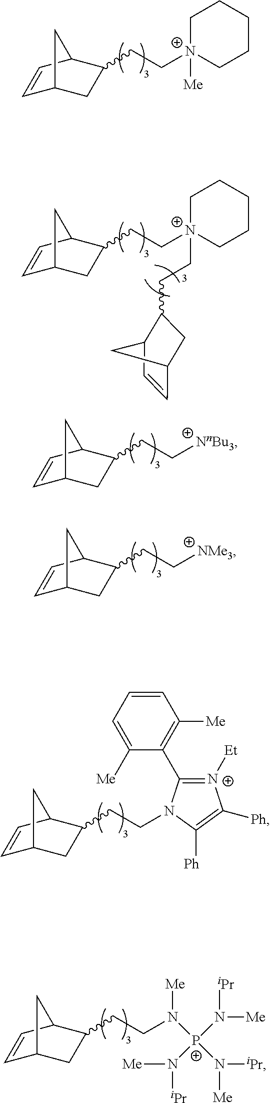

or a structural analog thereof.
Statement 42. A cationic polymer comprising the following repeat unit structure:

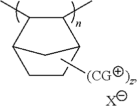

where CG is cationic group independently at each occurrence chosen from phosphonium groups, imidazolium groups, cyclic ammonium groups, where the cationic group(s) is/are independently at each occurrence directly covalently linked to the norbornyl group or covalently linked to the norbornyl group via a linking group, X⁻ is an anion or a complex anion, z is independently at each occurrence 1, 2, 3, 4, or 5, and n is 1 to 10,000, including all integer n values and ranges therebetween.
Statement 43. A cationic polymer according to Statement 42, where one or more cationic polymer repeat unit(s) comprises a cyclic cationic group fused to a norbornyl group.
Statement 44. A cationic polymer according to Statement 42 or 43, where at least a portion or all of the repeat units further comprise one or more other cationic group(s) independently chosen from quaternary ammonium groups and the like.
Statement 45. A cationic polymer according to any one of Statements 42 to 44, where the cationic polymer does not comprise cationic group(s) formed by post-cationic polymerization introduction of cationic group(s) or the like.
Statement 46. A cationic polymer according to any one of Statements 42 to 45, where the cationic polymer has a molecular weight (Mw and/or Mn) of about 500 g/mol to about 1,000,000 g/mol, including all 0.1 g/mol values and ranges therebetween.
Statement 47. A cationic polymer according to any one of Statements 42 to 46, where the cationic polymer is a copolymer or the like.
Statement 48. A cationic polymer according to any one of Statements 42 to 47, where the cationic polymer is a copolymer, and the cationic polymer further comprises one or more repeat unit(s) independently at each occurrence chosen from

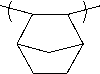

and the like and structural analogs thereof.
Statement 49. A cationic polymer according to any one of Statements 42 to 48, where the cationic polymer is crosslinkable or crosslinked.
Statement 50. A cationic polymer according to any one of Statements 42 to 49, where the cationic polymer is a network cationic polymer or the like.
Statement 51. A cationic polymer according to any one of Statements 42 to 50, where the cationic polymer or one or more domain(s) of a cationic polymer is/are amorphous or the like.
Statement 52. A method of making a cationic polymer of the present disclosure (e.g., a method of any one of Statements 42 to 51), the method comprising: polymerizing one or more cationic monomer(s) independently comprising one or more norbornenyl group(s) and at least one cationic group, where each cationic group is independently at each occurrence chosen from phosphonium groups, imidazolium groups, and cyclic ammonium groups, and the like, and where each cationic group is covalently linked or linked via a linking group to the norbornene ring of a norbornenyl group, where the polymerization is a direct insertion polymerization, or copolymerizing one or more cationic monomer(s) independently comprising one or more norbornenyl group(s) and at least one cationic group, where each cationic group is independently at each occurrence chosen from phosphonium groups, imidazolium groups, and cyclic ammonium groups, and the like, and where each cationic group is covalently linked or linked via a linking group to the norbornene ring of a norbornenyl group and one or more co-monomer(s) that do not comprise a cationic group, where the polymerization is a direct insertion polymerization.
Statement 53. A method according to Statement 52, where the polymerizing or copolymerizing comprises: forming a polymerization mixture comprising: the cationic monomer(s), optionally, one or more co-monomer(s), optionally, one or more charge neutral or non-ionic crosslinking monomer(s) or the like, one or more catalyst(s), optionally, one or more solvent(s), optionally, one or more activator(s), and optionally, one or more salt(s) independently comprising one or more weakly coordinating anion(s); and holding the reaction mixture (e.g., for a time and at a temperature), where the cationic polymer is formed.
Statement 54. A method according to Statement 52 or 53, where at least a portion or all of the cationic monomers comprise weakly coordinating ions.
Statement 55. A method according to any one of Statements 52 to 54, where a polymerization mixture comprises less than 1 wt % (based on the total weight of the polymerization mixture) of any strongly coordinating anions.
Statement 56. A method according to any one of Statements 52 to 55, where the catalyst(s) is/are chosen from chloro(tri-tert-butylphosphine)methylpalladium, chloro(triadamantylphosphine)methylpalladium, bis(1,5-cyclooctadiene) nickel, structural analogs thereof, and the like, and structural analogs thereof, and any combination thereof.
Statement 57. A method according to any one of Statements 52 to 56, where the activator(s) is/are chosen LiSbF₆, KSbF₆, AgSbF₆, NaSbF₆, LiBF₄, KBF₄, AgBF₄, NaBF₄, LiPF₆, KPF₆, AgPF₆, NaPF₆, LiB(Ar^{F})₄, KB(Ar^{F})₄, AgB(Ar^{F})₄, NaB(Ar^{F})₄, where Ar^{F} is an aryl group substituted with one, two, three, four, or five fluorine group(s) or one, two, three, four, or five fluoroalkyl group(s), and the like, and structural analogs thereof, and any combination thereof.
Statement 58. A method according to any one of Statements 52 to 57, where at least a portion of or all of the cationic monomer(s) (or monomer(s)) is/are cationic crosslinking monomer(s) and/or multifunctional cationic monomer(s).
Statement 59. A method according to any one of Statements 52 to 58, where the polymerization mixture comprises about 0.1 to about 100 mol % cationic crosslinking monomer(s) (based on the total moles of cationic monomers or all monomers present in the polymerization mixture), including all 0.01 mol % values and ranges therebetween.
Statement 60. A method according to any one of Statements 52 to 60, where the polymerization mixture further comprises one or more solvent(s).
Statement 61. A method according to any one of Statements 52 to 60, the method further comprising isolating the cationic polymer.
Statement 62. A method according to any one of Statements 52 to 61, where the method does not comprise any post-polymerization introduction of cationic group(s) or the like.
Statement 63. An anion exchange membrane comprising one or more cationic polymer(s) of the present disclosure (e.g., one or more cationic polymer(s) of any one of Statements 42 to 51 and/or one or more cationic polymer(s) made by a method of the present disclosure, such as, for example, one or more cationic polymer(s) independently made by a method of any one of Statements 52 to 62).
Statement 64. An anion exchange membrane according to Statement 63, where the membrane comprises a thickness of about 1 micron to about 500 microns, including all 0.1 micron values and ranges therebetween.
Statement 65. An anion exchange membrane according to Statement 63 or 64, where the membrane exhibits one or more or all of the following: a hydroxide conductivity (σ(OH−, at about 22° C.)) of from about 1 mS/cm to about 60 mS/cm; a water uptake (WU) of from about 5% to about 200%; a dimensional change (ΔL) of from about 0% to about 30%; an ion exchange capacities (IECs) of from about 0.005 mmol I⁻/g to about 4 mmol I⁻/g; or a retained conductivity of from about 0% to about 100%.
Statement 66. A device comprising one or more anion exchange membrane(s) of the present disclosure (e.g., one or more anion exchange membrane(s) of any one of Statements 63 to 65).
Statement 67. A device according to Statement 66, where the device is a sensor, an actuator, an energy-storage device, or an energy-generating device, or the like.
Statement 68. A device according to Statement 66 or 67, where the actuator is a biomimetic actuator or the like or the sensor is a biomimetic sensor or the like.
Statement 69. A device according to Statement 67, where the energy-storage device is a battery or the like or where the energy-generating device is a fuel cell, a water-electrolysis device, or an electrodialysis device, or the like.

The steps of the methods described in the various examples disclosed herein are sufficient to carry out a method of the present disclosure. Thus, in various examples, a method consists essentially of a combination of the steps of the methods disclosed herein. In various other examples, a method consists of such steps.

The following examples are presented to illustrate the present disclosure. They are not intended to be limiting in any manner.

### Example 1

This Example describes, inter alia, cationic compounds (which may be monomers), cationic polymers and uses thereof, and methods of making cationic polymers.

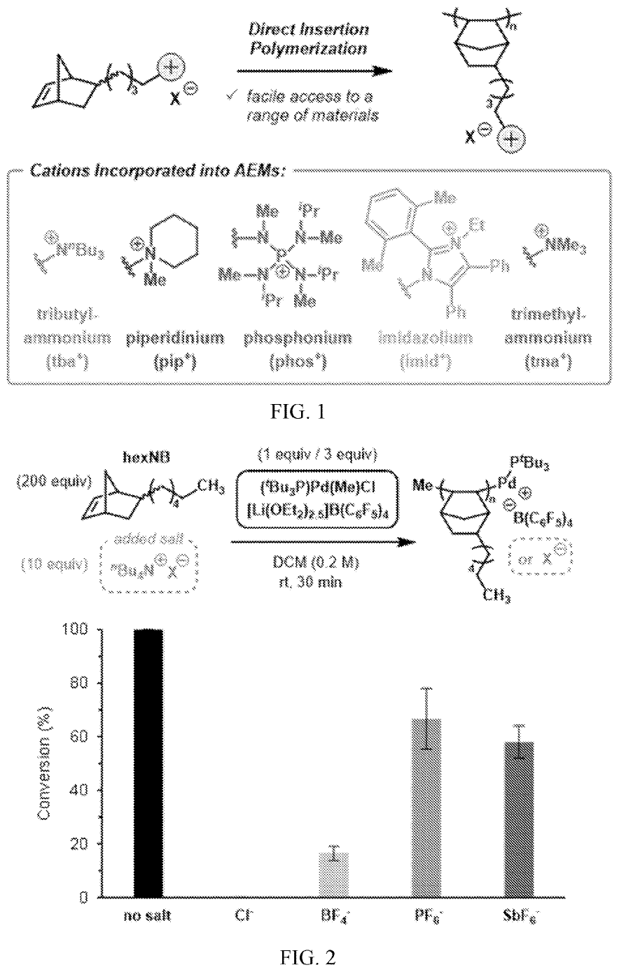

Direct Insertion Polymerization of Ionic Monomers: Rapid Production of Anion Exchange Membranes. An efficient and generally applicable direct coordination-insertion polymerization of ionic monomers (FIG. 1) was developed. Norbornene monomers containing tetrabutylammonium, piperidinium, phosphonium, imidazolium, and trimethylammonium cations all resulted in polymers with high ion incorporations. Importantly, this diverse library of aliphatic polymer-based AEMs was leveraged to study the influence of cation identity on hydroxide conductivity and stability. PNB membranes with piperidinium cations resulted in the highest hydroxide ion conductivity of 87 mS/cm at 80° C., and integration of this material into a working fuel cell device resulted in a peak power density of 730 mW/cm². This direct insertion polymerization provides a streamlined route to a broad range of ionic polymeric materials, and the following conductivity/stability study offers valuable guidance for the design of next-generation AEMs.

A direct coordination-insertion polymerization of cationic monomers, providing the first direct synthesis of aliphatic polymers with high ion incorporations and allowing facile access to a broad range of materials, is described. The utility of this method by rapidly generating a library of solution processable ionic polymers for use as AEMs was developed. These materials were investigated to study the influence of cation identity on hydroxide conductivity and stability. It was found that AEMs with piperidinium cations exhibited the highest performance, with high alkaline stability, hydroxide conductivity of 87 mS/cm at 80° C., and a peak power density of 730 mW/cm² when integrated into a fuel cell device.

Results and Discussion. Nozaki and co-workers reported a controlled insertion polymerization of norbornenes in 2006 using a ^{t}Bu₃P-ligated methylpalladium catalyst, which was shown in 2015 by Register and co-workers to tolerate substituted norbornenes. Whether or not this catalyst could be used to directly polymerize monomers with covalently bound cationic groups, where the identity of the monomer counteranion would determine the activity of the catalyst, was investigated. Thus, studies began by surveying the effect of different anions on the catalytic activity of (^{t}Bu₃P)Pd(Me)Cl with activator, [Li(OEt₂)_{2.5}]B(C₆F₅)₄.

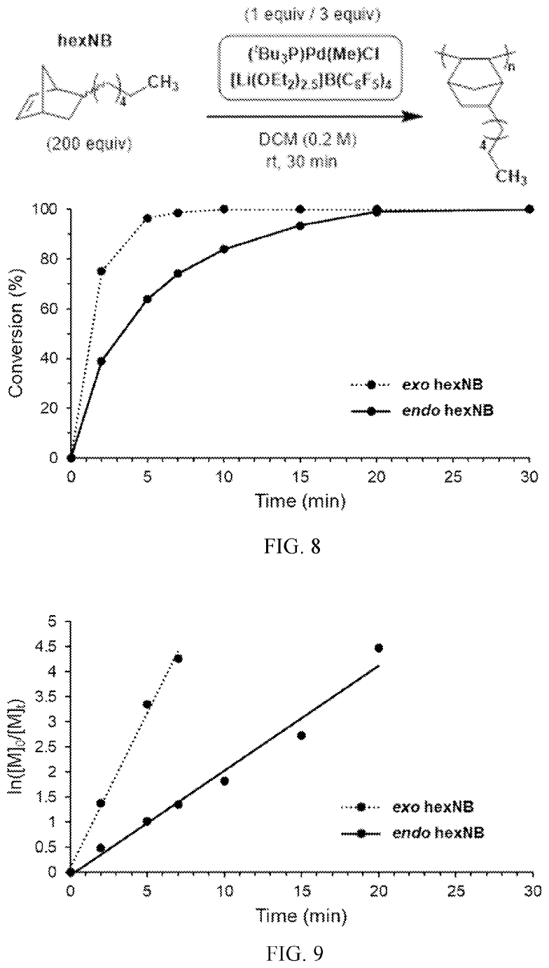

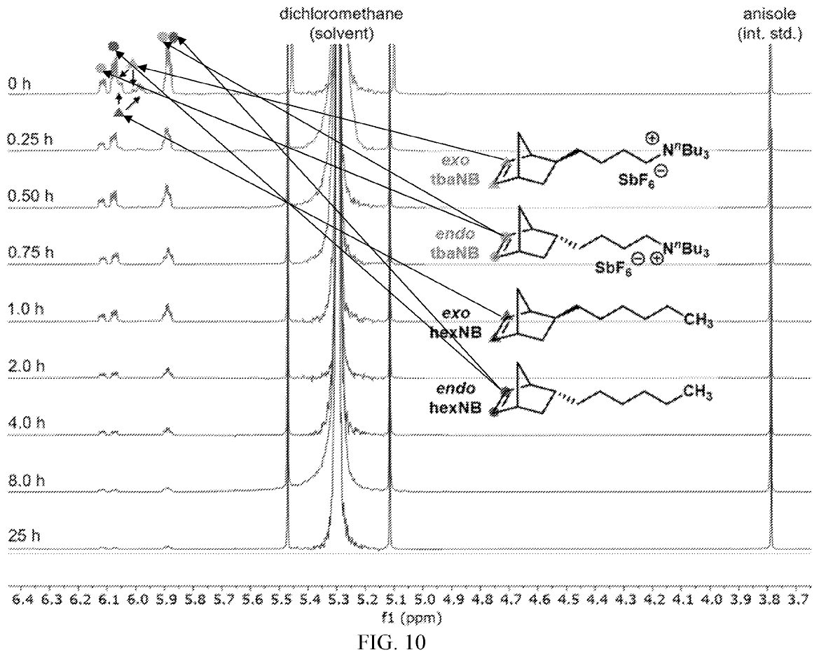

Hexyl-substituted norbornene (hexNB, 200 equivalents, 0.2 M) in dichloromethane (DCM) was polymerized in the absence and presence of tetrabutylammonium (TBA) salts with counteranions of varying coordination ability (FIG. 2). When no salt was added, the polymerization proceeded according to first-order kinetics with respect to monomer, reaching full conversion in 30 minutes (Table 1, entry 1, FIG. 8 and FIG. 9). When TBA chloride (Cl⁻) was added (10 equivalents relative to catalyst), no polymerization was observed, which was attributed to coordination and deactivation of the catalyst by chloride (Table 1, entry 2). In contrast, in the presence of TBA tetrafluoroborate (BF₄⁻), the polymerization reached 17% conversion after 30 minutes (Table 1, entry 3); and with TBA hexafluorophosphate (PF₆⁻) or TBA hexafluoroantimonate (SbF₆⁻) the polymerization reached approximately 60% conversion after 30 minutes (Table 1, entries 4 and 5). These results reflect the relative coordination abilities of Cl⁻, BF₄⁻, PF₆⁻, and SbF₆⁻, where higher conversions were achieved in the presence of more weakly coordinating anions. Moreover, polymerizations in the presence of the least coordinating anion, SbF₆⁻, exhibited the best match between theoretical molecular weight (Mₙ^{theo}) and experimental molecular weight (Mₙ^{exp}) and displayed the lowest dispersity value (Ð). These results demonstrate that high catalyst activity and a relatively controlled coordination-insertion polymerization can be achieved in the presence of an exogenous ion pair when the anion is weakly coordinating.

TABLE 1
Polymerization of hexyl-substituted norbornene (hexNB)
in the absence or presence of tetrabutylammonium
salts containing different counterions.

| | | | Mₙ^{theo} | Mₙ^{exp} | |
|---|---|---|---|---|---|
| Entry | Anion | Conversion^{[a]} | (kg/mol)^{[b]} | (kg/mol)^{[c]} | Ð^{[c]} |
| 1 | no salt | >99% | 35.7 | 27.4 | 1.18 |
| 2 | Cl⁻ | <5.0% | n.d. | n.d. | n.d. |
| 3 | BF₄⁻ | 17% | 5.88 | 3.47 | 3.33 |
| 4 | PF₆⁻ | 67% | 23.8 | 11.7 | 1.97 |
| 5 | SbF₆⁻ | 58% | 20.7 | 14.9 | 1.58 |

^{[a]}Determined by ¹H NMR.
^{[b]}Mₙ^{theo} = (Conv_{hexNB} × [hexNB]/[Pd] × MW_{hexNB}).
^{[c]}Determined by gel permeation chromatography relative to polystyrene standards.

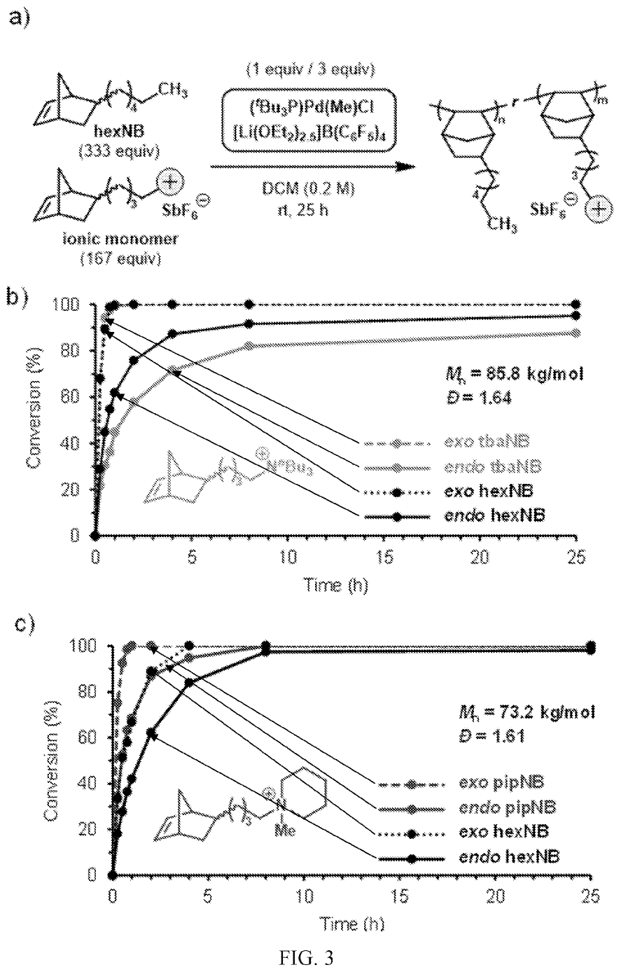

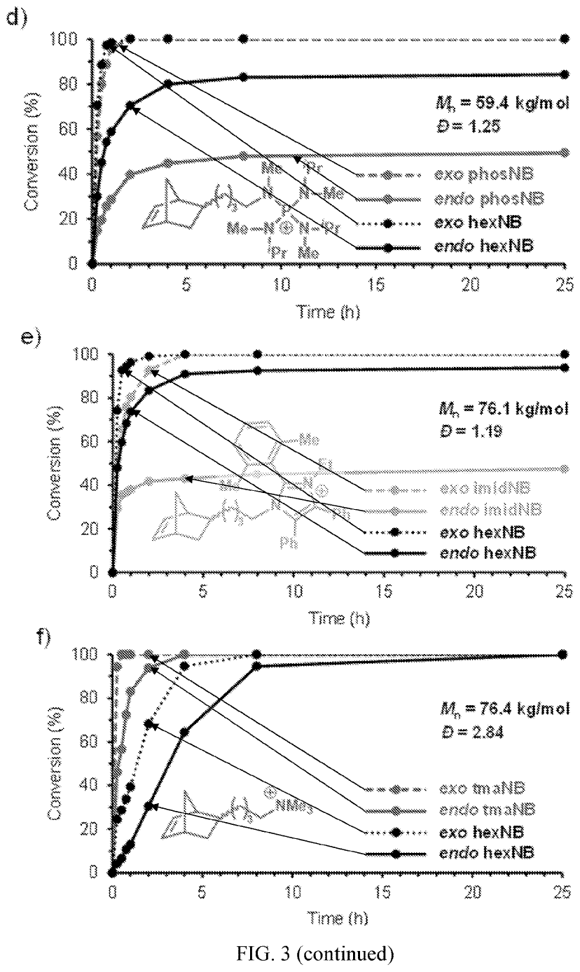

Having identified SbF₆⁻ as a promising anion, polymerization of a tetrabutylammonium-functionalized norbornene monomer (tbaNB) containing an SbF₆⁻ counterion was pursued. tbaNB, which exhibited an endo:exo ratio of approximately 80:20, was synthesized and the halide counterion exchanged for SbF₆⁻. It was determined this exchange was quantitative using DART-MS. It was chosen to copolymerize tbaNB with hexNB, as neutral comonomers are typically incorporated into AEMs to limit swelling and maintain mechanical integrity upon hydration of the material. Combining tbaNB and hexNB in a 1:2 ratio along with DCM, catalyst, and activator (FIG. 3a) resulted in efficient polymerization to 94% total monomer conversion within 25 hours (FIG. 3b, Table 2). The copolymerization achieved a high ionic monomer incorporation of 31 mol %, which was in close agreement with the monomer feed ratio of 32 mol %. This ion incorporation indicated a theoretical ion exchange capacity (IEC^{theo}) of 1.29 mmol Cl⁻/g, matching the value determined by ¹H NMR of the polymer (Table 2). Analysis by gel-permeation chromatography (GPC) revealed a Ð of 1.64 and an Mₙ^{exp} of 85.8 kg/mol, which was in good agreement with the Mₙ^{theo} of 107 kg/mol. In contrast, no polymerization with tbaNB containing a bromide counterion was observed. These results demonstrate that by exchanging the monomer counterion to a weakly coordinating anion such as SbF₆⁻, a direct coordination-insertion polymerization that reaches high ion incorporations can be achieved.

TABLE 2
Polymerization and characterization of AEMs synthesized via direct insertion polymerization.

| | Cation | Cation | | | | | IEC^{theo} | σ^{22° C.} | | | WU | | |
|---|---|---|---|---|---|---|---|---|---|---|---|---|---|
| | Feed | Incorp. | Total | Mₙ^{theo, b} | Mₙ^{exp, c} | | (mmol | (mS/ | σ^{80° C.} | σ^{80° C.}/ | (wt | | ΔL |
| Cation | (mol %)^{a} | (mol %)^{a} | Conv.^{a} | (kg/mol) | (kg/mol) | Ð^{c} | Cl⁻/g)^{d} | cm)^{e, j} | (mS/cm)^{e} | IEC^{theo} | %)^{f, j} | λ^{g} | (% )^{h, j} |
| tba⁺ | 32 | 31 | 94% | 107 | 85.8 | 1.64 | 1.29 | 22 | 42 | 33 | 54 | 23 | 10 |
| pip⁺ | 34 | 35 | 99% | 99.8 | 73.2 | 1.61 | 1.61 | 40 | 87 | 54 | 79 | 27 | 9 |
| phos⁺ | 36 | 28 | 78% | 94.6 | 59.4 | 1.25 | 1.09 | 22 | 41 | 38 | 46 | 23 | 12 |
| imid⁺ | 29 | 19 | 84% | 103 | 76.1 | 1.19 | 0.78 | 12 | 20 | 26 | 41 | 29 | n.d.ⁱ |
| tma⁺ | 26 | 26 | >99% | 94.2 | 76.4 | 2.84 | 1.31 | 29 | 53 | 40 | 58 | 25 | 10 |

^{a}Determined by ¹H NMR.
^{b}Mₙ^{theo} = (Conv_{catNB} × [catNB]/[Pd] × [MW_{catNB} − MW_{SbF6}]) + (Conv_{hexNB} × [hexNB]/[Pd] × MW_{hexNB}).
^{c}Determined by gel permeation chromatography relative to polystyrene standards.
^{d}Theoretical ion exchange capacity in chloride form determined from relative monomer conversions observed via ¹H NMR.
^{e}Determined using electrochemical impedance spectroscopy with a four-point probe.
^{f}Water uptake determined by the percent weight change of the membrane before and after drying, performed in triplicate.
^{g}Hydration number equaling the number of water molecules per cationic group in the membrane.
^{h}Swelling ratio determined by the percent change in length of the membrane before and after drying, performed in triplicate.
ⁱNot determined due to mechanical failure of the completely dried membrane.
^{j}Error values were omitted for clarity and are reported in Table 3.

TABLE 3
Measured hydroxide conductivity, water uptake,
and swelling ratios in all membranes.

| | σ^{22° C.} | WU | ΔL |
|---|---|---|---|
| Cation | (mS/cm)^{a} | (wt %)^{b} | (%)^{c} |
| tetrabutylammonium | 22 ± 3 | 54 ± 7 | 10 ± 1 |
| piperidinium | 40 ± 2 | 79 ± 5 | 9 ± 4 |
| phosphonium | 22 ± 2 | 46 ± 7 | 12 ± 2 |
| imidazolium | 11 ± 2 | 41 ± 8 | n.d.^{d} |
| trimethylammonium | 30 ± 3 | 58 ± 8 | 10 ± 3 |

^{a}Determined using electrochemical impedance spectroscopy with a four-point probe, performed in triplicate with error calculated to 95% confidence.
^{b}Water uptake (WU) determined by the percent weight change of the membrane, performed in triplicate with error calculated to 95% confidence.
^{c}Swelling ratio (ΔL) determined by the percent change in length of the membrane before and after drying, performed in triplicate with error calculated to 95% confidence.
^{d}Not determined due to mechanical failure of the completely dried membrane.

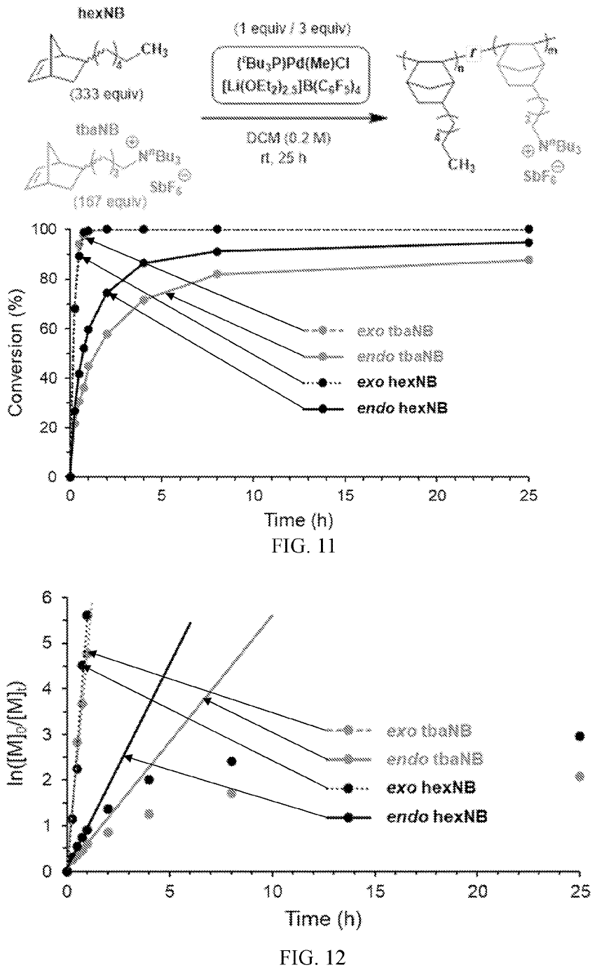

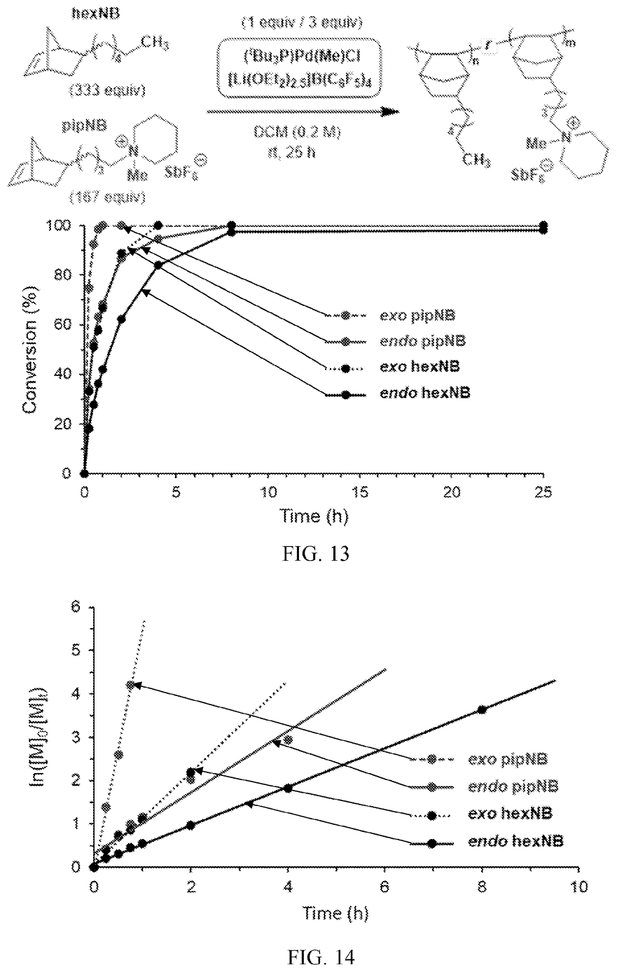

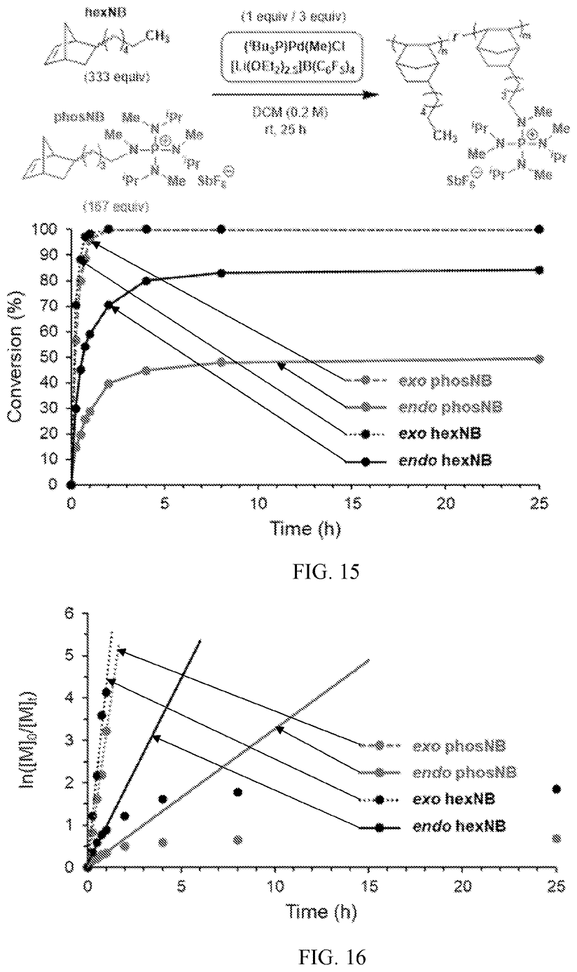

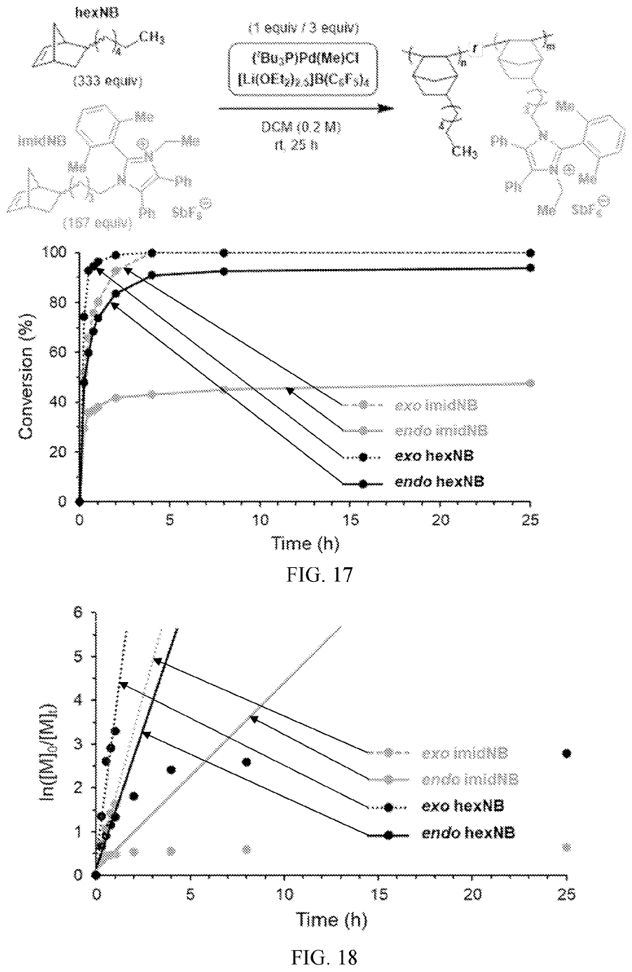

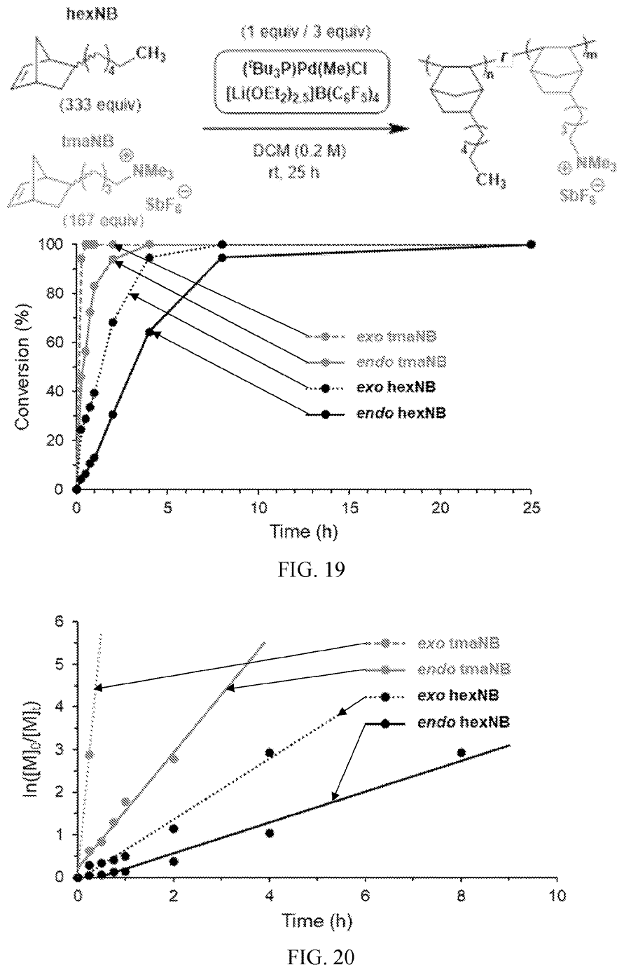

Kinetic studies showed that the exo isomers of both hexNB and tbaNB polymerized significantly faster than the endo isomers (FIG. 3b). This observation is consistent with previous reports which attribute this difference in reactivity to the greater steric restriction of the endo isomer. More interestingly, however, it was observed that the polymerization rate of the remaining endo isomers slowed drastically once the more reactive exo monomers were completely consumed (FIG. 12). It was hypothesized that this reduced ability of the remaining endo monomers to polymerize is due to the larger size of the pendent tetrabutylammonium substituent, as this stagnation in the homopolymerization of hexNB was not observed. It was hypothesized the reduction of endo tbaNB consumption is due to reactivity ratio differences arising from energetic differences in sequential exo-exo, exo-endo, and endo-exo monomer insertions relative to that of sequential endo-endo insertions, which ultimately allows heteropropagation but disallows homopropagation of endo tbaNB.

Similarly, Register and co-workers have reported a system in which the endo isomer of pentamethyldisiloxylnorbornene was unable to polymerize in the absence of the exo monomer. This example suggests that it is not the presence of cation, but rather, it is the presence of the sterically restricted endo isomer that hinders reactivity.

It was sought to expand the scope of this direct insertion polymerization and produce a library of polymers containing a range of different cations, obviating the need for post-polymerization modifications. Monomers with piperidinium (pipNB), phosphonium (phosNB), imidazolium (imidNB), and trimethylammonium (tmaNB) cations were selected. Following the procedure described above, norbornene monomers containing these cations were successfully polymerized, resulting in moderate to high monomer conversions within 25 hours (FIG. 3c-f). GPC analysis of all polymers revealed molecular weights ranging between 59 kg/mol and 76 kg/mol with good agreement between Mₙ^{exp} and Mₙ^{theo} (Table 2). Polymerizations of pipNB, imidNB and phosNB exhibited lower Ðs of 1.61, 1.19, and 1.25, respectively, whereas tmaNB resulted in a higher Ð of 2.84 which was attributed to the precipitation of polymer during the reaction.

The polymerization kinetics of monomers with bulkier cations, imidazolium (imidNB) and phosphonium (phosNB), resembled the kinetics discussed above with tbaNB, where the polymerization rate slowed drastically once the more reactive exo monomers were completely consumed (Table 2, FIG. 16 and FIG. 18). This effect limited total monomer conversions of imidNB and phosNB polymerizations to 84% and 78% and ion incorporations to 19 mol % and 28 mol %, respectively (Table 2). In contrast, polymerization kinetics of monomers with smaller cations, piperidinium (pipNB) and trimethylammonium (tmaNB), more closely resembled first order kinetics (FIG. 14 and FIG. 20), leading to near-complete conversion of monomer and ion incorporations of 35 mol % and 26 mol %, respectively (Table 2). This reduced reactivity of endo monomers with bulkier cations is consistent with a hypothesis that adding steric bulk to the endo isomer further restricts its ability to polymerize, though other factors may also be in effect. Nonetheless, all four of these direct insertion polymerizations resulted in polymers with significant ion incorporations and indicated IEC^{theo} values which matched those determined by ¹H NMR of each polymer (Table 2).

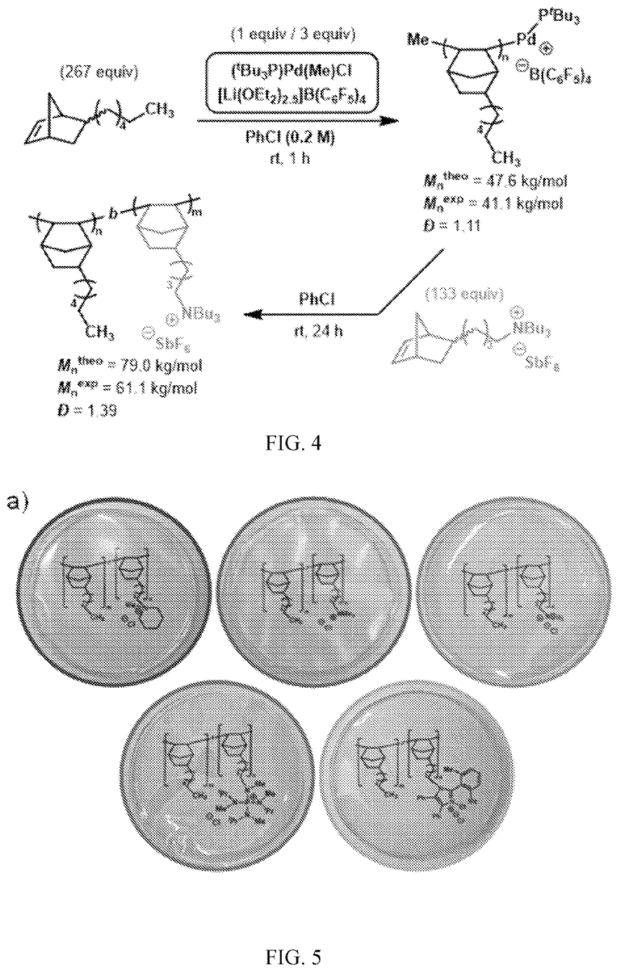

Multiblock copolymers have previously shown enhanced ion transport in AEMs, thus, investigation of the ability of this direct insertion polymerization to form block copolymer structures was pursued. Combining hexNB, catalyst, and activator in chlorobenzene for 1 hour resulted in >99% conversion of hexNB to give a polymer with a Ð of 1.11 and an Mₙ^{exp} of 41.1 kg/mol, which was in excellent agreement with the Mₙ^{theo} of 47.6 kg/mol (FIG. 4). Subsequent addition of tbaNB resulted in a slower polymerization, reaching 71% monomer conversion in 24 hours to give a block copolymer with a slightly broadened Ð of 1.39 and an Mₙ^{exp} of 61.1 kg/mol, which was in good agreement with the Mₙ^{theo} of 79.0 kg/mol (FIG. 24). This experiment not only demonstrates that block copolymer structures can be accessed, but it also shows that homopolymerization of ionic monomers can be achieved using this direct insertion polymerization strategy.

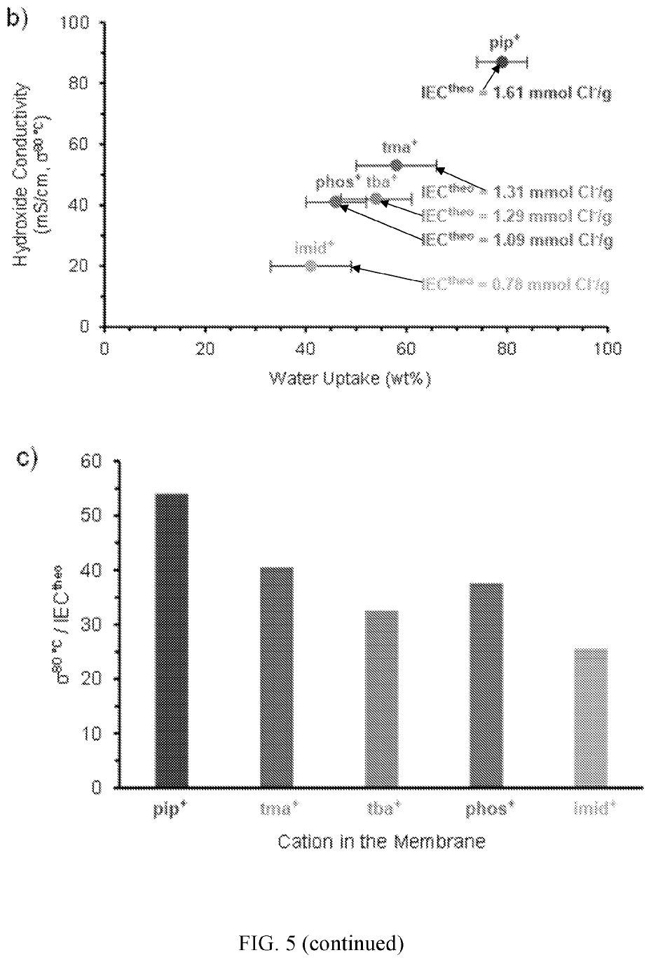

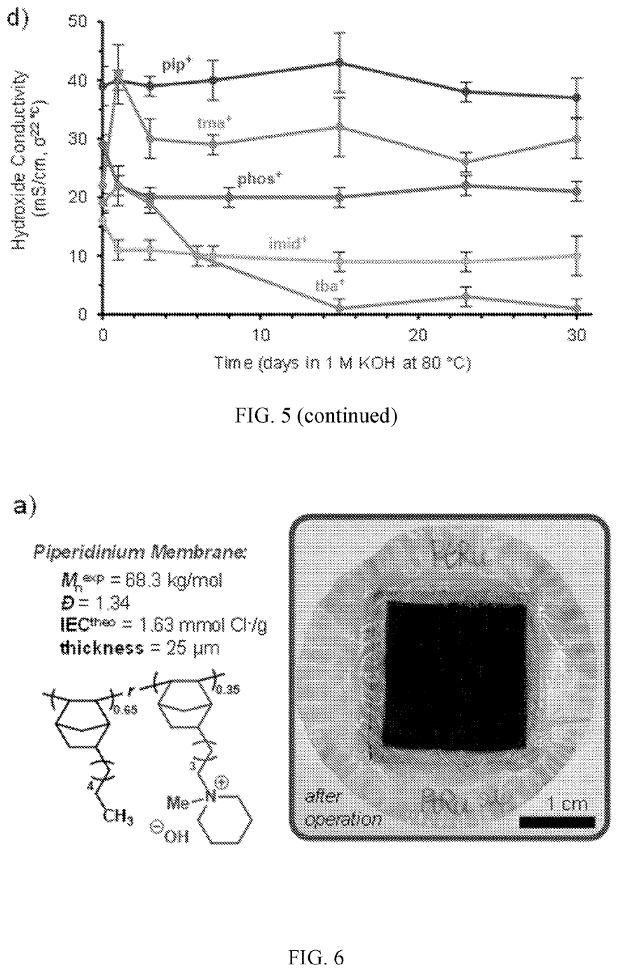

AEM Characterization. To gain insight into the effect of cation identity on hydroxide conductivity and stability, polymer samples were all solvent-cast into thin films and measured hydroxide ion conductivity using electrochemical impedance spectroscopy (FIG. 5a). Across the series of polymers, hydroxide ion conductivity at 80° C. (σ^{80° C.}) increased with IEC^{theo} and water uptake (WU), where piperidinium (pip⁺) resulted in the membrane with the highest σ^{80° C.} of 87 mS/cm, with an IEC^{theo} of 1.61 mmol Cl⁻/g and a WU of 79 wt % (FIG. 5b, Table 2). Importantly, when looking at the ratio of σ^{80° C.} to IEC^{theo}, the pip⁺ membrane also exhibited the highest value, showing an improvement over membranes containing other cations (FIG. 5c). This ratio was Across all polymers, the hydration number (λ) remained relatively constant, only varying between 23 and 29. Despite a wide range of WU values, the swelling ratio (ΔL) of each membrane was approximately 10, except for the imidazolium (imid⁺) membrane for which ΔL was not determined due to extreme brittleness of the dried polymer.

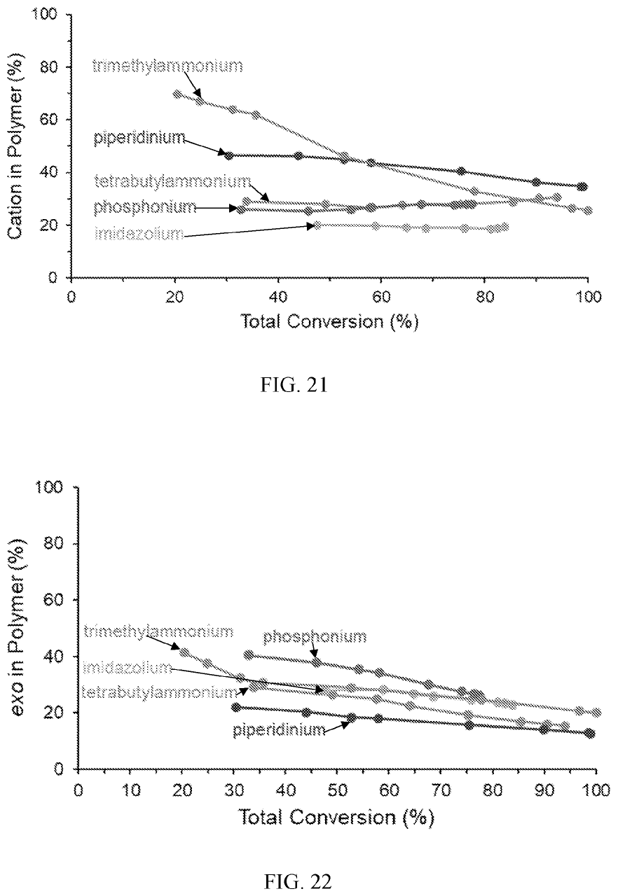

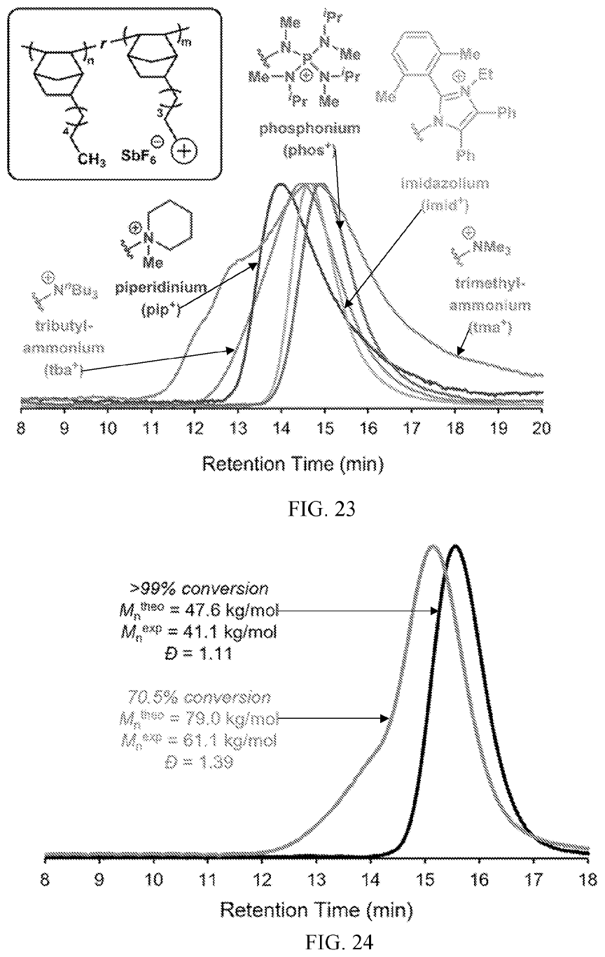

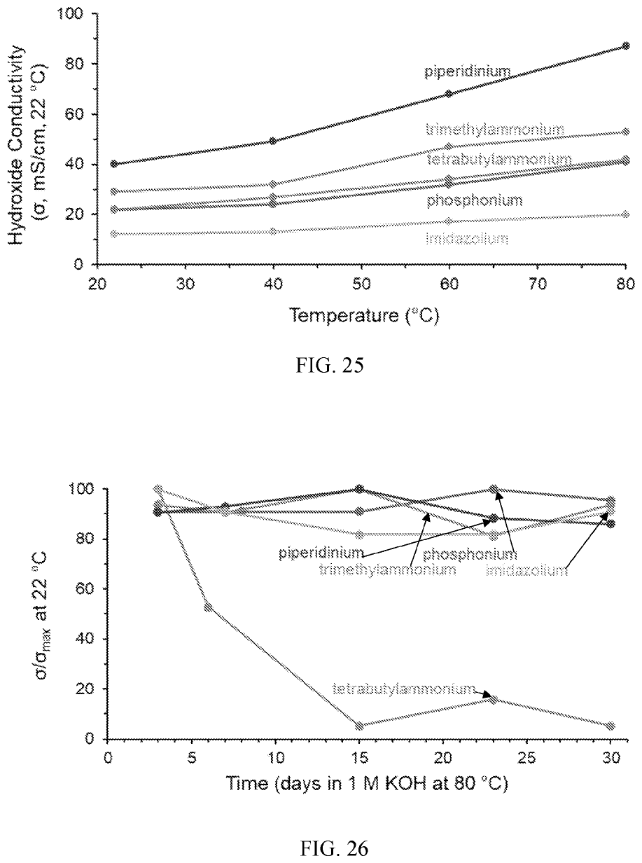

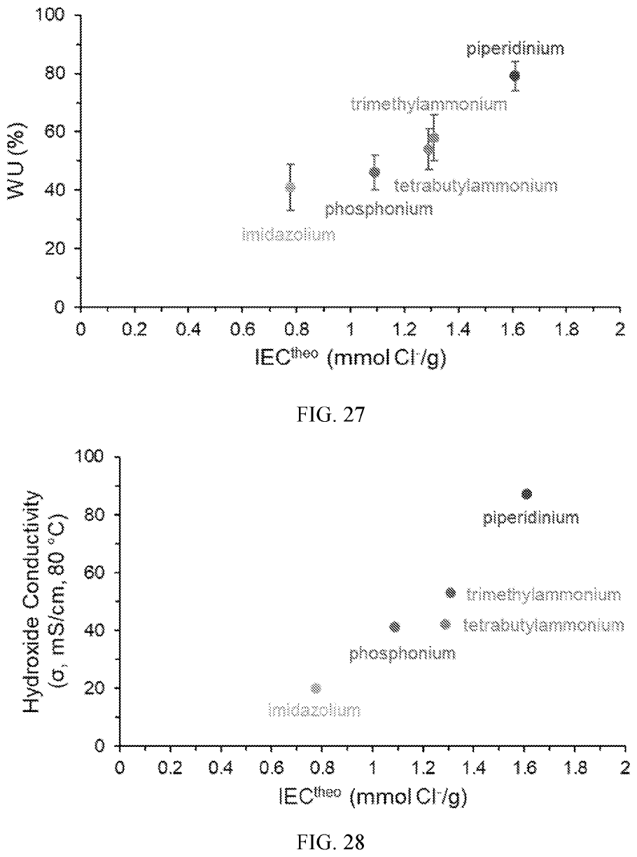

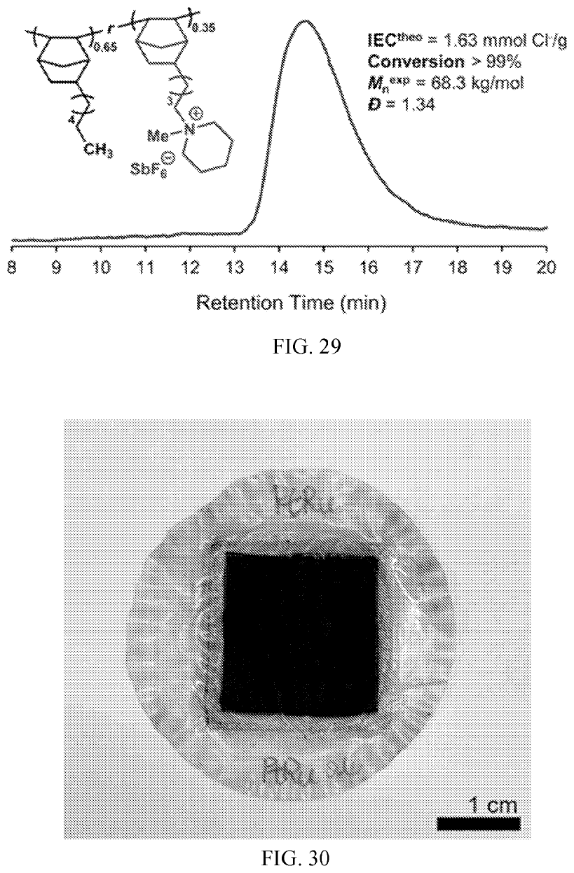

Noonan and co-workers have previously synthesized PNB AEMs with pendent phosphonium (phos⁺) and trimethylammonium (tma⁺) cations via post-polymerization functionalization; thus, the instant materials were compared with those materials. For a phos⁺ membrane, values of both the λ and ratio of σ^{80° C.} to IEC^{theo} were in good agreement with the values reported by Noonan and co-workers (29 and 46, respectively). For the tma⁺ membrane, however, the λ agreed more closely with the value reported for the post-polymerization functionalization material with a diblock copolymer structure (λ of 26) rather than that with a statistical copolymer structure (λ of 10). In addition, the instant ratio of σ^{80° C.} to IEC^{theo} was between those reported for the diblock and statistical copolymers (46 and 38, respectively). Indeed, it was observed faster conversion of tmaNB in its copolymerization with hexNB (FIG. 3f), and it was hypothesized this difference in reactivity resulted in a gradient or tapered structure (FIG. 21). Interestingly, kinetics of all other polymerizations indicated a consistent incorporation of neutral and cationic monomers across the polymer chain; thus, it was proposed that a gradient structure only occurred with tmaNB and may be related to the observed polymer precipitation during the reaction.

To investigate the alkaline stability of this series of membranes, a 30-day conductivity/stability study was conducted (FIG. 5d and FIG. 26). Membranes with pip⁺, tma⁺, phos⁺, and imid⁺ were stable in 1 M KOH at 80° C. with no significant loss in hydroxide conductivity over 30 days. However, the conductivity of the tributylammonium (tba⁺) membrane decreased significantly after day 3. This result is consistent with previous small molecule studies by Marino and Kreuer, in which tetrapropylammonium was shown to be significantly less stable than tma⁺ in 6 M KOH/H₂O. This difference in stability was attributed to the susceptibility of the propyl groups to undergo β-elimination, which was proposed is the primary degradation pathway for the instant tba⁺ membrane. Notably, these results are not consistent with previous small molecule studies by Coates and co-workers, in which tba⁺ was found to be more stable than tetradecyltrimethylammonium in 1 M KOH/CD₃OH. This discrepancy highlights the importance of hydroxide concentration and solvent effects in alkaline stability studies.

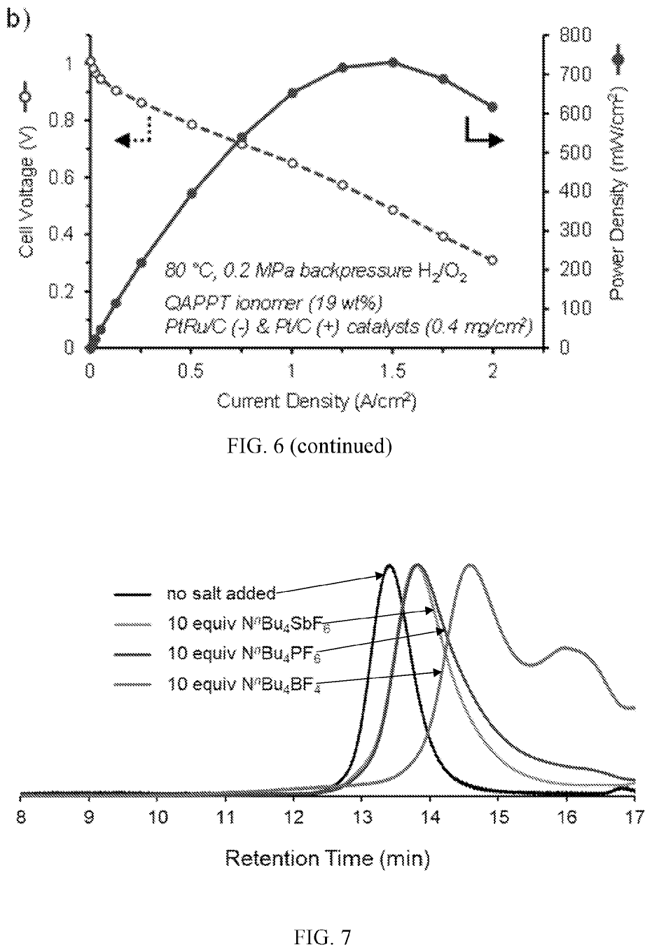

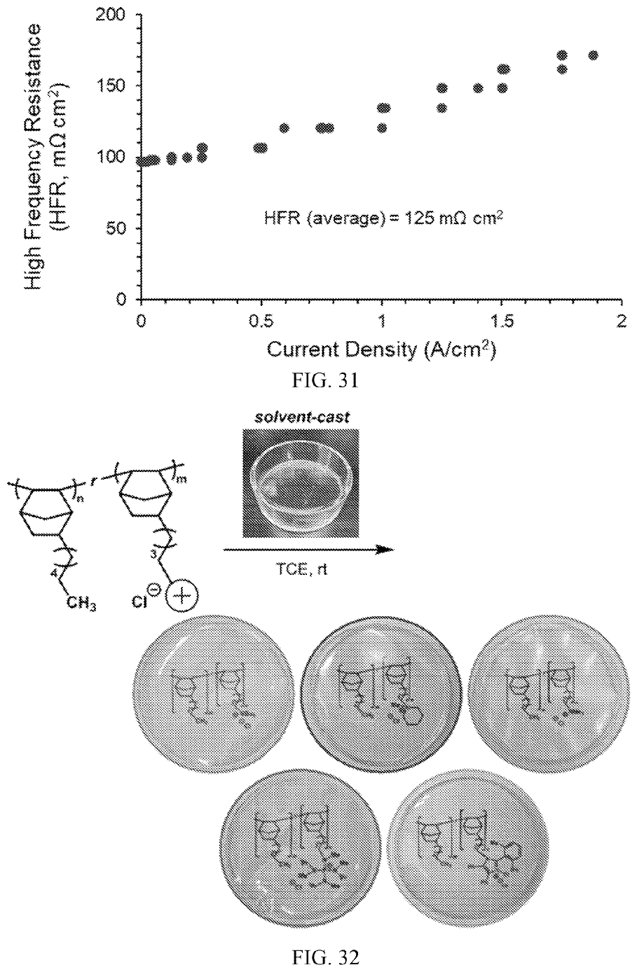

Finally, to demonstrate the performance of a membrane synthesized using this new method, the highest performing membrane was integrated into a working AEMFC. A second AEM with pip⁺ was synthesized as previously described, which resulted in a 68.3 kg/mol polymer with a Ð of 1.34 and IEC^{theo} of 1.63 mmol Cl⁻/g. The counterion was then exchanged to chloride and solvent-cast the polymer into a 25 μm-thick film before constructing the membrane electrode assembly (MEA) and exchanging the membrane to the hydroxide form (FIG. 6a). The performance of the MEA was tested at 80° C. with 100% relative humidity and 0.2 MPa backpressure of pure hydrogen and oxygen at the anode and cathode, respectively. A peak power density of 730 mW/cm² was observed (FIG. 6b). This unoptimized fuel cell performance can be compared to the current state-of-the-art, and to our knowledge represents the highest peak power density reported from a solution processable, aliphatic polymer-based AEM. In addition, a high-frequency resistance (HFR) of 125 mΩ cm² was observed (FIG. 31). In more recent work by Kohl and co-workers on their crosslinked PNB materials containing tma⁺, significant improvements in peak power density (0.510 W/cm² to 3.4 W/cm²) and HFR (95 mΩ cm² to 20 mΩ cm²) were achieved after optimization. Likewise, it was anticipated that fuel cell performance will increase significantly with further optimization of ionomer, electrodes, membrane thickness, feed gas relative humidity, and reacting gas dew point.

A direct coordination-insertion polymerization of ionic monomers that achieves high ion incorporation was developed. Importantly, this represents the first direct synthesis of aliphatic polymer-based AEMs, eliminating the need for post-polymerization modifications and allowing facile access to a broad range of materials. Kinetic studies revealed that more sterically demanding pendent cations slowed the homopolymerization rate of the endo isomer. Using this direct polymerization strategy, a diverse library of AEMs was synthesized and the influence of cation identity on hydroxide conductivity and stability was investigated. Membranes with pip⁺ exhibited high alkaline stability and resulted in the highest ratio of hydroxide conductivity to IEC^{theo}, with a hydroxide ion conductivity of 87 mS/cm² at 80° C. and IEC^{theo} of 1.61 mmol Cl⁻/g. Furthermore, integration of an instant pip⁺ membrane into an alkaline fuel cell device resulted in a peak power density of 730 mW/cm².

General Reagent Information. Palladium(II) chloride (99.9%, Strem), 1,5-cyclooctadiene (99%, Sigma-Aldrich), tetramethyltin (98%, Alfa Aesar), tri-tert-butylphosphine (96%, Alfa Aesar), 6-bromo-1-hexene (Oakwood), lithium tetrakis(pentafluorophenyl)borate ethyl etherate (Thermo Scientific), dicyclopentadiene (95%, Acros), 1-octene (98%, Sigma-Aldrich), tributylamine (99%, Fluka), trimethylamine (31-35% in ethanol, Fluka), potassium carbonate (99%, Fisher Scientific), tetrabutylammonium chloride (97%, Sigma-Aldrich), silver hexafluoroantimonate (99%, Alfa Aesar), Amberlite IRA-410 (chloride form, matrix styrene/divinylbenzene, 20-25 mesh, Sigma-Aldrich), benzil (99%, Thermo Scientific), ammonium acetate (97%, TCI), 2,6-dimethylbenzaldehyde (97%, TCI), L-proline (99%, Sigma-Aldrich), 1-bromoethane (99%, Sigma-Aldrich), calcium hydride (90%, BTC), chloroform (99%, Fisher Scientific), methanol (99%, Chem-Impex International), 1,1,2,2-tetrachloroethane (97%, TCI), iodomethane (Alfa Aesar), piperidine (≥99.5%, Sigma-Aldrich), aluminum oxide (activated, basic, Brockmann Grade I, 58 Å, Fisher Scientific), celite (454 Filter Aid, Fisher Scientific), and potassium hydroxide (Millipore) were used as received.

Tetrabutylammonium hexafluorophosphate (Bu₄NPF₆) (98%, TCI) and tetrabutylammonium tetrafluoroborate (Bu₄NBF₄) (98%, TCI) were purified by recrystallization from ethyl acetate three times and dried in vacuo at 60° C. for 18 h. Dichloromethane (DCM) (≥99.9%, B&J), acetonitrile (MeCN) (≥99.9%, B&J), and tetrahydrofuran (THF) (≥99.9%, Macron Fine Chemicals) were purchased from Avantor and were purified by first vigorously purging with argon for 2 h (h=hour(s)) and then passing through two packed columns of neutral alumina under argon pressure. Chlorobenzene (99%, Alfa Aesar) and anisole (Millipore) were dried over calcium hydride for 16 h, distilled into a Schlenk flask, freeze-pump-thawed three times, and stored in a nitrogen filled glovebox. Deuterated solvents for NMR were purchased from Cambridge Isotope Laboratories. All reactions and manipulations of air or water sensitive compounds were carried out under dry nitrogen from Airgas using a Braun UniLab drybox or standard Schlenk techniques unless otherwise specified.

General Analytical Information. Nuclear Magnetic Resonance (NMR) Spectroscopy. ¹H, ¹³C, ¹⁹F, and ³¹P NMR spectra were collected in deuterated solvents on a Varian INOVA 400 MHz or Bruker 500 MHz NMR spectrometer at 22° C. Chemical shifts were reported relative to the residual solvent peaks: chloroform-d (CDCl₃) 7.26 ppm (¹H) and 77.16 ppm (¹³C), acetone-d₆ ((CD₃)₂CO) 2.05 ppm (¹H) and 29.84 ppm (¹³C), or 1,1,2,2-tetrachloroethane-d₂ (TCE-d₂) 6.0 ppm (¹H) and 73.78 ppm (¹³C). Data for ¹H NMR are reported as follows: chemical shift (δ ppm), broad peak (br), apparent (appr.) multiplicity (s=singlet, d=doublet, t=triplet, q=quartet, m=multiplet), coupling constant (Hz) and integration; data for ¹³C NMR are reported in terms of chemical shift and no special nomenclature is used for equivalent carbons.

Mass Spectrometry (MS). High resolution mass spectrometry analyses were performed on a Thermo Scientific Exactive Orbitrap MS system equipped with an Ion Sense DART ion source (DART-HRMS) at 450° C. under helium gas. Monomers and counterions were analyzed using positive or negative ion mode, respectively.

To determine whether each ion exchange of halide to hexafluoroantimonate was quantitative, DART-HRMS was first performed on a sample of each monomer in halide-counterion form to ensure that any halide present would be detected. Bromide and iodide were detected; however, the mass of chloride was below the mass limit of the instrument, thus DART-HRMS could not be used to determine the efficiency of ion exchange for phosNB. DART-HRMS was then performed on each monomer after ion exchange, showing hexafluoroantimonate and no peak from the original halide, indicating that the counterion swap to hexafluoroantimonate was quantitative for tbaNB, imidNB, pipNB, and tmaNB.

Gel-Permeation Chromatography (GPC). GPC measurements of ionic polymers were performed on a Waters Instrument equipped with a 2690 autosampler, a Waters 2414 refractive index (RI) detector, and two SDV columns (Porosity 1000 and 100000 Å; Polymer Standard Services). The eluent (THF) was doped with 10 mM lithium bis(trifluoromethanesulfonyl)imide (flow rate of 1 mL/min, 40° C.). A 9-point calibration based on polystyrene standards (Polystyrene, ReadyCal Kit, Polymer Standard Services) was applied for determination of molecular weights. All GPC samples were prepared by dissolving ~5 mg polymer with SbF₆⁻ counterions into 2 mL of the GPC eluent before being Filtered through a 0.22 μm PTFE syringe filter.

All non-ionic polymers were analyzed using a Tosoh EcoSEC HLC 8320 GPC system with two SuperHM-M columns in series at a flow rate of 0.350 mL/min. Tetrahydrofuran was used as the eluent and number-average molecular weights (Mₙ), weight-average molecular weights (M_{w}), and dispersities (Ð) for all samples were determined by refractive index (RI) detection calibrated against a series of 12 TSKgel polystyrene standards.

TABLE 4
Conversion data and GPC values for all polymerizations of hexNB in the presence
of different tetrabutylammonium salts. Polymerizations with SbF₆⁻, PF₆⁻, and
BF₄⁻ were performed in triplicate with error calculated to 95% confidence.

| | Conversion | Std. | Mₙ^{theo} | Mₙ^{exp} | Std. | | Std. |
|---|---|---|---|---|---|---|---|
| Anion | (%) | Dev. | (kg/mol) | (kg/mol) | Dev. | Ð | Dev. |
| none | >99 | N.A. | 35.7 | 27.4 | N.A. | 1.18 | N.A. |
| SbF₆⁻ | 58 ± 6.0 | 3.6 | 20.7 | 14.9 ± 1.79 | 1.06 | 1.58 ± 0.0801 | 0.0475 |
| PF₆⁻ | 67 ± 11 | 6.7 | 23.8 | 11.7 ± 3.71 | 2.20 | 1.97 ± 0.408 | 0.242 |
| BF₄⁻ | 17 ± 2.7 | 1.6 | 5.88 | 3.47 ± 0.425 | 0.252 | 3.33 ± 0.496 | 0.294 |
| Cl⁻ | <5 | N.A. | 0 | n.d. | N.A. | n.d. | N.A. |

Electrochemical Impedance Spectroscopy (EIS). The in-plane hydroxide conductivity of the anion exchange membrane (AEM) sample was measured by four-probe electrochemical impedance spectroscopy (EIS) using a Pine Wavedriver workstation along with Aftermath software. The four-point probe was purchased from BekkTeck LLC (Loveland, CO), and a helpful schematic and description of a similar experimental setup has been reported. Strips of the membranes in chloride form (ca. 4 cm long×0.5 cm wide) were converted to the hydroxide form by immersing them in 20 mL of 1 M potassium hydroxide in a PTFE bottle for a minimum of 12 h. The strips were transferred to a HPDE bottle and were allowed to stir in 2×30 mL portions of 1 M KOH solution for 40 minutes. Residual potassium hydroxide was washed away by immersing the membrane strips in 3×60 mL portions of deionized water in a HDPE bottle for 10 minutes each. The AEM was then clamped into the four-point probe using a Proto 6104 torque screwdriver set to 1 inch ounce and completely immersed in deionized water at 22° C. during the measurement time. EIS was performed by imposing a small sinusoidal (AC signal) voltage, 10 mV, across the membrane sample at frequencies between 100 kHz and 100 Hz (scanning from high to low frequencies) and measuring the resultant current response. In a Nyquist plot of the data, the high frequency intercept on the real impedance axis was taken to be the resistance of the membrane. This was then used to calculate the hydroxide conductivity by employing the following formula: σ=L/Z′×A where L is the length between sense electrodes (0.425 cm), Z′ is the real impedance response at high frequency, and A is the membrane area available for hydroxide conduction (width×thickness). The dimensional measurements were performed using a digital micrometer (±0.001 mm) purchased from Marathon Watch Company Ltd. (Richmond Hill, ON). Hydroxide conductivities for the 30-day conductivity-stability study were measured for a minimum of three separate strips (per composition).

### Water Uptake (WU) and Swelling Ratio (ΔL) Measurements.

The water uptake and dimensional change were measured by the change between the fully hydrated and dried AEM strips (ca. 4 cm long×0.5 cm wide). Conversion to the hydroxide form was achieved by immersing the AEM strips in 1 M KOH at 80° C. for a minimum of 16 h followed by stirring in 2×30 mL portions of 1 M KOH for 40 minutes each. Residual KOH was washed away by immersing the membranes in 3×60 mL portions of deionized water for 10 minutes each. Immediately following hydroxide ion exchange, a sample was gently wiped using a Kimwipe. Its weight was measured on the balance on a piece a weighing paper, and the length of the strip was measured with a ruler. The thin film (in the hydroxide form) was dried under high vacuum at 70° C. for 6 h to reach complete dehydration.

Cation Incorporation (catNB_{incorp}) Determined by ¹H NMR of Each Polymerization.

$${catNB}_{incorp} = \frac{{catNB}_{feed} * {catNB}_{conv}}{( {catNB}_{feed} * {catNB}_{conv} ) + ( {hexNB}_{feed} * {hexNB}_{conv} )}$$

where
catNB_{feed}=ionic monomer in the monomer feed at time zero (%)
catNB_{conv}=conversion of ionic monomer after 25 h (%)
hexNB_{feed}=hexNB in the monomer feed at time zero (%)
hexNB_{conv}=conversion of hexNB after 25 h (%)

### Theoretical Ion Exchange Capacity (IEC^{theo}) Calculation.

$${IEC}^{theo} = \frac{1 mol cation}{{MW}_{catNB} + ( {MW}_{hexNB} * \frac{1 - {catNB}_{incorp}}{{catNB}_{incorp}} )} * \frac{1000 mmol}{1 mol}$$

Cation Incorporation Determined by ¹H NMR of Each Polymer (catNB_{incorp,NMR}).

$${catNB}_{incorp , NMR} = \frac{\frac{{catNB}_{int}}{{catNB}_{theo}}}{\frac{{catNB}_{int}}{{catNB}_{theo}} + \frac{{hexNB}_{int}}{{hexNB}_{theo}}}$$

where
catNBᵢₙₜ=integration of a set of protons in the ionic monomer
catNBₜₕₑₒ=number of protons belonging to catNBᵢₙₜ in one monomer unit
hexNBᵢₙₜ=integration of a set of protons in hexNB
hexNBₜₕₑₒ=number of protons belonging to hexNBᵢₙₜ in one monomer unit

### Ion Exchange Capacity (IEC) Determined by ¹H NMR of Each Polymer.

$$IEC = \frac{1 mol cation}{{MW}_{catNB} + ( {MW}_{hexNB} * \frac{1 - {catNB}_{incorp , NMR}}{{catNB}_{incorp}} )} * \frac{1000 mmol}{1 mol}$$

Cation in Polymer (catNB_{polymer}) Calculation.

$${catNB}_{polymer} = \frac{{catNB}_{int , 0} - {catNB}_{int , t}}{{total}_{int , 0} - {total}_{int , t}}$$

$$where$$

catNB_{int,t0}=integration of alkenyl protons in the ionic monomer at time zero
catNBₜₕₑₒ=integration of alkenyl protons in the ionic monomer at time, t
total_{int,t0}=integration of alkenyl protons from all monomers at time zero
total_{int,t}=integration of alkenyl protons from all monomers at time, t
   Exo in Polymer (exoNB_{polymer}) Calculation.

$${exoNB}_{polymer} = \frac{{exoNB}_{int , 0} - {exoNB}_{int , t}}{{total}_{int , 0} - {total}_{int , t}}$$

where
exoNB_{int,t0}=integration of alkenyl protons in the exo monomers at time zero
exoNBₜₕₑₒ=integration of alkenyl protons in the exo monomers at time, t
total_{int,t0}=integration of alkenyl protons from all monomers at time zero
total_{int,t}=integration of alkenyl protons from all monomers at time, t

### Water Uptake (WU) Calculation.

$$WU = ( \frac{{mass}_{wet} - {mass}_{dry}}{{mass}_{dry}} )$$

### Hydration Number (λ) Calculation.

$$λ = \frac{WU * 1000}{18 * {IEC}^{theo}}$$

### Swelling Ratio (ΔL) Calculation.

$$Δ L = ( \frac{{length}_{wet} - {length}_{dry}}{{length}_{dry}} )$$

### Synthetic Procedures

### Synthesis of Tetrabutylammonium Hexafluoroantimonate

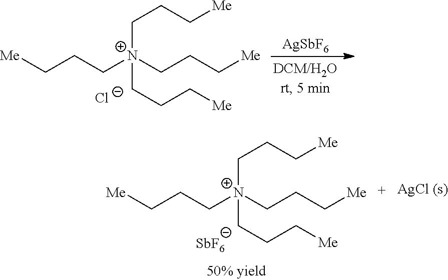

A 20 mL scintillation vial was charged with tetrabutylammonium chloride (0.10 g, 0.36 mmol, 1.0 equiv) followed by 10 mL of DCM and 5.0 mL of deionized water. Silver hexafluoroantimonate (0.16 g, 0.48 mmol, 1.3 equiv) was then added. The reaction mixture was shaken vigorously at room temperature for 5 minutes. The DCM layer was filtered by gravity filtration into a clean vial to remove all AgCl precipitate. 5.0 mL of deionized water was then added followed by excess silver hexafluoroantimonate (0.041 g, 0.12 mmol, 0.33 equiv). The mixture was shaken and filtered as done previously. The DCM layer was then exchanged for a third and final time with excess silver hexafluoroantimonate (0.041 g, 0.12 mmol, 0.33 equiv) before the DCM layer was filtered again and concentrated under reduced pressure. The product was recrystallized three times in ethyl acetate to give a white crystalline product (0.86 g, 0.18 mmol, 50% yield). ¹H NMR (500 MHz, (CD₃)₂CO): δ 3.43 (m, 2H), 1.82 (m, 2H), 1.44 (m, 2H), 0.98 (t, J=7.4 Hz, 3H). ¹⁹F NMR (470 MHz, (CD₃)₂CO): δ −123.23 (m, 6F).

### General Procedure for “Cracking” Dicyclopentadiene

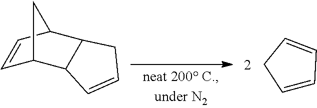 [SMILES: C1=CC2C3C=CC(C3)C2C1 ; C1=CCC=C1]

A 25 mL round bottom flask fit with a short path distillation apparatus was charged with ~10 mL of dicyclopentadiene and heated to 200° C. under flow of nitrogen. Cyclopentadiene was collected in a flask cooled to 0° C., and was used immediately.

### Synthesis of 5-hexylbicyclo[2.2.1]hept-2-ene

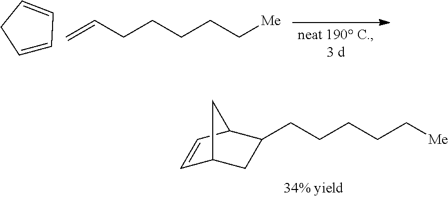

5-Hexylbicyclo[2.2.1]hept-2-ene was prepared according to a previous report. A 15 mL pressure flask was charged with cyclopentadiene (1.5 mL, 18 mmol, 1.0 equiv) followed by 1-octene (5.7 mL, 37 mmol, 2.0 equiv). The reaction was sealed, stirred, and heated to 190° C. for 3 d (d=day(s)) in an oil bath. Upon completion, the reaction was allowed to cool and was purified by fractional distillation under reduced pressure to return excess 1-octene and the pure product, each as a clear liquid (1.1 g, 6.2 mmol, 34% yield). 5-hexylbicyclo[2.2.1]hept-2-ene was then dried over calcium hydride for 16 h, distilled into a Schlenk bomb, freeze-pump-thawed three times, and stored in a nitrogen filled glovebox. The ¹H NMR spectrum matched that which was previously reported, where the ratio of endo/exo isomers was similar (80:20 endo:exo). ¹H NMR (500 MHz, CDCl₃): δ 6.10 (dd, J=5.7, 3.0 Hz, 0.8H), 6.08 (dd, J=5.7, 3.0 Hz, 0.2H), 6.01 (dd, J=5.7, 3.0 Hz, 0.2H), 5.91 (dd, J=5.7, 3.0 Hz, 0.8H), 2.75 (m, 1.8H), 2.50 (s, 0.2H), 1.96 (m, 0.8H), 1.82 (m, 0.8H), 1.37 (m, 1H), 1.25 (m, 9.8H), 1.05 (m, 1.8H), 0.88 (m. 3H), 0.48 (m, 0.8H).

### Synthesis of 5-(4-bromobutyl)bicyclo[2.2.1]hept-2-ene

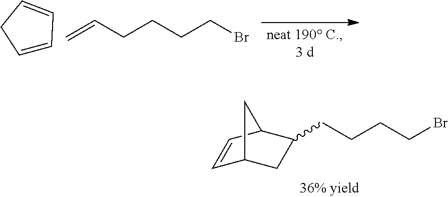

5-(4-Bromobutyl)bicyclo[2.2.1]hept-2-ene was prepared according to a previous report. A 15 mL pressure flask was charged with cyclopentadiene (1.5 mL, 18 mmol, 1.0 equiv) followed by 6-bromo-1-hexene (6.9 mL, 37 mmol, 2.0 equiv). The reaction was sealed, stirred, and heated to 190° C. for 3 d in an oil bath. Upon completion, the reaction was allowed to cool and was purified by fractional distillation under reduced pressure to give the desired product. The product was further purified using column chromatography on silica gel with ethyl acetate/hexanes (20:80) as the eluent (no pressure was applied to the column) to give a clear, viscous liquid (1.5 g, 6.6 mmol, 36% yield). The ¹H NMR spectrum matched that which was previously reported, where the ratio of endo/exo isomers was similar (75:25 endo:exo). ¹H NMR (500 MHz, CDCl₃): δ 6.11 (dd, J=5.7, 3.0 Hz, 0.8H), 6.08 (dd, J=5.7, 3.1 Hz. 0.2H), 6.02 (dd, J=5.7, 2.9 Hz, 0.2H), 5.91 (dd, J=5.7, 2.9 Hz, 0.8H), 3.40 (m. 2H), 2.76 (m, 1.75H), 2.51 (s, 0.25H), 1.97 (m, 1H), 1.84 (m, 3H), 1.43 (m, 3H), 1.21 (d, J=8.0 Hz, 1H), 1.10 (m, 2H), 0.48 (m, 1H).

### Synthesis of 4-(bicyclo[2.2.1]hept-5-en-2-yl)-N,N,N-tributylbutan-1-aminium Hexafluoroantimonate

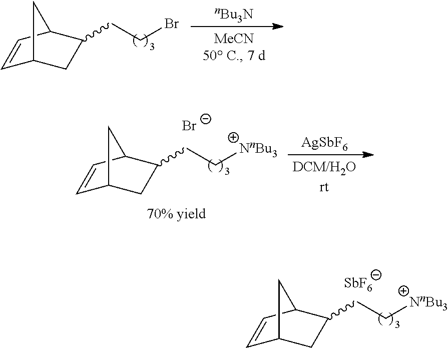

A 20 mL scintillation vial with a Teflon-lined cap was charged with 5-(4-bromobutyl)bicyclo[2.2.1]hept-2-ene (1.0 g, 4.4 mmol, 1.0 equiv) followed by tributylamine (1.2 mL, 5.2 mmol, 1.2 equiv) and acetonitrile (5.0 mL). The reaction mixture was stirred and heated to 50° C. for 7 d in an oil bath. It should be noted that retro-Diels-Alder products were observed at higher temperatures. The reaction was allowed to cool before all unreacted starting materials were extracted in three washes of hexanes with a small amount of added potassium carbonate (~50 mg), leaving only the desired product dissolved in acetonitrile. The acetonitrile layer was then evaporated under reduced pressure to yield the product as an orange-red oil. (1.3 g, 3.1 mmol, 70% yield). This monomer in bromide form was then exchanged to hexafluoroantimonate form following the ion exchange procedure detailed below. HRMS (DART-MS): Calculated for C₂₃H₄₄N⁺ [M]⁺ 334.34683, Found 334.346079. ¹H NMR (500 MHz, (CD₃)₂CO): δ 6.11 (dd, J=5.8, 3.1 Hz, 0.8H), 6.06 (dd, J=5.7, 3.0 Hz, 0.2H), 6.01 (dd, J=5.7, 3.0 Hz, 0.2H), 5.91 (dd, J=5.8, 2.9 Hz, 0.8H), 3.44 (m, 8H), 2.75 (m, 2H), 2.50 (s, 0.2H), 1.83 (m, 8H), 1.42 (m, 9H), 1.22 (m, 5H), 0.98 (t, J=7.4 Hz, 9H), 0.48 (m, 0.8H). ¹⁹F NMR (470 MHz, (CD₃)₂CO): δ −122.87 (m, 6F). ¹³C NMR (126 MHz, (CD₃)₂CO): δ 137.81, 137.44, 136.97, 132.87, 59.61, 59.34, 50.13, 47.02, 46.06, 45.75, 43.25, 42.58, 39.40, 39.29, 36.52, 34.91, 33.50, 32.90, 26.08, 24.40, 22.63, 20.38, 13.83.

### Synthesis of 1-(4-(bicyclo[2.2.1]hept-5-en-2-yl)butyl)piperidine

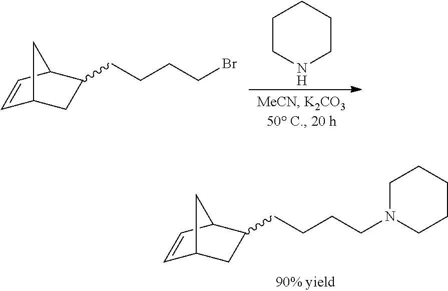 [SMILES: BrCCCCC1CC2C=CC1C2 ; C1CCNCC1 ; C1=CC2CC1CC2CCCCN1CCCCC1]

A 20 mL scintillation vial was charged with 5-(4-bromobutyl)bicyclo[2.2.1]hept-2-ene (1.0 g, 4.4 mmol, 1.0 equiv) followed by acetonitrile (5.0 mL). To this solution, K₂CO₃ (1.0 g, 7.2 mmol, 1.6 equiv) was added followed by piperidine (1.1 mL, 11 mmol, 2.5 equiv). A blanket of nitrogen was applied over the solution and the vial was capped with a Teflon-lined cap and wrapped with electrical tape. The reaction mixture was allowed to stir at 50° C. for 20 h. Upon completion, the reaction was allowed to cool and was concentrated under reduced pressure. Hexanes (~5 mL) were added to the vial and the mixture was filtered through celite, then through basic alumina. The solution was concentrated and dried to give the desired product as a clear oil (0.92 g, 3.9 mmol, 90% yield). HRMS (DART-MS): Calculated for C₁₆H₂₈N⁺ [M+H]⁺ 234.22217, Found 234.220267. ¹H NMR (500 MHz, CDCl₃): δ 6.09 (dd, J=5.8, 3.1 Hz, 0.8H), 6.06 (dd, J=5.7, 3.1 Hz, 0.2H), 6.00 (dd, J=5.8, 2.9 Hz, 0.2H), 5.89 (dd, J=5.8, 2.9 Hz, 0.8H), 2.74 (m, 2H), 2.49 (s, 0.2H), 2.34 (s, 3H), 2.24 (t, J=7.9 Hz, 2H), 1.96 (m, 1H), 1.81 (m, 1H), 1.57 (m, 4H), 1.35 (m, 9H), 1.06 (m, 2H), 0.46 (m, 0.8H). ¹³C NMR (126 MHz, CDCl₃): δ 137.02, 136.30, 132.52, 59.85, 54.80, 49.67, 46.43, 45.49, 45.33, 42.64, 41.99, 38.81, 38.79, 36.67, 34.86, 33.20, 32.53, 27.35, 27.33, 27.16, 26.93, 26.14, 24.66.

### Synthesis of 1-(4-(bicyclo[2.2.1]hept-5-en-2-yl)butyl)-1-methylpiperidin-1-ium Hexafluoroantimonate

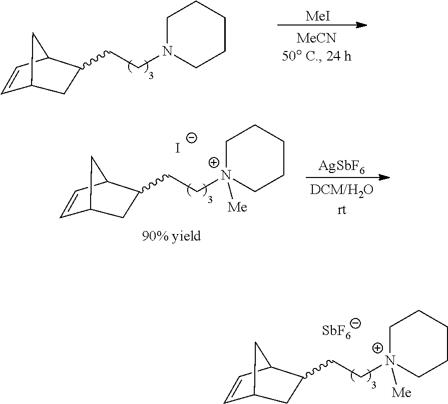

A 20 mL scintillation vial was charged with 1-(4-(bicyclo[2.2.1]hept-5-en-2-yl)butyl)piperidine (0.92 g, 3.9 mmol, 1.0 equiv) followed by acetonitrile (5.0 mL). To this solution, iodomethane (0.49 mL, 7.9 mmol, 2.0 equiv) was added. A blanket of nitrogen was applied over the solution and the vial was capped with a Teflon-lined cap and wrapped with electrical tape. The reaction mixture was allowed to stir at 50° C. for 24 h. Upon completion, the reaction was allowed to cool and was concentrated under reduced pressure. The resulting yellow powder was filtered and washed with ether (~10 mL) three times to afford the desired product as a white powder (1.3 g, 3.5 mmol, 90% yield). This monomer in iodide form was then exchanged to hexafluoroantimonate form following the ion exchange procedure detailed below. HRMS (DART-MS): Calculated for C₁₇H₃₀N⁺ [M]⁺ 248.23782, Found 248.236647. ¹H NMR (500 MHz, (CD₃)₂CO): δ 6.11 (dd, J=6.0, 3.1 Hz, 0.8H), 6.06 (dd, J=5.9, 3.1 Hz, 0.2H), 6.00 (dd, J=5.9, 3.0 Hz, 0.2H), 5.92 (dd, J=5.9, 3.0 Hz, 0.8H), 3.53 (m, 6H), 3.23 (s, 3H), 2.76 (m, 2H), 2.50 (s, 0.2H), 1.99 (m, 4H), 1.85 (m, 3H), 1.72 (m, 2H), 1.33 (m, 7H), 0.49 (m, 0.8H). ¹⁹F NMR (470 MHz, (CD₃)₂CO): δ −123.05 (m, 6F). ¹³C NMR (126 MHz, (CD₃)₂CO): δ 137.71, 137.49, 136.89, 132.92, 64.55, 61.93, 61.91, 61.89, 61.87, 50.09, 48.38, 47.03, 46.00, 45.72, 43.22, 42.56, 39.35, 39.19, 36.61, 34.93, 33.51, 32.91, 26.30, 26.07, 22.57, 22.56, 21.76, 20.59.

### Synthesis of ((4-(bicyclo[2.2.1]hept-5-en-2-yl)butyl)(methyl)amino)tris(isopropyl(methyl)amino)phosphonium Hexafluoroantimonate

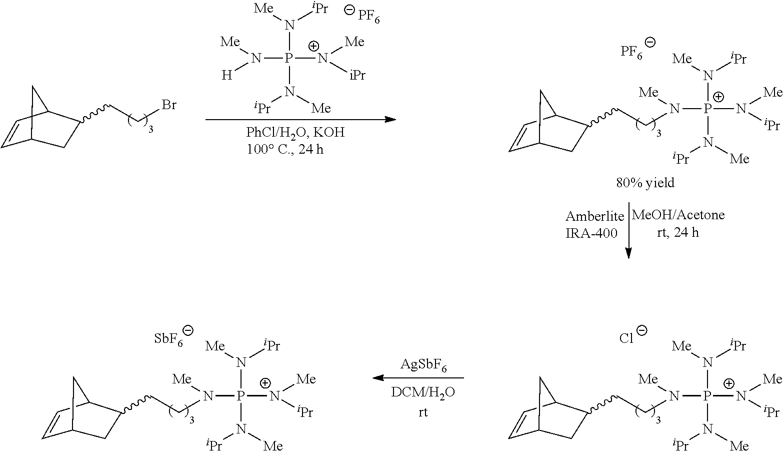

Tris(isopropyl(methyl)amino)(methylamino)phosphonium hexafluorophosphate(V) was prepared according to a previous report. Tris(isopropyl(methyl)amino)(methylamino)phosphonium hexafluorophosphate(V) (2.6 g, 6.2 mmol, 1.0 equiv), chlorobenzene (13 mL), 50% (w/w) KOH/H₂O solution (13 g total), and 5-(4-bromobutyl)norbornene (3.5 g, 15 mmol, 2.5 equiv) were added to a 50 mL flask. The flask was placed in an oil bath set to 100° C. and the reaction mixture was stirred for 24 h. An aliquot (~0.5 mL) was removed from the organic layer and analyzed by ³¹P NMR spectroscopy, revealing complete conversion to the desired product. The flask was allowed to cool, and the mixture was transferred to a separatory funnel where it was diluted with water (50 mL) and dichloromethane (50 mL). The organic layer was separated, and the aqueous layer was extracted with dichloromethane (50 mL) twice more. The organic layers were then combined and washed three times with a saturated KPF6 solution (50 mL) and once with water (50 mL). The combined organic layers were dried over anhydrous sodium sulfate and then concentrated under reduced pressure. The resulting oil was precipitated into diethyl ether (200 mL) and the desired product was collected by vacuum filtration as an off-white powder (2.8 g, 4.9 mmol, 80% yield).

((4-(Bicyclo[2.2.1]hept-5-en-2-yl)butyl)(methyl)amino)tris(isopropyl(methyl)amino)phosphon-ium hexafluorophosphate was dissolved in a 50% v/v methanol/acetone solution. A 5:1 mass ratio of Amberlite IRA-400 resin was added, and the mixture was stirred for 24 h. Exchange to chloride was monitored by ³¹P NMR, and more resin was added as needed. The resin was then filtered off and the solvent was evaporated to produce the monomer in chloride form. This monomer in chloride form was then exchanged to hexafluoroantimonate form following the ion exchange procedure detailed below. HRMS (DART-MS): Calculated for C₂₄H₅₀N₄P⁺ [M]⁺ 425.37676, Found 425.375699. ¹H NMR (500 MHz, (CD₃)₂CO): δ 6.11 (dd, J=5.8, 3.1 Hz, 0.8H), 6.06 (dd, J=6.0, 3.1 Hz, 0.2H), 6.00 (dd, J=5.9, 3.0 Hz, 0.2H), 5.91 (dd, J=6.0, 3.1 Hz, 0.8H), 3.69 (m, 3H), 3.07 (m, 2H), 2.86 (m, 3H), 2.75 (d, J=9.8 Hz, 9H), 2.50 (s, 0.2H), 2.02 (m, 1H), 1.85 (m, 1H), 1.69 (m, 2H), 1.28 (m, 26H), 0.48 (m, 0.8H). ¹⁹F NMR (470 MHz, (CD₃)₂CO): δ −123.33 (m, 6F). ³¹P NMR (202 MHz, (CD₃)₂CO): δ 44.28 (s, 1P). ¹³C NMR (126 MHz, (CD₃)₂CO): δ 137.68, 137.50, 136.89, 132.93, 50.16, 50.11, 47.79, 47.74, 47.04, 46.07, 45.73, 43.24, 42.57, 39.48, 39.34, 36.91, 35.72, 35.69, 35.25, 33.58, 32.97, 28.94, 28.91, 28.72, 28.70, 26.87, 26.61, 20.09, 20.07.

### Synthesis of 2-(2,6-dimethylphenyl)-4,5-diphenyl-1H-imidazole

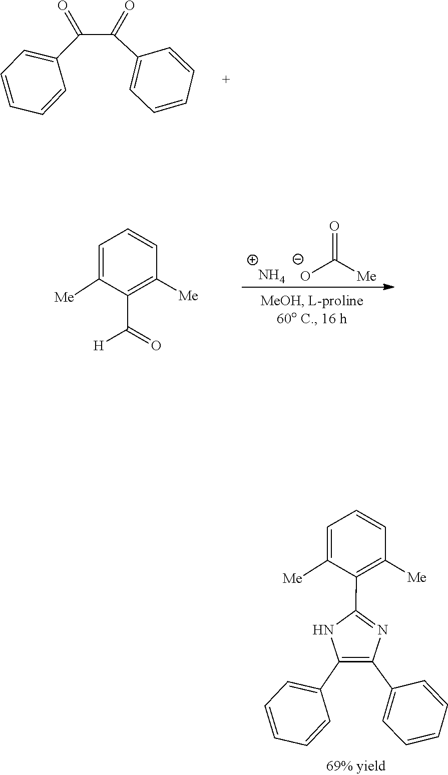

2-(2,6-Dimethylphenyl)-4,5-diphenyl-1H-imidazole was prepared using the following modified procedure from a previous report. Benzil (3.5 g, 17 mmol, 1.0 equiv), 2,6-dimethylbenzaldehyde (2.2 g, 17 mmol, 1.0 equiv), ammonium acetate (6.4 g, 83 mmol, 5.0 equiv), L-proline (0.29 g, 2.5 mmol, 0.15 equiv) and methanol (24 mL) were added to a 125 mL flask. The flask was stirred and heated to 60° C. for 16 h in an oil bath. During the reaction, formation of the product as a white precipitate was observed. The precipitate was isolated from the crude reaction mixture by vacuum filtration and then recrystallized from methanol/water. The pure product was isolated as white crystals, which were dried in vacuo at 60° C. for 4 h (3.7 g, 11 mmol, 69% yield). HRMS (DART-MS): Calculated C₂₃H₂₁N₂⁺ [M+H]⁺ 325.17047, Found 325.172341. ¹H NMR (500 MHz, TCE-d₂): δ 7.59 (br, 4H), 7.37 (m, 4H), 7.31 (m, 3H), 7.17 (d, J=7.62 Hz, 2H), 2.24 (s, 6H). ¹³C NMR (126 MHz, TCE-d₂): δ 144.73, 138.61, 130.56, 129.35, 128.51, 127.54, 127.47, 127.25, 20.23.

### Synthesis of 1-(4-(-bicyclo[2.2.1]hept-5-en-2-yl)butyl)-2-(2,6-dimethylphenyl)-4,5-diphenyl-1H-imidazole

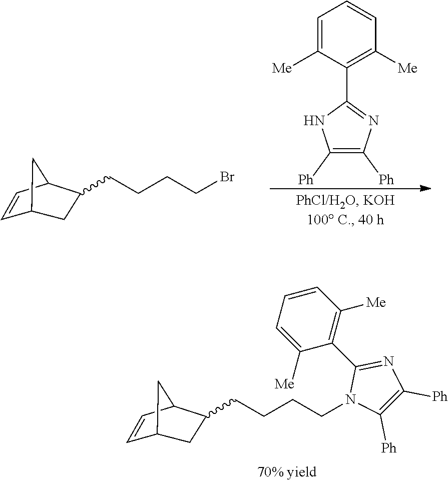 [SMILES: BrCCCCC1CC2C=CC1C2 ; Cc1cccc(C)c1-c1nc(-c2ccccc2)c(-c2ccccc2)[nH]1 ; Cc1cccc(C)c1-c1nc(-c2ccccc2)c(-c2ccccc2)n1CCCCC1CC2C=CC1C2]

2-(2,6-Dimethylphenyl)-4,5-diphenyl-1H-imidazole (2.4 g, 7.3 mmol, 1.0 equiv), chlorobenzene (29 mL), 50% (w/w) KOH/H₂O solution (13 g total), and 5-(4-bromobutyl)norbornene (2.0 g, 8.9 mmol, 1.2 equiv) were added to a 100 mL flask equipped with a reflux condenser. The flask was placed in an oil bath set to 100° C. and the reaction mixture was stirred for 40 h. The flask was then allowed to cool, and the mixture was transferred to a separatory funnel where it was diluted with water (50 mL) and dichloromethane (50 mL). The organic layer was separated, and the aqueous layer was extracted with dichloromethane (50 mL) twice more. The organic layers were combined and washed with water (150 mL) three times. The combined organic layers were dried over anhydrous sodium sulfate and then concentrated under reduced pressure. Residual chlorobenzene was removed in vacuo at 60° C. for 2 h to produce a dark brown viscous oil. The oil was further purified using column chromatography on silica gel with a hexanes/ethyl acetate gradient as the eluent to separate the desired product from residual starting materials. The solvent was removed under reduced pressure to give the product as a yellow oil, which was further dried in vacuo at 60° C. for 6 h to produce a white solid (2.4 g, 5.2 mmol, 70% yield). HRMS (DART-MS): Calculated for C₃₄H₃₇N₂⁺ [M+H]⁺ 473.29567, Found 473.292625. ¹H NMR (500 MHz, CDCl₃): δ 7.55 (m, 2H), 7.48 (m, 3H), 7.43 (m, 2H), 7.25 (m, 1H), 7.19 (m, 2H), 7.12 (m, 3H), 6.02 (dd, J=5.6, 3.1 Hz, 0.85H), 5.99 (dd, J=5.7, 3.0 Hz, 0.15H), 5.96 (dd, J=5.7, 2.8 Hz, 0.15H), 5.73 (dd, J=5.7, 3.0 Hz, 0.85H), 3.49 (m, 2H), 2.70 (s, 0.15H), 2.67 (s, 0.85H), 2.51 (s, 0.85H), 2.28 (s, 0.15H), 2.22 (s, 6H), 1.64 (m, 2H), 1.30 (m, 1H), 1.21 (m, 2H), 1.11 (m, 1H), 0.87 (m, 2H), 0.69 (m, 2H), 0.28 (m, 1H). ¹³C NMR (126 MHz, CDCl₃): δ 146.09, 138.83, 137.33, 137.09, 136.82, 136.31, 135.07, 132.23, 132.01, 131.21, 131.02, 129.30, 129.15, 128.65, 128.63, 128.32, 128.14, 127.64, 126.77, 126.14, 49.57, 46.19, 45.28, 45.21, 44.20, 42.54, 41.88, 38.37, 38.18, 35.49, 33.66, 32.95, 32.26, 30.36, 30.28, 25.65, 25.40, 20.28.

### Synthesis of 1-(4-(bicyclo[2.2.1]hept-5-en-2-yl)butyl)-2-(2,6-dimethylphenyl)-3-ethyl-4,5-diphenyl-1H-imidazol-3-ium Hexafluoroantimonate

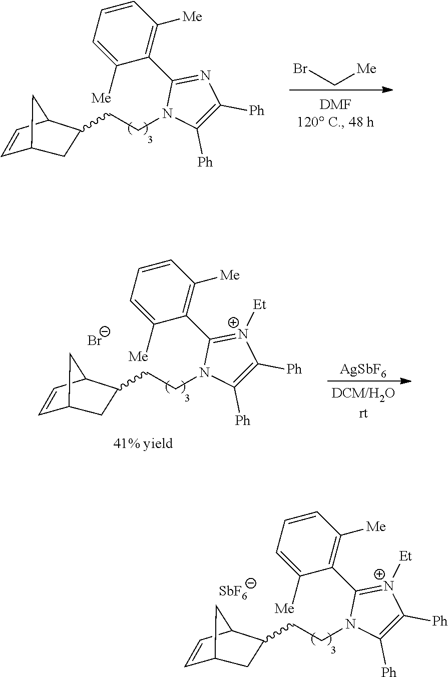

1-(4-(Bicyclo[2.2.1]hept-5-en-2-yl)butyl)-2-(2,6-dimethylphenyl)-4,5-diphenyl-1H-imidazole (1.8 g, 3.8 mmol, 1.0 equiv), 1-bromoethane (0.46 g, 4.2 mmol, 1.1 equiv), and dimethylformamide (8 mL) were added to a 125 mL pressure vessel equipped with a stir bar. The pressure vessel was sealed, stirred, and heated to 120° C. in an oil bath for 48 h. The reaction mixture was then added dropwise to diethyl ether (150 mL) to give a light brown precipitate. The precipitate was further purified using column chromatography on silica gel with an ethyl acetate/acetone gradient as the eluent to separate the desired product from residual starting materials. The solvent was removed under reduced pressure to give the product as a yellow oil, which was further dried in vacuo at 60° C. for 6 h to produce a light-yellow solid (0.91 g, 1.6 mmol, 41% yield). This monomer in bromide form was then exchanged to hexafluoroantimonate form following the ion exchange procedure detailed below. HRMS (DART-MS): Calculated for C₃₆H₄₁N₂⁺ [M]⁺ 501.32643, Found 501.325250. ¹H NMR (500 MHz, (CD₃)₂CO): δ 7.66 (m, 5H), 7.52 (m, 6H), 7.48 (m, 2H), 6.01 (dd, J=5.7, 3.0 Hz, 0.85H), 5.98 (dd, J=5.6, 3.1 Hz, 0.15H), 5.95 (dd, J=5.7, 3.0 Hz, 0.15H), 5.73 (dd, J=5.7, 3.0 Hz, 0.85H), 4.11 (q. J=7.2 Hz, 2H), 4.02 (m, 2H), 2.67 (s, 0.15H), 2.64 (s, 0.85H), 2.51 (s, 0.85), 2.37 (s, 6H), 2.27 (s, 0.15H), 1.66 (m, 2H), 1.45 (m, 2H), 1.27 (m, 1H), 1.15 (s, 0.15H), 1.10 (m, 1H), 1.06 (t, J=7.2 Hz, 3H), 1.01 (t, J=7.3 Hz, 2H), 0.87 (m, 0.30H), 0.72 (m, 1.70H), 0.26 (m, 0.85H). ¹⁹F NMR (470 MHz, (CD₃)₂CO): δ −122.92 (m, 6F). ¹³C NMR (126 MHz, (CD₃)₂CO): δ 144.19, 140.26, 137.61, 137.37, 136.78, 133.85, 133.69, 133.42, 132.72, 132.02, 131.95, 131.27, 131.25, 129.94, 129.85, 126.68, 126.59, 122.20, 49.96, 47.37, 46.78, 45.80, 45.58, 43.10, 43.04, 42.45, 38.84, 38.58, 35.68, 33.93, 33.32, 32.70, 29.77, 25.83, 25.54, 20.09, 14.93.

### Synthesis of 4-(bicyclo[2.2.1]hept-5-en-2-yl)-N,N,N-trimethylbutan-1-aminium Hexafluoroantimonate

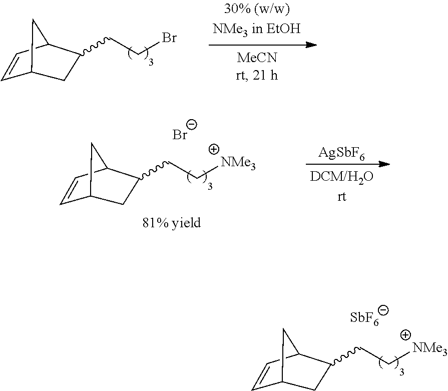

A 20 mL scintillation vial with a Teflon-lined cap was charged with 5-(4-bromobutyl)bicyclo[2.2.1]hept-2-ene (0.75 g, 3.3 mmol, 1.0 equiv) followed by 30% (w/w) trimethylamine/ethanol (1.2 g, 5.9 mmol, 1.8 equiv) and acetonitrile (5.0 mL). The reaction was stirred at room temperature for 21 h. The crude reaction mixture was transferred to a separatory funnel where it was diluted with water (20 mL) and chloroform (20 mL). The organic layer was separated, and the aqueous layer was extracted with chloroform (20 mL) twice more. All organic phases were combined and concentrated under reduced pressure to give the desired product as a white powder. (0.76 g, 2.7 mmol, 810% yield). This monomer in bromide form was then exchanged to hexafluoroantimonate form following the ion exchange procedure detailed below. HRMS (DART-MS): Calculated for C₁₄H₂₆N⁺ [M]⁺ 208.20598, Found 208.205650. ¹H NMR (500 MHz, (CD₃)₂CO): δ 6.10 (dd, J=5.9, 3.1 Hz, 0.8H), 6.07 (dd, J=6.0, 3.1 Hz, 0.2H), 6.00 (dd, J=5.9, 3.0 Hz, 0.2H), 5.95 (dd, J=5.8, 3.1 Hz, 0.8H), 3.73 (m, 2H), 3.44 (m, 9H), 2.87 (s, 0.2H), 2.78 (s, 0.8H), 2.76 (s, 0.2H), 2.72 (s, 0.8H), 2.52 (s, 0.2H), 1.86 (m, 3H), 1.33 (m, 7H), 0.49 (m, 0.8H). ¹⁹F NMR (470 MHz, (CD₃)₂CO): δ −123.17 (m, 6F). ¹³C NMR (126 MHz, (CD₃)₂CO): δ 137.62, 137.53, 136.83, 133.03, 66.90, 53.22, 50.07, 47.03, 45.99, 45.75, 43.21, 42.56, 39.37, 39.16, 36.64, 34.96, 33.54, 32.94, 26.20, 25.95, 23.69.

### General Procedure for the Quantitative Ion Exchange of Halides to Hexafluoroantimonate

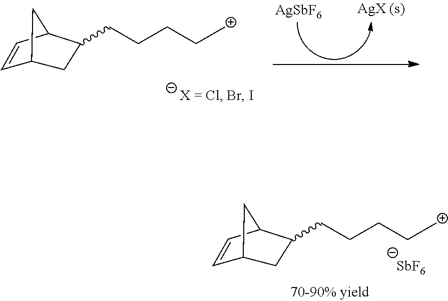

To a separatory funnel, dichloromethane was added along with the ionic monomer of choice (1.0 equiv). Deionized water was then added to the separatory funnel followed by AgSbF₆ (1.1 equiv). The separatory funnel was vigorously shaken. The organic layer was then drained through a filter and the remaining aqueous layer was washed three times with smaller amounts of dichloromethane, which were then filtered and combined with the initial organic fraction. The precipitate (AgX) mostly remained in the aqueous layer. After discarding this aqueous fraction, the collected organic fraction was added back into the separatory funnel along with fresh water and additional AgSbF₆ (0.2 equiv). Again, the separatory funnel was shaken vigorously, and the organic layer was filtered and collected. A third and final wash was performed with fresh water and AgSbF₆ (0.2 equiv) such that the monomer was washed with a total of 1.5 equiv of AgSbF₆. The organic fraction was concentrated under reduced pressure, then dried on a Schlenk line overnight to give the final product (~70-90% yield). DART-MS was performed according to the procedure in the analytical information section to confirm that the exchange was quantitative. If the exchange was incomplete, the above procedure was repeated until no halide was detected by DART-MS.

### Synthesis of chloro(tri-tert-butylphosphine)methylpalladium(II)

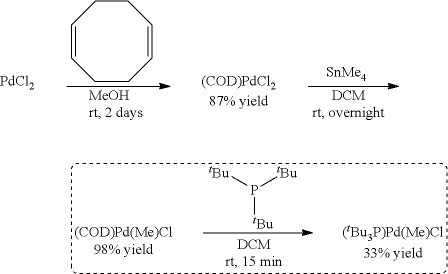

Chloro(1,5-cyclooctadiene)methylpalladium(II) was prepared according to a previous report. Chloro(tri-tert-butylphosphine)methylpalladium(II) was prepared according to the following modified procedure from a previous report. In a nitrogen filled glovebox, a 1-dram vial was charged with chloro(1,5-cyclooctadiene)methylpalladium(II) (0.053 g, 0.20 mmol, 1.0 equiv) followed by tri-tert-butyl phosphine (0.040 g, 0.20 mmol, 1.0 equiv) and dichloromethane (1.0 mL). The reaction mixture was stirred for 15 minutes at room temperature. The solution was then pumped out of the glovebox and transferred to a reaction tube. The solution was partially concentrated under reduced pressure before hexanes were carefully layered on top of the remaining dichloromethane for liquid diffusion recrystallization at −20° C. The resulting orange crystals were isolated, dried, and stored in a sealed vessel at −20° C. under nitrogen and in the dark (24 mg, 0.066 mmol, 33% yield). The ¹H NMR and ³¹P NMR spectra matched those which were previously reported. ¹H NMR (500 MHz, CDCl₃): δ 1.77 (d, J=0.8 Hz, 3H), 1.51 (d, J=12.7 Hz, 27H). 31P NMR (202 MHz, CDCl₃): δ 69.67 (s, 1P). ¹³C NMR (126 MHz, (CD₃)₂CO): δ 39.95, 31.97, 1.72.

### Polymerization Procedures

General Procedure for the Polymerization of 5-hexylbicyclo[2.2.1]hept-2-ene in the Presence of Different Tetrabutylammonium Salts

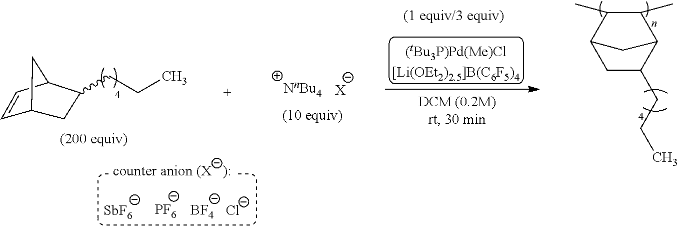

Chloro(tri-tert-butylphosphine)methylpalladium(II) (1.0 mg, 0.0028 mmol, 12 equiv) and lithium tetrakis(pentafluorophenyl)borate ethyl etherate (7.3 mg, 0.0084 mmol, 36 equiv) were added together into a 1-dram vial, which was capped with a septum and dried on a Schlenk line overnight at room temperature. Each tetrabutylammonium salt (0.0023 mmol, 10 equiv) was separately weighed into a 1-dram vial, which was capped with a septum and also dried on a Schlenk line overnight at room temperature. After drying, all vials were pumped into a nitrogen filled glovebox. Reactions with hexafluoroantimonate, hexafluorophosphate, and tetrafluoroborate were run in triplicate.

A stock solution containing 5-hexylbicyclo[2.2.1]hept-2-ene (0.50 mL, 0.44 g, 2.5 mmol, 11*10³ equiv), anisole (0.064 mL, 0.59 mmol, 2.5*10³ equiv), and dichloromethane (0.436 mL) was prepared and analyzed by NMR to gain the monomer to anisole (internal standard) ratio at time zero. To each 1-dram vial containing a tetrabutylammonium salt, a portion of the above stock solution (0.019 mL, 200 equiv monomer) was added along with a stir bar and additional dichloromethane (0.013 mL), such that the final concentration would be 0.2 M in monomer.

To the 1-dram vial containing palladium catalyst, dichloromethane (2.4 mL) was added, and the vial was allowed to stir for 30 minutes. This catalyst solution (0.20 mL, 1.0 equiv catalyst/3.0 equiv activator) was then added to each monomer solution to initiate polymerization. After 30 min, each polymerization was terminated by addition of acetonitrile (~5 drops) and was analyzed by NMR and THF GPC.

### General Procedure for the Direct Insertion Polymerization of Ionic Monomers

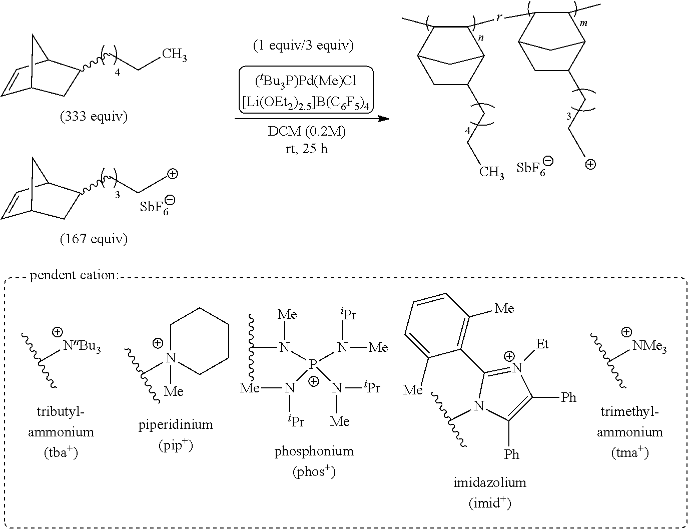

Chloro(tri-tert-butylphosphine)methylpalladium(II) (5.5 mg, 0.015 mmol, 5.5 equiv) and lithium tetrakis(pentafluorophenyl)borate ethyl etherate (40 mg, 0.046 mmol, 17 equiv) were added together into a 1-dram vial, which was capped with a septum and dried on a Schlenk line overnight at room temperature. Each ionic monomer (0.465 mmol, 167 equiv) was separately weighed into a 20 mL scintillation vial, which was capped with a septum and also dried on a Schlenk line overnight at room temperature. All vials were then pumped into a nitrogen filled glovebox.

To each 20 mL scintillation vial containing an ionic monomer, 5-hexylbicyclo[2.2.1]hept-2-ene (0.189 mL, 0.927 mmol, 333 equiv), anisole (0.038 mL, 0.035 mmol, 130 equiv), and dichloromethane (6.232 mL) were added, such that the final concentration would be 0.2 M in monomer. A stir bar was added, and an aliquot was taken for NMR analysis to gain the monomer to anisole (internal standard) ratio at time zero.

To the 1-dram vial containing palladium catalyst, dichloromethane (2.75 mL) was added, and the vial was allowed to stir for 30 minutes. This catalyst solution (0.50 mL, 1.0 equiv catalyst/3.0 equiv activator) was then added to each monomer solution to initiate polymerization. Additional aliquots were taken for NMR analysis over the course of 25 h before the polymerization was terminated by addition of acetonitrile (~20 drops) and was analyzed by THF GPC. The resulting polymer sample was precipitated into cold methanol, centrifuged, and isolated (×2) before being dried under reduced pressure.

### Procedure for the Formation of a Diblock Copolymer

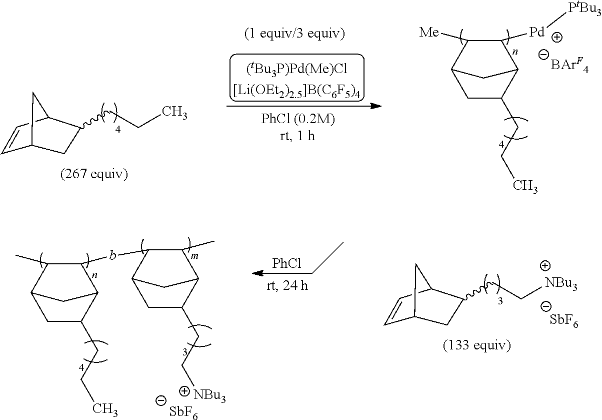

Chloro(tri-tert-butylphosphine)methylpalladium(II) (1.2 mg, 0.0033 mmol, 1.2 equiv) and lithium tetrakis(pentafluorophenyl)borate ethyl etherate (8.7 mg, 0.010 mmol, 3.6 equiv) were added together into a 1-dram vial, which was capped with a septum and dried on a Schlenk line overnight at room temperature. 4-(bicyclo[2.2.1]hept-5-en-2-yl)-N,N,N-tributylbutan-1-aminium hexafluoroantimonate (212 mg, 0.371 mmol, 133 equiv) was separately weighed into a 1-dram vial, which was capped with a septum and also dried on a Schlenk line overnight at room temperature. Both vials were then pumped into a nitrogen filled glovebox.

To a 20 mL scintillation vial, 5-hexylbicyclo[2.2.1]hept-2-ene (0.151 mL, 0.74 mmol, 267 equiv), anisole (0.030 mL, 0.028 mmol, 100 equiv), and chlorobenzene (3.035 mL) were added, such that the concentration was 0.2 M in monomer. A stir bar was added and an aliquot was taken for NMR analysis to gain the monomer to anisole (internal standard) ratio at time zero.

To the 1-dram vial containing palladium catalyst, chlorobenzene (0.60 mL) was added, and the vial was allowed to stir for 30 minutes. This catalyst solution (0.50 mL) was then added to the monomer solution to initiate polymerization. After 1 h, an aliquot was taken for NMR and THF GPC analysis. 4-(Bicyclo[2.2.1]hept-5-en-2-yl)-N,N,N-tributylbutan-1-aminium hexafluoroantimonate (211.7 mg, 0.371 mmol, 133 equiv) was then added for chain extension of the first block. Immediately after this addition, another aliquot was taken for NMR analysis to determine the monomer to anisole (internal standard) ratio. After 24 h, the polymerization was terminated by addition of acetonitrile (~20 drops) and was analyzed by NMR and THF GPC. The resulting polymer sample was precipitated into cold methanol, centrifuged, and isolated (×2) before being dried under reduced pressure.

Polymer Film Preparation. General Procedure for the Ion Exchange of Hexafluoroantimonate to Chloride

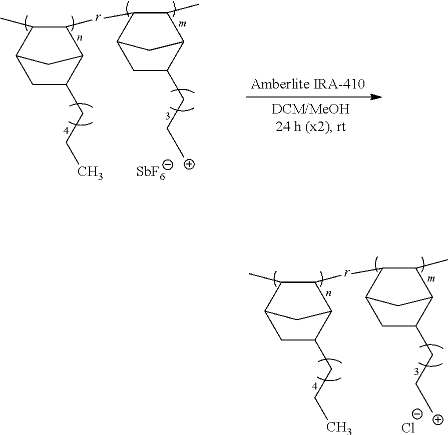

A 20 mL scintillation vial was charged with ionic polymer in hexafluoroantimonate form (100-300 mg) and dichloromethane (10 mL). After the polymer was completely dissolved (with the exception of the trimethylammonium membrane, which was only partially solubilized), a chloride exchange resin, Amberlite IRA-410 (approximately twice the mass of the polymer), was added along with methanol (10 mL). If the polymer started to precipitate upon addition of methanol, methanol addition was halted. This mixture was then allowed to stir at room temperature for 24 h. In the case of trimethylammonium, chloroform was used in place of dichloromethane, and the solution remained cloudy at first but turned clear after 24 h. The solution was then gravity filtered and evaporated under reduced pressure to return the exchanged polymer. The above procedure was repeated a second time to ensure complete exchange.

General Procedure for solvent casting polymer films, Each polymer in chloride form (100-200 mg) was dissolved in a minimum amount of tetrachloroethane before being passed through a syringe filter (0.45 μm) into a custom-made glass dish with a flat bottom (4.7 cm inner diameter). A fine wire mesh was placed on the top of each casting dish to exclude dust particles. Solvent was allowed to evaporate for several days at room temperature, and the resulting polymer film was removed from the casting dish with the addition of a small amount of water. In the case of piperidinium and trimethylammonium, solvent casting was performed using a mixture of tetrachloroethane and chloroform, with a small amount of methanol due to poor solubility in tetrachloroethane alone. In the case of imidazolium, casting was performed in tetrachloroethane with a small amount of methanol, and the film was removed/handled after only 1 day due to extreme brittleness if left to completely dry.

Membrane Electrode Assembly (MEA) Fabrication and Fuel Cell Test. The membrane electrode assembly was fabricated using QAPPT (quaternary ammonia poly(N-methyl-piperidine-co-p-terphenyl)) as ionomer, and PtRu/C (60%) and Pt/C (60%) catalysts (from fuel cell store) in the anode and cathode, respectively. The catalysts were mixed with QAPPT ionomer and dispersed in isopropanol in an ultrasonic bath, where the ionomer weight content was controlled to be 20%. These catalyst inks were sprayed onto the piperidinium membrane surface using a homemade ultrasonic spray system, forming an MEA. The ink was delivered to the ultrasonic nozzle (Siansonic Z402) with a flow rate of 0.05 mL/min, the ultrasonic power was kept at 3 W. The temperature of the hot plate was kept at 60° C. The precious metal loading in both the anode and the cathode was controlled to be 0.4 mg/cm² (including Pt and Ru).

The MEA was then exchanged to hydroxide form in a 1 M KOH solution at 70° C. in an oven under air for 12 h. The MEA was then sandwiched between AvCarb 3250 (from the fuel cell store) gas diffusion layer and PTFE gaskets (200 μm), and 4N·m torque was applied to the fuel cell fixture. The fuel cell test was then done with a Scribner 850e fuel cell test system. Pure hydrogen and oxygen were purged at the anode and the cathode with 100% relative humidity and a flow rate of 500 sccm. The current was gradually applied when the cell temperature reached 80° C. After the activation process, the cell potential was recorded at each current density.

While the peak power density obtained under the instant conditions (730 mW/cm²) was less than that found in recent reports of polynorbornene-based anion exchange membranes by Kohl and co-workers (3.4 W/cm²), it is similar to the peak power density of these same materials (0.510 mW/cm²) before significant optimization. The differences between the instant peak power density and the above best reported value can be attributed to a combination of the following factors: (1) The ion exchange capacity (IEC) of the instant membrane was much lower—1.63 meq/g compared to 3.37 meq/g. (2) The instant membrane was thicker—25 μm compared to a <20 μm. (3) The catalyst loadings used by Kohl and co-workers; 0.7 mg/cm² (anode) and 0.6 mg/cm² (cathode) were much higher than the 0.4 mg/cm² employed here. In addition, they are unrealistically high for practical applications. (4) Finally, QAPPT was used as the ionomer in the catalyst layer instead of a polynorbornene ionomer, which may have led to an unfavorable ionomer-membrane interface. Thus, as discussed above, performance increase is expected as these conditions are further optimized.

### Example 2

This Example describes, inter alia, cationic compounds (which may be monomers), cationic polymers and uses thereof, and methods of making cationic polymers.

Direct insertion polymerization of ion-containing norbornene monomers was demonstrated using the following catalyst system:

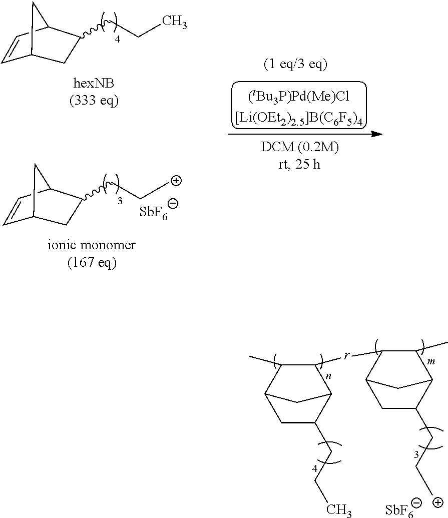

Direct insertion polymerization using a palladium catalyst with a different ligand, triadamantylphosphine, and using a different cocatalyst, AgSbF₆, was also demonstrated suggesting generality of palladium catalysts.

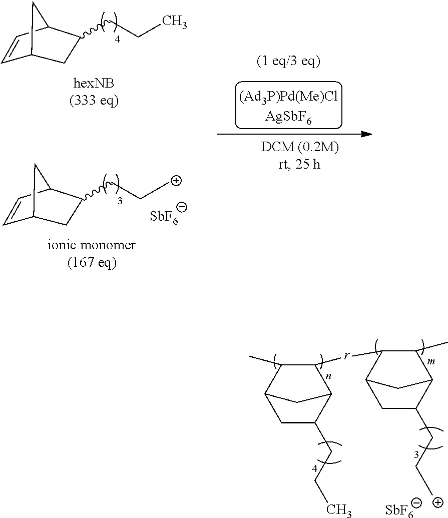

This direct insertion polymerization strategy is also expected to be applicable in polymerization of norbornenes with other metals, such as, for example, titanium, zirconium, iron, copper, nickel, chromium, cobalt, and the like, and any combination thereof.

Direct insertion polymerization of ion-containing norbornene monomers using BF₄, PF₆, SbF₆ was demonstrated. Weakly coordinating anions (such as, for example, the following and the like, and any combination thereof) are also expected to be able to be used.

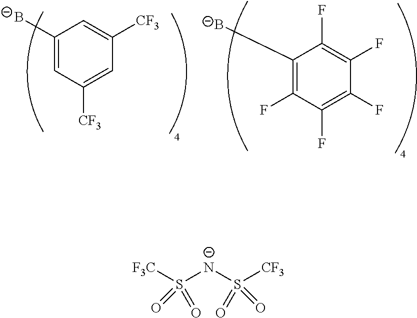

Direct insertion copolymerization of ion-containing norbornene monomers and the following neutral comonomer (using methods described in EXAMPLE 1) was also demonstrated.

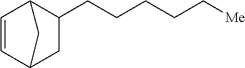 [SMILES: CCCCCCC1CC2C=CC1C2]

Direct insertion copolymerization of phosphonium-containing norbornene with an unsubstituted norbornene comonomer was demonstrated as shown below (using methods described in EXAMPLE 1). This allowed us to achieve full monomer conversion, which was not possible with the previous neutral comonomer.

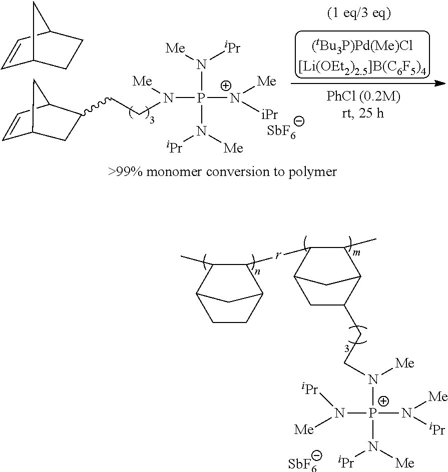

Production of the following polymers containing cyclic ammonium groups was demonstrated. These polymers were produced using methods described in EXAMPLE 1.

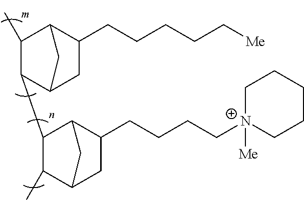

Although the present disclosure has been described with respect to one or more particular example(s), it will be understood that other examples of the present disclosure may be made without departing from the scope of the present disclosure.

1. A cationic compound comprising one or more norbornenyl group(s) and at least one cationic group, wherein each cationic group is independently at each occurrence chosen from phosphonium groups, imidazolium groups, cyclic ammonium groups, and wherein each cationic group is directly covalently linked or linked via a linking group to a norbornene ring of a norbornenyl group.

2. The cationic compound of claim 1, wherein each cationic group is directly covalently linked or linked via a linking group to the norbornene ring via a carbon other than a carbon of a carbon-carbon double bond of the norbornenyl group and/or each cationic group is directly covalently linked or linked via a linking group to the norbornene ring via a carbon at an endo or exo position other than a carbon of the carbon-carbon double bond of the norbornenyl group.

3. The cationic compound of claim 1, wherein each phosphonium group independently comprises a following structure: 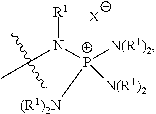
or a structural analog thereof, wherein
R¹ is independently at each occurrence chosen from H group, alkyl groups, cyclic alkyl groups, and, aryl groups; and
X⁻ is an anion or a complex anion.

4. The cationic compound of claim 1, wherein each imidazolium group independently comprises a following structure: 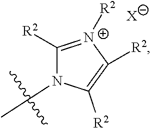
or a structural analog thereof, wherein
R² is independently at each occurrence chosen from H group, alkyl groups, cyclic alkyl groups, and aryl groups; and
X⁻ is an anion or a complex anion.

5. The cationic compound of claim 1, wherein each cyclic ammonium group independently comprises a following structure: 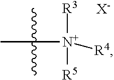
or a structural analog thereof, wherein
(i) R³, R⁴, and an ammonium nitrogen (N⁺) taken together form a heterocyclic group, wherein N⁺ is a member of a heterocyclic ring of the heterocyclic group and R⁵ is an aliphatic or aryl group, or (ii) R³, R⁴, R⁵, and N taken together form an aliphatic group-bridged heterocyclic group, wherein N⁺ is a member of a heterocyclic ring of the aliphatic group-bridged heterocyclic group; and
X⁻ is an anion or a complex anion.

6. The cationic compound of claim 1, wherein at least a portion or all of the cationic group(s) comprise a complex anion independently chosen from BF₄⁻, SbF₆⁻, SbCl₆⁻, PF₆⁻, B(Ar)₄⁻, wherein Ar is an aryl group, (Ar^{F})₄⁻, wherein Ar^{F} is an aryl group substituted with one, two, three, four, or five fluorine group(s) or one, two, three, four, or five fluoroalkyl group(s), and NTf₂⁻, wherein Tf is a triflate group.

7. The cationic compound of claim 1, wherein the cationic compound comprises 2, 3, 4, or 5 cationic groups covalently linked to a norbornenyl group.

8. The cationic compound of claim 1, wherein the cationic compound comprises at least two cationic groups covalently linked directly to each other via a linking group.

9. The cationic compound of claim 1, wherein the cationic compound comprises at least two cationic groups, wherein a first cationic group is covalently linked to a norbornenyl group and one or more second cationic groups(s) covalently linked to the first cationic group, wherein at least one of the cationic groups is a phosphonium group or an imidazolium group.

10. The cationic compound of claim 1, wherein the cationic compound comprises two cationic groups linked directly to each other via an aliphatic group, a heterocyclic group, wherein at least one N atom is quaternary, or a bridged heterocyclic group, wherein at least one N atom is quaternary atom of a first quaternary ammonium group and an aliphatic group, a heterocyclic group, wherein at least one N atom is a quaternary atom of second quaternary ammonium group, or a bridged heterocyclic group, where in at least one N atom is quaternary atom of a second quaternary group.

11. The cationic compound of claim 1, wherein the cationic compound comprises 2, 3, 4, or 5 norbornenyl groups covalently linked to a cationic group.

12. The cationic compound of claim 1, wherein the cationic compound comprises a cationic group and at least two norbornenyl groups,
wherein each of the norbornenyl groups are directly covalently linked or linked via a linking group to the cationic group.

13. The cationic compound of claim 1, wherein the cationic group comprises a cyclic cationic group fused to a norbornenyl group.

14. The cationic compound of claim 1, wherein the cationic compound comprises a following structure: 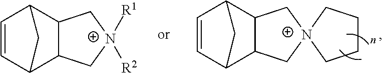
wherein n is 1, 2, 3, or 4, or a structural analog thereof.

15. The cationic compound of claim 1, wherein the cationic compound comprises a following structure: 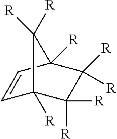
or a structural analog thereof, or a salt, a partial salt, a solvate, or a polymorph thereof, or a stereoisomer or a mixture of stereoisomers, an isotopic variant, or a tautomer thereof, wherein
R is independently at each occurrence chosen from H group, alkyl group, cationic group chosen from phosphonium groups, imidazolium groups, and cyclic ammonium groups,
with a proviso that at least one R group is a cationic group.

16. The cationic compound of claim 1, wherein the cationic compound comprises a following structure: 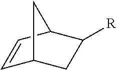
or a structural analog thereof, or a structural analog thereof, or a salt, a partial salt, a solvate, or a polymorph thereof, or a stereoisomer or a mixture of stereoisomers, an isotopic variant, or a tautomer thereof.

17. The cationic compound of claim 16, wherein an R group of the structure is in an endo or exo position.

18. (canceled)

19. (canceled)

20. (canceled)

21.-47. (canceled)
